(19) United States
(12) Patent Application Publication
(10) Pub. No.: US 2023/0381317 A1
(43) Pub. Date: Nov. 30, 2023

(54) METHODS FOR CONTROLLED ACTIVATION AND/OR EXPANSION OF GENETICALLY ENGINEERED CELLS USING POLYETHYLENE GLYCOL (PEG) RECEPTORS

(71) Applicant: Century Therapeutics, Inc., Philadelphia, PA (US)

(72) Inventors: Hyam Levitsky, Philadelphia, PA (US); Andreia Costa, Philadelphia, PA (US); John Wheeler, Philadelphia, PA (US); Melissa Chiasson, Philadelphia, PA (US); Eric Eymard, Philadelphia, PA (US); Rupesh Amin, Philadelphia, PA (US)

(73) Assignee: Century Therapeutics, Inc., Philadelphia, PA (US)

(21) Appl. No.: 18/303,672

(22) Filed: Apr. 20, 2023

Related U.S. Application Data
(60) Provisional application No. 63/333,165, filed on Apr. 21, 2022.

(51) Int. Cl. A61K 39/00; C12N 5/074; C12N 15/86; C12N 9/50; C07K 16/44; C07K 14/705; C07K 14/725

(52) U.S. Cl. CPC A61K 39/4646; C12N 5/0696; C12N 15/86; C12N 9/506; A61K 39/461; A61K 39/4631; A61K 39/4637; A61K 39/4634; A61K 39/464412; A61K 39/464413; A61K 39/464411; A61K 39/4635; C07K 16/44; C07K 14/70517; C07K 14/7051; C12N 2740/15043; C12N 2770/32022; C07K 2317/622; C07K 2317/31; A61K 2239/22; A61K 2239/28; A61K 2239/13; C07K 2319/03; C07K 2319/33

(57) ABSTRACT

Provided are genetically engineered induced pluripotent stem cells (iPSCs) and derivative cells thereof expressing a polyethylene glycol (PEG) receptors and methods of using the same. Also provided are compositions, polypeptides, vectors, and methods of manufacturing.

### CROSS REFERENCE TO RELATED APPLICATION

This application claims the benefit of U.S. Provisional Patent Application No. 63/333,165 filed Apr. 21, 2022, which is incorporated by reference herein in its entirety.

### TECHNICAL FIELD

This application provides methods for controlling the activation and expansion of genetically engineered induced pluripotent stem cells (iPSCs) and derivative cells thereof using the transduction of polyethylene glycol (PEG) receptors. Also provided are uses of the iPSCs or derivative cells thereof to express a chimeric antigen receptor in combination with a PEG receptor for allogenic cell therapy, and related vectors, polynucleotides, and pharmaceutical compositions.

### REFERENCE TO SEQUENCE LISTING SUBMITTED ELECTRONICALLY

This application contains a sequence listing, which is submitted electronically via EFS-Web as an ASCII formatted sequence listing with a file name “SequenceListing_ST26.xml” and a creation date of Apr. 17, 2023 and having a size of 326 kb. The sequence listing submitted via EFS-Web is part of the specification and is herein incorporated by reference in its entirety.

### BACKGROUND

Data from autologous CAR-T therapy for B cell malignancies have established a strong correlation between (i) cell activation/expansion and (ii) the depth and duration of clinical response. The abundance of targets (e.g., cells that express a CD19 antigen) in the lymphohematopoietic compartment drives activation through the CAR while pre-treatment with lymphodepleting chemotherapy increases the availability of homeostatic cytokines. These two events—signals delivered through the CAR and cytokine receptors—are the initiating drivers necessary for activation, expansion, and ultimately, efficacy of the cell therapy. For many solid tumors, the abundance and accessibility of the target antigen(s) on cancer cells provides insufficient activation signals through the CAR, while the availability of homeostatic cytokines is not enough to support a proliferative response.

Cytokine release syndrome (CRS) and related toxicities are also related to the expansion kinetics of CAR-T cells, and having physician control over expansion kinetics will be critical to the success of cell therapies in solid tumors. However, in order to eliminate the need for lymphodepleting chemotherapy, cell activation/expansion signals must be delivered selectively to the engineered cells in vivo. In other words, expansion of CAR-T in the face of competing endogenous lymphocytes requires targeted delivery of cytokine signals together with CAR driven activation signals. While progress has been made in developing novel forms of exogenous cytokines, these methods require manufacturing novel compounds and co-development. Therefore, there is an unmet need for methods that repurpose clinically approved drugs with known pharmacokinetic properties to drive CAR-driven activation signals together with cytokine signals to achieve controlled cellular expansion and drug exposure.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a schematic of an engineered cell of the present disclosure, which can comprise one or more of a chimeric antigen receptor (CAR), T-Cell Receptor (TCR), and an anti-PEG chimeric receptors (e.g., chimeric activating receptors, and chimeric cytokine receptors). Engineered cells (e.g., iT and/or iNK cells) expressing chimeric receptors specific for PEG may be regulated by systemic and/or local administration of PEG or PEGylated drugs as chimeric receptor crosslinkers. Anti-PEG chimeric receptors can comprise CARs that activate through (i) a TCR-zeta chain and a co-stimulatory domain (e.g., chimeric activating receptors), and/or (ii) the transmembrane and intracellular domains of cytokine receptors (e.g., chimeric cytokine receptors).

FIGS. 2A-B shows flow cytometry data showing successful PEG binding to a PEG-specific chimeric receptor comprising the sequence provided in (A) SEQ ID NO: 178 or (B) SEQ ID NO: 179 expressed on an engineered cell of the present disclosure. A 2A Thy1.1 staining handle was used to identify CAR positive cells, while Qdot655-PEG 2000 MW was used as a staining reagent to identify PEG binding to a PEG-specific chimeric receptor expressed by the engineered cell (e.g., cells expressing chimeric receptors specific for PEG). (A) Flow cytometry data of engineered cells expressing a PEG-specific chimeric receptor comprising an scFv with (5′→3′) a variable heavy chain (VH) and a variable light chain (VL) of an anti-PEG antibody, which indicates 93.6% of cells exhibiting both CAR expression and PEG binding. (B) Flow cytometry data of engineered cells expressing a PEG-specific chimeric receptor comprising an scFv with (5′→3′) a VL and a VH of an anti-PEG antibody, which indicates 88.6% of cells exhibiting both CAR expression and PEG binding.

FIGS. 3A-B shows flow cytometry data showing successful PEG binding to a PEG-specific chimeric receptor comprising the sequence provided in (A) SEQ ID NO: 182 or (B) SEQ ID NO: 180 expressed on an engineered cell of the present disclosure. A 2A Thy1.1 staining handle was used to identify CAR positive cells, while Qdot655-PEG 2000 MW was used as a staining reagent to identify PEG binding to a PEG-specific chimeric receptor expressed by the engineered cell (e.g., cells expressing chimeric receptors specific for PEG). (A) Flow cytometry data of engineered cells expressing a PEG-specific chimeric receptor comprising an scFv with (5′→3′) a variable heavy chain (VH) and a variable light chain (VL) of an anti-PEG antibody. (B) Flow cytometry data of engineered cells expressing a PEG-specific chimeric receptor comprising an scFv with (5′→3′) a VL and a VH of an anti-PEG antibody, which indicates 49.1% of cells exhibiting both CAR expression and PEG binding.

FIGS. 4A-B shows flow cytometry data showing successful PEG binding to a PEG-specific chimeric receptor comprising the sequence provided in (A) SEQ ID NO: 183 or (B) SEQ ID NO: 181 expressed on an engineered cell of the present disclosure. A 2A Thy1.1 staining handle was used to identify CAR positive cells, while Qdot655-PEG 2000 MW was used as a staining reagent to identify PEG binding to a PEG-specific chimeric receptor expressed by the engineered cell (e.g., cells expressing chimeric receptors specific for PEG). (A) Flow cytometry data of engineered cells expressing a PEG-specific chimeric receptor comprising an scFv with (5′→3′) a variable heavy chain (VH) and a variable light chain (VL) of an anti-PEG antibody. (B) Flow cytometry data of engineered cells expressing a PEG-specific chimeric receptor comprising an scFv with (5′→3′) a VL and a VH of an anti-PEG antibody, which indicates 50.6% of cells exhibiting both CAR expression and PEG binding.

FIG. 5 shows flow cytometry data in untransduced cells as a negative control. A 2A Thy1.1 staining handle was used to identify CAR positive cells, while Qdot655-PEG 2000 MW was used as a staining reagent to identify PEG binding to a PEG-specific chimeric receptor expressed by the engineered cell (e.g., cells expressing chimeric receptors specific for PEG).

FIGS. 6A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were then plated in a 96 well plate without Qdot655-PEG2K (ThermoFisher Cat #Q21521MP) to serve as a negative control. All cells were then incubated at 37° C./5% CO2 for 3.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 7A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were then plated in a 96 well plate without Qdot655-PEG2K (ThermoFisher Cat #Q21521MP) to serve as a negative control. All cells were then incubated at 37° C./5% CO2 for 22.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 8A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were then plated in a 96 well plate and co-cultured with Qdot655-PEG2K (ThermoFisher Cat #Q21521MP) that was diluted to 5 nM concentration using sterile cell culturing media. All cells were then incubated at 37° C./5% CO2 for 3.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 9A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were then plated in a 96 well plate and co-cultured with Qdot655-PEG2K (ThermoFisher Cat #Q21521MP) that was diluted to 5 nM concentration using sterile cell culturing media. All cells were then incubated at 37° C./5% CO2 for 22.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 10A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were co-cultured in Immunocult activation reagent diluted in cell culturing media to serve as positive controls. All cells were then incubated at 37° C./5% CO2 for 3.5 hrs. After incubation, cells were stained using fluorescently labeled antibodies specific for Thy1.1 and a viability dye. Using a flow cytometer levels, of GFP expression, Thy1.1 staining, viability and cell size/complexity were measured. Data was analyzed using FlowJo software.

FIGS. 11A-C show Jurkat cells that express green fluorescent protein under the control of the Nur77 promoter, such that Nur77 expression leads to expression of GFP. Cells were transduced with lentivirus encoding anti-PEG CARs. These anti-PEG CAR constructs also include a murine Thy1.1 marker that serves as a proxy for the assessment of the level of CAR expression in a transduced cell. Transduced Jurkat cells were co-cultured in Immunocult activation reagent diluted in cell culturing media to serve as positive controls. All cells were then incubated at 37° C./5% CO2 for 22.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 12A-B show Jurkat cells that express green fluorescent protein under the control of the Nur77 promoter, such that Nur77 expression leads to expression of GFP. Cells were transduced with lentivirus encoding anti-PEG CARs with a short spacer linking the scFv binder to the transmembrane domain. These anti-PEG CAR constructs also include a murine Thy1.1 marker that serves as a proxy for the assessment of the level of CAR expression in a transduced cell. Transduced Jurkat cells were co-cultured with either (A) Qdot655-PEG2K (ThermoFisher Cat #Q21521MP) that was diluted to 10 nM concentration using sterile cell culturing media or (B) cell culturing media alone. All cells were then incubated at 37° C./5% CO2 for approximately 24 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIGS. 13A-C show Jurkat cells that express green fluorescent protein under the control of the Nur77 promoter, such that Nur77 expression leads to expression of GFP. Cells were transduced with lentivirus encoding anti-PEG CARs with a long spacer linking the scFv binder to the transmembrane domain in the case of A-B, or left untransduced in the case of C. These anti-PEG CAR constructs also include a murine Thy1.1 marker that serves as a proxy for the assessment of the level of CAR expression in a transduced cell. Transduced Jurkat cells were co-cultured with either (A) Qdot655-PEG2K (ThermoFisher Cat #Q21521MP) that was diluted to 10 nM concentration using sterile cell culturing media or (B) cell culturing media alone. All cells were then incubated at 37° C./5% CO2 for approximately 24 hrs. After incubation, cells were stained using fluorescently labeled antibodies specific for Thy1.1 and a viability dye. Using a flow cytometer, levels of GFP expression, Thy1.1 staining, viability and cell size/complexity were measured. Data was analyzed using FlowJo software.

FIG. 14 shows quantification of the median fluorescence intensity (MFI) of Nur77 expression in Thy1.1 expressing anti-PEG short spacer or long spacer CAR transduced cells or untransduced control cells co-cultured as described in FIGS. 12-13. Data was analyzed using FlowJo software and graphed using GraphPad Prism software.

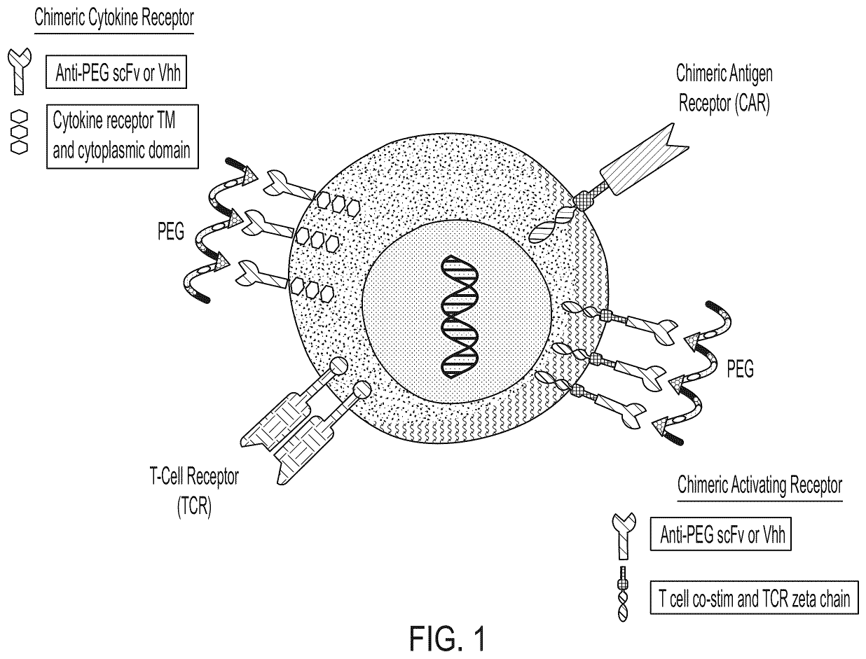

### SUMMARY OF THE INVENTION

In some aspects, the present disclosure provides genetically engineered induced pluripotent stem cells (iPSCs) and/or derivative cells thereof expressing a polyethylene glycol (PEG) receptor. In some aspects, the present disclosure provides methods of using genetically engineered induced pluripotent stem cells (iPSCs) and/or derivative cells thereof expressing a polyethylene glycol (PEG) receptor.

In some embodiments, the engineered cells of the present disclosure express anti-PEG receptors. In some embodiments, the anti-PEG receptors can comprise a polyethylene glycol (PEG) recognition element. In some embodiments, the PEG recognition element can be an scFv. In some embodiments, the PEG recognition element can be a VHH. In some embodiments, the PEG recognition element can be fused to one or more signaling elements to form a chimeric receptor. In some embodiments, the chimeric receptor can be expressed on the surface of the engineered cell.

In some embodiments, an anti-PEG recognition element (e.g., comprising an scFv or VHH) can be fused to one or more of a hinge/spacer, co-stimulatory domain and CD3z chain to form an anti-PEG chimeric antigen receptor (CAR). In other embodiments, a PEG-specific recognition element (e.g., comprising an scFv or VHH) can be fused to one or more of a transmembrane and cytoplasmic domain of a cytokine receptor to form a chimeric cytokine receptor (CCR). In some embodiments, the cytokine receptor comprises IL-7Ra (CD127).

In some aspects, the present disclosure provides methods of transducing induced pluripotent stem cells (iPSCs) and/or derivative cells thereof to express an anti-PEG CARs and/or and anti-PEG CCRs (collectively referred to as anti-PEG chimeric receptors). In some embodiments, the anti-PEG chimeric receptors are expressed on the surface of the engineered cell. In some embodiments, in the presence of PEG, an anti-PEG chimeric receptor can multimerize with adjacent anti-PEG chimeric receptors. In some embodiments, the multimerization of adjacent anti-PEG chimeric receptors can result in signaling to occur or enhance the signal of individual chimeric receptors (e.g., by increasing the avidity of the expressing cell's interaction with repeating PEG units in a polymer).

In some aspects, an engineered cell of the present disclosure comprising a tumor-targeting CAR may be stimulated by administering a PEG-based drug that is recognized by an anti-PEG CAR that is co-expressed by the engineered cell, thereby driving activation and/or expansion of the engineered cell product. In certain embodiments, the PEG-based drug may be administered in vivo or in vitro. For certain in vivo applications, a regulatable cytokine or antigen receptor enables physician-directed control of infused cell product (e.g., proliferation).

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J Mol. Biol. 48:443 (1970), by the search for similarity method of Pearson & Lipman, Proc. Nat'l. Acad. Sci. USA (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “construct” refers to a macromolecule or complex of molecules comprising a polynucleotide to be delivered to a host cell, either in vitro or in vivo, A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably, inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term when used in reference to expression of an encoding nucleic acid refers to expression of an encoding nucleic acid natively contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “engineered immune cell” refers to an immune cell, also referred to as an immune effector cell, that has been genetically modified by the addition of exogenous genetic material in the form of DNA or RNA to the total genetic material of the cell.

### Overview

Provided herein are genetically engineered induced pluripotent stem cells (iPSCs) and derivative cells thereof expressing a polyethylene glycol (PEG) receptors and methods of using the same. In some embodiments, the engineered cells of the present disclosure express anti-PEG receptors comprising a polyethylene glycol (PEG) recognition element (e.g., scFv or VHH) fused to one or more signaling elements to form a receptor that can be expressed on the surface of the cell.

PEG is a highly water-soluble, flexible, uncharged, biocompatible polymer used as an excipient in drug formulation. In some embodiments, a PEG-specific recognition element in the form of an scFv or VHH can be fused to a hinge/spacer, co-stimulatory domain and CD3z chain to form an anti-PEG chimeric antigen receptor (CAR). In other embodiments, a PEG-specific recognition element in the form of an scFv or VHH can be fused to the transmembrane and cytoplasmic domain of a cytokine receptor, for example that of IL-7Ra (CD127), to form a chimeric cytokine receptor (CCR).

Induced pluripotent stem cells (iPSCs) and derivative cells thereof can be transduced to express such anti-PEG CARs and/or anti-PEG CCRs (collectively referred to as anti-PEG chimeric receptors) on the cell's surface. In the presence of PEG, an anti-PEG chimeric receptor can multimerize with adjacent anti-PEG chimeric receptors, causing signaling to occur or enhancing the signal of individual chimeric receptors by increasing the avidity of the expressing cell's interaction with repeating PEG units in a polymer.

Current anti-PEG based therapeutic constructs described in the literature are exclusively acellular (e.g., bispecific antibodies). Using a cell-surface expressed chimeric receptor design allows further control of a drug product's function without relying on a patient's endogenous cells for anti-tumor activity. Using embodiments of the present disclosure, an engineered cell comprising a tumor-targeting CAR may be stimulated by administering a PEG-based drug that is recognized by a co-expressed anti-PEG CAR, thereby driving activation and/or expansion of the engineered cell product. In certain embodiments, the PEG-based drug may be administered in vivo or in vitro. For in vivo applications, a regulatable cytokine or antigen receptor enables physician-directed control of infused cell product (e.g., proliferation).

### Induced Pluripotent Stem Cells (IPSCs) and Immune Effector Cells

IPSCs have unlimited self-renewing capacity. Use of iPSCs enables cellular engineering to produce a controlled cell bank of modified cells that can be expanded and differentiated into desired immune effector cells, supplying large amounts of homogeneous allogeneic therapeutic products.

Provided herein are genetically engineered IPSCs and derivative cells thereof expressing anti-PEG chimeric receptors (e.g., anti-PEG chimeric antigen receptors (CARs) and anti-PEG chimeric cytokine receptors (CCRs). The selected genomic modifications provided herein enhance the therapeutic properties of the derivative cells. The derivative cells are functionally improved and suitable for allogenic off-the-shelf cell therapies following a combination of selective modalities being introduced to the cells at the level of iPSC through genomic engineering. This approach can help to reduce the side effects mediated by CRS/GVHD and prevent long-term autoimmunity while providing excellent efficacy.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the terms “reprogramming” or “dedifferentiation” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” or “HPCs” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). As used herein, “CD34+ hematopoietic progenitor cell” refers to an HPC that expresses CD34 on its surface.

As used herein, the term “immune cell” or “immune effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells), and the like. Additional types of helper T cells include cells such as Th3 (Treg), Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 and CD45 and the absence of the T cell receptor (TCR chains). The NK cell can also refer to a genetically engineered NK cell, such as a NK cell modified to express a chimeric antigen receptor (CAR). The NK cell can also be differentiated from a stem cell or progenitor cell.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that may be used for generating iPSCs through reprogramming, and the source cell derived iPSCs may be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK or “iNK” cells or derivative T or “iT” cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing.

The induced pluripotent stem cell (iPSC) parental cell lines may be generated from peripheral blood mononuclear cells (PBMCs) or T-cells using any known method for introducing re-programming factors into non-pluripotent cells such as the episomal plasmid-based process as previously described in U.S. Pat. Nos. 8,546,140; 9,644,184; 9,328,332; and 8,765,470, the complete disclosures of which are incorporated herein by reference. The reprogramming factors may be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSC's are clonal iPSC's or are obtained from a pool of iPSCs and the genome edits are introduced by making one or more targeted integration and/or in/del at one or more selected sites. In another embodiment, the iPSC's are obtained from human T cells having antigen specificity and a reconstituted TCR gene (hereinafter, also refer to as “T-iPS” cells) as described in U.S. Pat. No. 9,206,394, and 10,787,642 hereby incorporated by reference into the present application.

According to a particular aspect, the application relates to an induced pluripotent stem cell (iPSC) cell or a derivative cell thereof comprising: (i) a first exogenous polynucleotide encoding a chimeric antigen receptor (CAR); (ii) a second exogenous polynucleotide encoding a truncated epithelial growth factor (tEGFR) variant and an interleukin 15 (IL-15), wherein the tEGFR variant and IL-15 are operably linked by an autoprotease peptide sequence, such as the porcine tesehovirus-1 2A (P2A); and (iii) a deletion or reduced expression of B2M and CIITA genes.

### I. Anti-Polyethylene Glycol (PEG) Chimeric Receptor Expression

In certain embodiments of the present disclosure, therapeutic cells can be engineered to comprise a chimeric receptor with a PEG-specific recognition element. In some embodiments, a PEG-specific recognition element in the form of an scFv or VHH can be fused to a hinge/spacer, co-stimulatory domain and CD3z chain to form an anti-PEG chimeric antigen receptor (CAR). In other embodiments, a PEG-specific recognition element in the form of an scFv or VHH can be fused to the transmembrane and cytoplasmic domain of a cytokine receptor (e.g., of IL-7Ra (CD127)) to form a chimeric cytokine receptor (CCR).

In some embodiments, a chimeric receptor can comprise a signal peptide. In some embodiments, an anti-PEG CAR can comprise a signal peptide. In some embodiments, an anti-PEG chimeric cytokine receptor can comprise a signal peptide. Non-limiting examples of signal peptides that may be used with anti-PEG chimeric receptors of the present disclosure are provided in Table 3.

TABLE 3
Signal Peptides for Anti-PEG
Chimeric Receptors

| | | SEQ ID |
|---|---|---|
| CAR regions | Sequence | NO |
| IgK Signal | MARSPAQLLGLLLLWLSGARC | 103 |
| Peptide Variant | | |
| (amino acid) | | |
| IgK Signal | ATGGCCAGATCTCCTGCTCAACTGCT | 144 |
| Peptide Variant | GGGACTGCTGCTGCTGTGGCTTAGCG | |
| (nucleic acid) | GAGCCAGATGC | |
| CD33 Signal | MPLLLLLPLLWAGALA | 145 |
| Peptide | | |
| (amino acid) | | |
| CD33 Signal | ATGCCTTTGCTGCTTCTTCTGCCCCT | 146 |
| Peptide | GCTTTGGGCTGGCGCCCTGGCA | |
| (nucleic acid) | | |

In some embodiments, the signal peptide comprises the amino acid sequence set forth in SEQ ID NO: 103 or 145, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 103 or 145. In some embodiments, the signal peptide is encoded by the nucleic acid sequence set forth in SEQ ID NO: 144 or 146, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 144 or 146.

In some embodiments, a chimeric receptor can comprise an anti-PEG recognition element. In some embodiments, an anti-PEG CAR can comprise an anti-PEG recognition element. In some embodiments, an anti-PEG chimeric cytokine receptor can comprise an anti-PEG recognition element. In some embodiments, an anti-PEG recognition element can comprise one or more scFv domains. In other embodiments, an anti-PEG recognition element can comprise one or more VHH domains. Non-limiting examples of anti-PEG recognition elements that may be used with anti-PEG chimeric receptors of the present disclosure are provided in Table 4.

TABLE 4
Anti-PEG Recognition Elements for Anti-PEG Chimeric Receptors

| | | SEQ ID |
|---|---|---|
| CAR regions | Sequence | NO |
| Anti-PEG scFv | EVKLVESGGGLVQPGGSLKLSCATSGFTFSDYYIYWVRQ | 147 |
| (amino acid) | TPEKRLEWVASISNGGGSTYYPDTLKGRFTISRDSAKNTL | |
| | YLQMSRLKSEDTAMYYCARQHDSSYLAWFAYWGQGTL | |
| | VTVSAGSTSGSGKPGSGEGSDVLMTQTPLSLPVSLGDQAS | |
| | ISCRSSQSIVHSNGNTYLEWYLQKPGQSPKLLIYKVSNRFS | |
| | GVPDRFSGSGSGTDFTLKISRVEAEDLGVYYCFQGSHVPP | |
| | TFGAGTKLELK | |
| Anti-PEG scFv | GAAGTGAAGCTGGTTGAGAGCGGCGGAGGACTGGTGC | 148 |
| (nucleic acid) | AGCCTGGCGGAAGCCTGAAACTGTCTTGCGCCACCAGC | |
| | GGCTTCACCTTTAGCGACTACTACATCTACTGGGTGCG | |
| | GCAGACCCCTGAGAAGCGGCTGGAATGGGTCGCCTCT | |
| | ATCAGCAACGGAGGCGGCAGCACATACTATCCTGATA | |
| | CCCTGAAAGGCAGATTTACCATCAGCCGGGACAGCGC | |
| | CAAGAACACACTGTACCTGCAGATGAGCAGACTGAAA | |
| | AGCGAGGATACAGCCATGTACTACTGCGCCAGACAGC | |
| | ACGACAGCAGCTACCTGGCCTGGTTCGCCTACTGGGGC | |
| | CAGGGCACCCTGGTGACCGTGTCTGCCGGCAGCACCA | |
| | GCGGATCTGGCAAGCCCGGCTCTGGAGAGGGCTCTGA | |
| | TGTGCTGATGACCCAGACACCTCTGAGCCTGCCTGTGT | |
| | CCCTGGGCGACCAGGCCAGCATTAGCTGCAGATCCAG | |
| | CCAGAGCATCGTGCACAGCAATGGCAACACCTACCTG | |
| | GAATGGTACCTGCAAAAGCCTGGCCAATCTCCAAAGCT | |
| | GCTTATCTACAAGGTGTCCAACCGGTTCAGCGGCGTGC | |
| | CCGACAGATTCAGCGGCTCCGGCTCCGGCACAGACTTC | |
| | ACCCTGAAGATCAGTAGAGTGGAAGCCGAGGACCTGG | |
| | GAGTGTACTATTGCTTCCAGGGCTCTCACGTGCCACCT | |
| | ACCTTCGGTGCTGGCACAAAGCTCGAGCTGAAG | |
| Anti-PEG scFv | DVLMTQTPLSLPVSLGDQASISCRSSQSIVHSNGNTYLEW | 149 |
| (amino acid) | YLQKPGQSPKLLIYKVSNRFSGVPDRFSGSGSGTDFTLKIS | |
| | RVEAEDLGVYYCFQGSHVPPTFGAGTKLELKGSTSGSGK | |
| | PGSGEGSEVKLVESGGGLVQPGGSLKLSCATSGFTFSDYY | |
| | IYWVRQTPEKRLEWVASISNGGGSTYYPDTLKGRFTISRD | |
| | SAKNTLYLQMSRLKSEDTAMYYCARQHDSSYLAWFAY | |
| | WGQGTLVTVSA | |
| Anti-PEG scFv | GACGTGCTGATGACCCAGACACCTCTGAGCCTGCCTGT | 150 |
| (nucleic acid) | GTCCCTGGGCGACCAGGCCAGCATCAGCTGTAGAAGC | |
| | AGCCAGAGCATCGTGCACAGCAACGGCAACACCTACC | |
| | TGGAATGGTACCTGCAAAAGCCTGGCCAAAGCCCTAA | |
| | GCTTCTGATCTACAAGGTGTCCAACAGATTCAGCGGAG | |
| | TTCCAGACAGATTTAGCGGCAGCGGTTCCGGCACCGAC | |
| | TTCACCCTGAAAATCTCTAGAGTGGAAGCCGAGGATCT | |
| | GGGCGTGTACTACTGCTTCCAGGGCAGCCACGTGCCCC | |
| | CCACCTTCGGCGCTGGCACAAAGCTCGAGCTGAAAGG | |
| | CTCTACATCCGGCTCCGGCAAGCCCGGCAGCGGCGAG | |
| | GGCTCTGAGGTGAAGCTGGTGGAAAGCGGCGGCGGCC | |
| | TGGTCCAGCCTGGAGGATCTCTGAAGCTGTCCTGCGCT | |
| | ACAAGCGGATTCACCTTTAGCGACTACTACATCTACTG | |
| | GGTGCGGCAGACCCCTGAGAAGCGGCTGGAATGGGTC | |
| | GCCTCTATTTCTAATGGCGGCGGAAGCACATACTATCC | |
| | TGATACCCTGAAGGGCAGATTCACCATCAGCCGCGAC | |
| | AGCGCCAAGAACACACTGTACCTGCAGATGAGCCGGC | |
| | TGAAAAGCGAGGACACCGCCATGTACTATTGCGCCAG | |
| | ACAGCACGATTCTAGCTACCTGGCCTGGTTCGCCTACT | |
| | GGGGCCAGGGCACCCTGGTGACCGTGTCTGCT | |
| Anti-PEG scFv | QIVLTQSPAIMSAFPGERVTLTCSASSSVRSSYLCWYQQK | 151 |
| (amino acid) | PGSSPKLWIYSTSNLASGVPARFSGSGSGTSYSLTISSMEA | |
| | EDAASYFCHQWSSYPRTFGGGTKLEIKGSTSGSGKPGSGE | |
| | GSEVKLEESGGGLVQPGGSMKLSCAASGFIFSDAWMDW | |
| | VRQSPERGLEWVAEIRSKANGLAPYYAESVKGRFTISRD | |
| | DSKSSVYLQMNNLRSEDTGIYYCTSTLYYFDYWGQGTTL | |
| | TVSS | |
| Anti-PEG scFv | CAGATCGTGCTGACCCAGAGCCCAGCAATCATGTCCGC | 152 |
| (nucleic acid) | CTTCCCTGGCGAACGGGTGACACTGACATGCAGCGCCA | |
| | GCTCTAGCGTGCGGAGCAGCTATCTGTGTTGGTACCAA | |
| | CAGAAACCTGGCAGCAGCCCTAAGCTGTGGATCTACA | |
| | GTACCTCCAATCTGGCCTCTGGAGTGCCCGCTAGATTC | |
| | AGCGGATCTGGCTCCGGCACCAGCTACAGCCTGACCAT | |
| | TAGCAGCATGGAAGCCGAGGATGCCGCCAGCTACTTTT | |
| | GCCACCAGTGGAGCTCTTACCCCAGAACATTCGGCGGC | |
| | GGCACAAAGCTGGAAATCAAGGGCAGCACAAGCGGCT | |
| | CAGGCAAGCCCGGCAGCGGCGAGGGCAGCGAGGTGAA | |
| | GCTGGAGGAAAGCGGCGGCGGCCTGGTGCAACCTGGA | |
| | GGAAGCATGAAACTGAGCTGTGCCGCTAGCGGATTTAT | |
| | CTTCTCTGATGCTTGGATGGACTGGGTTCGCCAGTCCC | |
| | CTGAGAGAGGCCTCGAATGGGTGGCCGAGATCAGATC | |
| | CAAGGCCAACGGCCTGGCCCCTTACTACGCCGAGAGC | |
| | GTGAAGGGTAGATTCACCATCAGCCGGGACGACAGCA | |
| | AGTCTTCTGTCTACCTGCAAATGAACAACCTGAGAAGC | |
| | GAGGACACCGGCATCTACTACTGCACCAGCACCCTGTA | |
| | CTACTTCGACTATTGGGGACAGGGCACCACCCTGACAG | |
| | TGTCCTCC | |
| Anti-PEG scFv | QIVLTQSPAIMSAFPGERVTLTCSASSSVRSSYLAWYQQK | 153 |
| (amino acid) | PGSSPKLWIYSTSNLASGVPARFSGSGSGTSYSLTISSMEA | |
| | EDAASYFCHQWSSYPRTFGGGTKLEIKGSTSGSGKPGSGE | |
| | GSEVKLEESGGGLVQPGGSMKLSCAASGFIFSDAWMDW | |
| | VRQSPERGLEWVAEIRSKANGLAPYYAESVKGRFTISRD | |
| | DSKSSVYLQMNNLRSEDTGIYYCTSTLYYFDYWGQGTTL | |
| | TVSS | |
| Anti-PEG scFv | CAGATCGTTCTGACACAGTCCCCAGCTATTATGAGCGC | 154 |
| (nucleic acid) | CTTCCCCGGAGAGCGGGTGACACTGACCTGTAGCGCCT | |
| | CTTCCAGCGTGCGGAGCAGCTATCTGGCCTGGTACCAG | |
| | CAGAAGCCTGGTAGCAGTCCCAAGCTGTGGATCTACA | |
| | GCACCAGCAACCTGGCCTCCGGAGTGCCCGCCAGGTTC | |
| | AGCGGCTCCGGCAGCGGCACAAGCTATAGCCTGACAA | |
| | TCAGCTCCATGGAAGCCGAGGACGCTGCCTCTTACTTC | |
| | TGCCACCAGTGGAGCTCTTACCCTAGAACCTTCGGCGG | |
| | CGGCACCAAGCTGGAAATCAAGGGCTCTACAAGCGGC | |
| | AGCGGAAAACCTGGCAGCGGCGAGGGAAGCGAGGTG | |
| | AAGCTGGAAGAGAGCGGAGGAGGCCTTGTGCAGCCTG | |
| | GCGGCAGCATGAAGCTCAGCTGCGCCGCTTCAGGCTTC | |
| | ATCTTTTCTGATGCCTGGATGGACTGGGTCAGACAGTC | |
| | CCCTGAGAGAGGCCTGGAATGGGTGGCCGAGATCAGA | |
| | AGCAAGGCCAATGGCCTGGCTCCATACTACGCCGAATC | |
| | TGTGAAAGGCAGATTTACCATCTCTCGGGACGACAGCA | |
| | AGAGCAGCGTGTACCTGCAAATGAACAACCTGAGATC | |
| | TGAGGATACAGGCATCTACTACTGCACCAGCACCCTGT | |
| | ACTACTTCGACTACTGGGGCCAAGGCACCACCCTGACC | |
| | GTGTCCTCT | |
| Anti-PEG scFv | EVKLEESGGGLVQPGGSMKLSCAASGFIFSDAWMDWVR | 155 |
| (amino acid) | QSPERGLEWVAEIRSKANGLAPYYAESVKGRFTISRDDSK | |
| | SSVYLQMNNLRSEDTGIYYCTSTLYYFDYWGQGTTLTVS | |
| | SGSTSGSGKPGSGEGSQIVLTQSPAIMSAFPGERVTLTCSA | |
| | SSSVRSSYLCWYQQKPGSSPKLWIYSTSNLASGVPARFSG | |
| | SGSGTSYSLTISSMEAEDAASYFCHQWSSYPRTFGGGTKL | |
| | EIK | |
| Anti-PEG scFv | GAGGTGAAGCTGGAAGAGAGCGGCGGCGGCCTGGTGC | 156 |
| (nucleic acid) | AACCTGGCGGCAGCATGAAGCTGTCATGCGCCGCTTCT | |
| | GGATTTATCTTCAGCGACGCCTGGATGGACTGGGTGCG | |
| | GCAGAGCCCTGAGCGGGGCCTGGAATGGGTCGCCGAG | |
| | ATTAGAAGCAAGGCCAATGGCCTCGCCCCTTACTACGC | |
| | CGAAAGCGTGAAAGGCAGATTCACAATCTCAAGAGAT | |
| | GACAGCAAGAGCAGCGTGTACCTGCAGATGAACAACC | |
| | TGCGGAGCGAGGATACCGGCATCTACTATTGTACCTCT | |
| | ACACTGTACTACTTCGACTACTGGGGCCAGGGCACAAC | |
| | CCTGACCGTGTCCTCTGGATCCACCAGCGGCAGCGGAA | |
| | AACCTGGCAGCGGAGAGGGCAGCCAGATCGTGCTGAC | |
| | ACAGTCCCCCGCTATCATGAGCGCCTTCCCCGGCGAGA | |
| | GAGTGACCCTGACCTGTAGCGCCTCTTCTAGTGTTAGA | |
| | AGCAGTTACCTGTGCTGGTACCAGCAAAAGCCTGGCTC | |
| | TTCTCCAAAGCTGTGGATCTACAGCACCAGCAACCTGG | |
| | CTAGCGGCGTGCCTGCTAGGTTTAGCGGATCCGGCAGC | |
| | GGCACCAGCTACAGCCTGACCATCAGCAGCATGGAAG | |
| | CCGAGGACGCCGCCAGCTATTTCTGCCACCAGTGGTCC | |
| | AGCTACCCCAGAACATTCGGCGGCGGAACCAAGCTGG | |
| | AAATCAAG | |
| Anti-PEG scFv | EVKLEESGGGLVQPGGSMKLSCAASGFIFSDAWMDWVR | 157 |
| (amino acid) | QSPERGLEWVAEIRSKANGLAPYYAESVKGRFTISRDDSK | |
| | SSVYLQMNNLRSEDTGIYYCTSTLYYFDYWGQGTTLTVS | |
| | SGSTSGSGKPGSGEGSQIVLTQSPAIMSAFPGERVTLTCSA | |
| | SSSVRSSYLAWYQQKPGSSPKLWIYSTSNLASGVPARFSG | |
| | SGSGTSYSLTISSMEAEDAASYFCHQWSSYPRTFGGGTKL | |
| | EIK | |
| Anti-PEG scFv | GAAGTGAAGCTGGAAGAGAGCGGAGGCGGCCTGGTGC | 158 |
| (nucleic acid) | AGCCTGGCGGAAGCATGAAACTGTCATGCGCCGCCAG | |
| | CGGCTTCATCTTCAGCGACGCCTGGATGGACTGGGTGC | |
| | GGCAAAGCCCCGAGAGAGGCCTGGAATGGGTCGCCGA | |
| | GATCAGAAGCAAGGCCAACGGCCTGGCCCCTTACTAC | |
| | GCCGAGAGCGTTAAGGGCAGATTCACCATCAGCCGGG | |
| | ACGACTCTAAAAGCAGCGTGTACCTGCAAATGAACAA | |
| | CCTGAGATCCGAGGACACCGGCATCTACTACTGCACCA | |
| | GCACCCTGTACTACTTTGATTACTGGGGCCAGGGCACA | |
| | ACACTGACAGTGTCCTCCGGTTCTACCTCCGGCAGCGG | |
| | CAAGCCCGGCAGCGGCGAGGGCTCTCAGATCGTGCTG | |
| | ACACAGTCCCCAGCCATCATGAGCGCCTTTCCTGGAGA | |
| | AAGAGTGACCCTGACCTGCAGCGCCTCTTCTAGCGTGC | |
| | GGTCCAGCTATCTGGCTTGGTACCAGCAAAAGCCAGGC | |
| | TCTAGCCCTAAGCTGTGGATCTACAGCACATCTAATCT | |
| | GGCCAGCGGCGTGCCTGCTCGGTTCAGCGGCAGCGGC | |
| | AGCGGAACAAGCTACAGCCTGACCATTTCTTCCATGGA | |
| | AGCCGAGGATGCCGCTAGCTACTTCTGCCACCAGTGGT | |
| | CCTCTTATCCTCGTACCTTCGGCGGAGGCACCAAGCTC | |
| | GAGATCAAG | |

In some embodiments, the anti-PEG recognition element comprises the amino acid sequence set forth in SEQ ID NO: 147, 149, 151, 153, 155, or 157, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 147, 149, 151, 153, 155, or 157. In some embodiments, the anti-PEG recognition element is encoded by the nucleic acid sequence set forth in SEQ ID NO: 148, 150, 152, 154, 156, or 158, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 148, 150, 152, 154, 156, or 158.

In some embodiments, a chimeric receptor can comprise a spacer. In some embodiments, an anti-PEG CAR can comprise a spacer. In some embodiments, an anti-PEG chimeric cytokine receptor can comprise a spacer. Non-limiting examples of spacers that may be used with anti-PEG chimeric receptors of the present disclosure are provided in Table 5.

TABLE 5
Spacers for Anti-PEG Chimeric Receptors

| | | SEQ ID |
|---|---|---|
| CAR regions | Sequence | NO |
| Spacer | ESKYGPPCPPCPAPPVAGPSVFLFPPKPKDTLMISRTPEVT | 159 |
| (amino acid) | CVVVDVSQEDPEVQFNWYVDGVEVHNAKTKPREEQFQS | |
| | TYRVVSVLTVLHQDWLNGKEYKCKVSNKGLPSSIEKTIS | |
| | KAKGQPREPQVYTLPPSQEEMTKNQVSLTCLVKGFYPSDI | |
| | AVEWESNGQPENNYKTTPPVLDSDGSFFLYSRLTVDKSR | |
| | WQEGNVFSCSVMHEALHNHYTQKSLSLSLGK | |
| Spacer | GAGTCCAAATACGGTCCGCCATGCCCACCATGCCCAGC | 160 |
| (nucleic acid) | ACCTCCCGTGGCCGGACCATCAGTGTTCCTGTTCCCCC | |
| | CAAAACCCAAGGACACTCTCATGATCTCCCGGACCCCT | |
| | GAGGTCACCTGCGTGGTGGTGGACGTGAGCCAGGAAG | |
| | ATCCCGAGGTCCAGTTCAACTGGTATGTGGATGGCGTG | |
| | GAAGTGCATAATGCCAAGACAAAGCCGCGGGAGGAGC | |
| | AGTTCCAGAGCACGTACCGTGTGGTCAGCGTCCTCACC | |
| | GTCCTGCACCAAGACTGGCTGAACGGCAAGGAGTACA | |
| | AGTGCAAGGTGTCCAACAAAGGCCTCCCGTCCTCCATC | |
| | GAGAAAACCATCTCCAAAGCCAAAGGGCAGCCCCGAG | |
| | AGCCACAGGTGTACACCCTGCCCCCATCCCAAGAGGA | |
| | GATGACCAAGAACCAAGTCAGCCTGACCTGCCTGGTC | |
| | AAAGGCTTCTACCCCAGCGACATCGCCGTGGAGTGGG | |
| | AGAGCAATGGGCAGCCGGAGAACAACTACAAGACCAC | |
| | GCCTCCCGTGCTGGACTCCGACGGCTCCTTCTTCCTCTA | |
| | CTCCCGGCTCACCGTGGACAAGAGCAGGTGGCAGGAG | |
| | GGGAATGTGTTCTCATGCTCCGTGATGCATGAGGCTCT | |
| | GCACAACCACTACACACAGAAGAGCCTCTCCCTGTCTC | |
| | TGGGAAAG | |
| Spacer | SKYGPPCPPCP | 161 |
| (amino acid) | | |
| Spacer | TCCAAATACGGTCCGCCATGCCCACCATGCCCA | 162 |
| (nucleic acid) | | |

In some embodiments, the spacer comprises the amino acid sequence set forth in SEQ ID NO: 159 or 161, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 159 or 161. In some embodiments, the spacer is encoded by the nucleic acid sequence set forth in SEQ ID NO: 160 or 162, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 160 or 162.

In some embodiments, a chimeric receptor can comprise a transmembrane domain. In some embodiments, an anti-PEG CAR can comprise a transmembrane domain. In some embodiments, an anti-PEG chimeric cytokine receptor can comprise a transmembrane domain. Non-limiting examples of transmembrane domains that may be used with anti-PEG chimeric receptors of the present disclosure are provided in Table 6.

TABLE 6
Transmembrane Domains for Anti-PEG Chimeric Receptors

| | | SEQ ID |
|---|---|---|
| CAR regions | Sequence | NO |
| Transmembrane | FWVLVVVGGVLACYSLLVTVAFIIFWV | 24 |
| domain | | |
| (amino acid) | | |
| Transmembrane | TTTTGGGTGCTGGTGGTGGTTGGTGGAGTCCTGGCTTG | 163 |
| domain | CTATTCCTTGCTAGTAACAGTGGCCTTTATTATTTTCTG | |
| (nucleic acid) | GGTG | |
| Transmembrane | PILLTISILSFFSVALLVILACVLW | 164 |
| domain | | |
| (amino acid) | | |
| Transmembrane | CCCATCCTGCTCACCATCAGTATCCTGTCCTTTTTTTCC | 165 |
| domain | GTGGCTCTTCTCGTGATTCTGGCTTGCGTCCTGTGG | |
| (nucleic acid) | | |

In some embodiments, the transmembrane domain comprises the amino acid sequence set forth in SEQ ID NO: 24 or 164, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 24 or 164. In some embodiments, the transmembrane domain is encoded by the nucleic acid sequence set forth in SEQ ID NO: 163 or 165, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 163 or 165.

In some embodiments, a chimeric receptor can comprise a costimulatory domain. In some embodiments, an anti-PEG CAR can comprise a costimulatory domain. Non-limiting examples of costimulatory domains that may be used with anti-PEG chimeric receptors of the present disclosure are provided in Table 7.

TABLE 7
Costimulatory domains for Anti-PEG Chimeric Receptors

| | | SEQ ID |
|---|---|---|
| CAR regions | Sequence | NO |
| Costimulatory | KRGRKKLLYIFKQPFMRPVQTTQEEDGCSCRFPEEEEGGC | 8 |
| domain | EL | |
| (amino acid) | | |
| Costimulatory | AAACGCGGCCGCAAGAAACTCCTGTATATATTCAAAC | 166 |
| domain | AACCATTTATGAGGCCAGTACAAACTACTCAAGAGGA | |
| (nucleic acid) | AGATGGCTGTAGCTGCCGATTTCCAGAAGAAGAAGAA | |
| | GGAGGATGTGAGCTC | |

Any costimulatory domain disclosed herein may be used in a chimeric receptor of the present disclosure. In some embodiments, the costimulatory domain comprises the amino acid sequence set forth in SEQ ID NO: 8, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 8. In some embodiments, the costimulatory domain is encoded by the nucleic acid sequence set forth in SEQ ID NO: 166, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 166.

In some embodiments, a chimeric receptor can comprise an activation domain. In some embodiments, an anti-PEG CAR can comprise an activation domain. Non-limiting examples of activation domains that may be used with anti-PEG chimeric receptors of the present disclosure are provided in Table 8.

TABLE 8
Activation domains for Anti-PEG Chimeric Receptors

| | | SEQ ID |
|---|---|---|
| CAR regions | Sequence | NO |
| Activation/ | RVKFSRSADAPAYQQGQNQLYNELNLGRREEYDVLDKR | 6 |
| Signaling | RGRDPEMGGKPRRKNPQEGLYNELQKDKMAEAYSEIGM | |
| domain | KGERRRGKGHDGLYQGLSTATKDTYDALHMQALPPR | |
| (amino acid) | | |
| Activation/ | AGAGTGAAGTTCAGCAGGAGCGCAGACGCCCCCGCGT | 167 |
| Signaling | ACCAGCAGGGCCAGAACCAGCTCTATAACGAACTCAA | |
| domain | TCTAGGACGAAGAGAGGAGTACGATGTTTTGGACAAG | |
| (nucleic acid) | CGGCGTGGCCGGGACCCTGAGATGGGGGGAAAGCCGA | |
| | GAAGGAAGAACCCTCAGGAAGGCCTGTACAATGAACT | |
| | GCAGAAAGATAAGATGGCGGAGGCCTACAGTGAGATT | |
| | GGGATGAAAGGCGAGCGCCGGAGGGGCAAGGGGCAC | |
| | GATGGCCTTTACCAGGGGCTCAGTACAGCCACCAAGG | |
| | ACACCTACGACGCCCTTCACATGCAGGCCCTGCCCCCT | |
| | CGC | |

Any activation or signaling domain disclosed herein may be used in a chimeric receptor of the present disclosure. In some embodiments, the activation domain comprises the amino acid sequence set forth in SEQ ID NO: 6, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 6. In some embodiments, the activation domain is encoded by the nucleic acid sequence set forth in SEQ ID NO: 167, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 167.

In some embodiments, a chimeric receptor can comprise a cytoplasmic domain. In some embodiments, an anti-PEG CCR can comprise a cytoplasmic domain. Non-limiting examples of cytoplasmic domains that may be used with anti-PEG chimeric receptors of the present disclosure are provided in Table 9.

TABLE 9
Cytoplasmic domains for Anti-PEG Chimeric Receptors

| | | SEQ ID |
|---|---|---|
| CAR regions | Sequence | NO |
| IL7Ra | KKRIKPIVWPSLPDHKKTLEHLCKKPRKNLNVSFNPESFL | 168 |
| Cytoplasmic | DCQIHRVDDIQARDEVEGFLQDTFPQQLEESEKQRLGGD | |
| domain | VQSPNCPSEDVVITPESFGRDSSLTCLAGNVSACDAPILSS | |
| (amino acid) | SRSLDCRESGKNGPHVYQDLLLSLGTTNSTLPPPFSLQSGI | |
| | LTLNPVAQGQPILTSLGSNQEEAYVTMSSFYQNQ | |
| IL7Ra | AAGAAGCGCATCAAGCCCATCGTCTGGCCAAGCCTGC | 169 |
| Cytoplasmic | CCGACCACAAGAAGACCCTCGAGCACCTGTGCAAGAA | |
| domain | ACCGCGAAAGAACCTGAACGTGTCGTTCAACCCGGAG | |
| (nucleic acid) | AGCTTCCTGGACTGTCAAATTCACAGAGTTGATGACAT | |
| | CCAGGCACGCGACGAGGTGGAGGGCTTCCTTCAGGAT | |
| | ACGTTCCCTCAGCAGCTGGAGGAGAGCGAGAAGCAGC | |
| | GGCTCGGGGGTGATGTGCAGAGCCCCAACTGCCCATCC | |
| | GAGGACGTGGTCATCACTCCGGAATCTTTCGGACGGGA | |
| | CAGCTCTCTGACCTGTCTGGCCGGCAACGTGTCCGCGT | |
| | GCGACGCTCCCATACTGAGCTCCTCCCGCTCGCTCGAC | |
| | TGCCGGGAAAGTGGGAAGAATGGCCCTCATGTATATC | |
| | AGGACCTGCTGTTGTCGCTAGGGACGACCAACTCCACC | |
| | CTGCCTCCCCCATTTTCACTGCAATCCGGCATCTTGACA | |
| | CTCAACCCGGTGGCGCAGGGACAGCCGATTCTTACATC | |
| | GCTGGGCTCCAACCAGGAGGAGGCATACGTGACCATG | |
| | TCTAGTTTCTACCAGAACCAA | |

In some embodiments, the cytoplasmic domain comprises the amino acid sequence set forth in SEQ ID NO: 168, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 168. In some embodiments, the cytoplasmic domain is encoded by the nucleic acid sequence set forth in SEQ ID NO: 169, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 169.

In some embodiments, a chimeric receptor can comprise a 2A peptide sequence. In some embodiments, an anti-PEG CAR can comprise a 2A peptide sequence. In some embodiments, an anti-PEG chimeric cytokine receptor can comprise a 2A peptide sequence. Non-limiting examples of 2A peptide sequences that may be used with anti-PEG chimeric receptors of the present disclosure are provided in Table 10.

TABLE 10
2A Peptide Sequences for Anti-PEG Chimeric Receptors

| | | SEQ ID |
|---|---|---|
| CAR regions | Sequence | NO |
| P2A peptide | GSGATNFSLLKQAGDVEENPGP | 170 |
| sequence | | |
| (amino acid) | | |
| P2A peptide | GGATCCGGCGCCACAAACTTCAGCCTGCTGAAACAGG | 171 |
| sequence | CCGGCGACGTGGAGGAAAACCCAGGCCCA | |
| (nucleic acid) | | |
| T2A peptide | GSGEGRGSLLTCGDVEENPGP | 172 |
| sequence | | |
| (amino acid) | | |
| E2A peptide | GSGQCTNYALLKLAGDVESNPGP | 173 |
| sequence | | |
| (amino acid) | | |

In some embodiments, the 2A peptide sequence comprises the amino acid sequence set forth in SEQ ID NO: 170, 172, or 173, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 170, 172, or 173. In some embodiments, the 2A peptide sequence is encoded by the nucleic acid sequence set forth in SEQ ID NO: 171, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 171.

In some embodiments, a chimeric receptor can comprise a staining handle or reporter. In some embodiments, an anti-PEG CAR can comprise a staining handle or reporter. In some embodiments, an anti-PEG chimeric cytokine receptor can comprise a staining handle or reporter. Non-limiting examples of staining handles or reporters that may be used with anti-PEG chimeric receptors of the present disclosure are provided in Table 11.

TABLE 11
Staining Handles/Reporters for Anti-PEG Chimeric Receptors

| | | SEQ ID |
|---|---|---|
| CAR regions | Sequence | NO |
| Murine Thy1.1 | NPAISVALLLSVLQVSRGQKVTSLTACLVNQNLRLDCRH | 174 |
| staining marker | ENNTKDNSIQHEFSLTREKRKHVLSGTLGIPEHTYRSRVT | |
| (amino acid) | LSNQPYIKVLTLANFTTKDEGDYFCELRVSGANPMSSNKS | |
| | ISVYRDKLVKCGGISLLVQNTSWMLLLLLSLSLLQALDFI | |
| | SL | |
| Murine Thy1.1 | AACCCAGCCATCAGCGTCGCTCTCCTGCTCTCAGTCTT | 175 |
| staining marker | GCAAGTGTCCCGAGGGCAGAAAGTGACCAGCCTGACA | |
| (nucleic acid) | GCCTGCCTGGTCAACCAGAACCTGAGACTGGACTGCCG | |
| | GCACGAGAACAACACCAAGGACAACAGCATCCAGCAC | |
| | GAGTTCAGCCTGACCAGAGAAAAGCGGAAACACGTGC | |
| | TGAGCGGCACCCTGGGAATCCCCGAGCACACCTATAG | |
| | AAGCAGAGTGACCCTGAGCAACCAGCCTTACATCAAA | |
| | GTGCTGACCCTGGCCAACTTCACCACCAAGGATGAGG | |
| | GCGACTACTTCTGCGAGCTGAGAGTGTCTGGCGCCAAT | |
| | CCTATGAGCAGCAACAAGAGCATCAGCGTGTACCGGG | |
| | ACAAGCTGGTCAAGTGTGGCGGCATCTCTCTGCTGGTG | |
| | CAGAACACCTCTTGGATGCTGCTGCTCCTGCTGAGCCT | |
| | GAGTCTGCTGCAAGCCCTGGATTTCATCAGCCTG | |
| GFP Reporter | MVSKGEELFTGVVPILVELDGDVNGHKFSVRGEGEGDAT | 176 |
| (amino acid) | NGKLTLKFICTTGKLPVPWPTLVTTLTYGVQCFSRYPDH | |
| | MKQHDFFKSAMPEGYVQERTITFKDDGTYKTRAEVKFE | |
| | GDTLVNRIELKGIDFKEDGNILGHKLEYNFNSHNVYITAD | |
| | KQKNGIKANFKIRHNVEDGSVQLADHYQQNTPIGDGPVL | |
| | LPDNHYLSTQSKLSKDPNEKRDHMVLLEFVTAAGITHGM | |
| | DELYK | |
| GFP Reporter | ATGGTGTCCAAGGGCGAAGAACTGTTCACCGGCGTGG | 177 |
| (nucleic acid) | TGCCCATTCTGGTGGAACTGGACGGGGATGTGAACGG | |
| | CCACAAGTTCAGCGTTAGAGGCGAAGGCGAAGGGGAT | |
| | GCCACAAACGGCAAGCTGACCCTGAAGTTCATCTGCAC | |
| | CACCGGAAAGCTGCCCGTGCCTTGGCCTACACTGGTCA | |
| | CCACACTGACATACGGCGTGCAGTGCTTCAGCAGATAC | |
| | CCCGACCATATGAAGCAGCACGACTTCTTCAAGAGCGC | |
| | CATGCCTGAGGGCTACGTGCAAGAGAGAACCATCACC | |
| | TTCAAGGACGACGGCACCTACAAGACCAGAGCCGAAG | |
| | TGAAGTTCGAGGGCGACACCCTGGTCAACCGGATCGA | |
| | GCTGAAGGGCATCGACTTCAAAGAGGACGGCAACATC | |
| | CTGGGCCACAAACTTGAGTACAACTTCAACAGCCACA | |
| | ACGTGTAtATCACCGCCGACAAGCAGAAGAACGGCATC | |
| | AAGGCCAACTTCAAGATCCGGCACAACGTGGAAGATG | |
| | GCAGCGTGCAGCTGGCCGATCACTACCAGCAGAACAC | |
| | ACCCATCGGAGATGGCCCTGTGCTGCTGCCCGATAACC | |
| | ACTACCTGAGCACCCAGAGCAAGCTGAGCAAGGACCC | |
| | CAACGAGAAGCGGGACCACATGGTGCTGCTGGAATTT | |
| | GTGACAGCCGCCGGAATCACCCACGGCATGGATGAGC | |
| | TGTACAAG | |

In some embodiments, the staining handle or reporter comprises the amino acid sequence set forth in SEQ ID NO:174 or 176, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 174 or 176. In some embodiments, the staining handle or reporter is encoded by the nucleic acid sequence set forth in SEQ ID NO: 175 or 177, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 175 or 177.

In some embodiments, a therapeutic or engineered cell of the present disclosure can comprise one or more chimeric receptors. In some embodiments, the chimeric receptor comprises an anti-PEG CAR. In some embodiments, the chimeric receptor comprises an anti-PEG CCR. In some embodiments, a therapeutic or engineered cell of the present disclosure can comprise both an anti-PEG CAR and an anti-PEG CCR. Non-limiting examples of chimeric receptors that may be expressed by therapeutic or engineered cells of the present disclosure are provided in Table 12.

TABLE 12
Anti-PEG Chimeric Receptors

| | | SEQ ID |
|---|---|---|
| CAR regions | Sequence | NO |
| Anti-PEG | MARSPAQLLGLLLLWLSGARCEVKLVESGGGLVQPGGSL | 178 |
| CAR 1 | KLSCATSGFTFSDYYIYWVRQTPEKRLEWVASISNGGGST | |
| | YYPDTLKGRFTISRDSAKNTLYLQMSRLKSEDTAMYYCA | |
| | RQHDSSYLAWFAYWGQGTLVTVSAGSTSGSGKPGSGEG | |
| | SDVLMTQTPLSLPVSLGDQASISCRSSQSIVHSNGNTYLE | |
| | WYLQKPGQSPKLLIYKVSNRFSGVPDRFSGSGSGTDFTLK | |
| | ISRVEAEDLGVYYCFQGSHVPPTFGAGTKLELKESKYGPP | |
| | CPPCPAPPVAGPSVFLFPPKPKDTLMISRTPEVTCVVVDVS | |
| | QEDPEVQFNWYVDGVEVHNAKTKPREEQFQSTYRVVSV | |
| | LTVLHQDWLNGKEYKCKVSNKGLPSSIEKTISKAKGQPR | |
| | EPQVYTLPPSQEEMTKNQVSLTCLVKGFYPSDIAVEWES | |
| | NGQPENNYKTTPPVLDSDGSFFLYSRLTVDKSRWQEGNV | |
| | FSCSVMHEALHNHYTQKSLSLSLGKFWVLVVVGGVLAC | |
| | YSLLVTVAFIIFWVKRGRKKLLYIFKQPFMRPVQTTQEED | |
| | GCSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPRGSGATNFSLLKQAGDVEENPGP | |
| Anti-PEG | MARSPAQLLGLLLLWLSGARCDVLMTQTPLSLPVSLGDQ | 179 |
| CAR 2 | ASISCRSSQSIVHSNGNTYLEWYLQKPGQSPKLLIYKVSN | |
| | RFSGVPDRFSGSGSGTDFTLKISRVEAEDLGVYYCFQGSH | |
| | VPPTFGAGTKLELKGSTSGSGKPGSGEGSEVKLVESGGGL | |
| | VQPGGSLKLSCATSGFTFSDYYIYWVRQTPEKRLEWVASI | |
| | SNGGGSTYYPDTLKGRFTISRDSAKNTLYLQMSRLKSEDT | |
| | AMYYCARQHDSSYLAWFAYWGQGTLVTVSAESKYGPP | |
| | CPPCPAPPVAGPSVFLFPPKPKDTLMISRTPEVTCVVVDVS | |
| | QEDPEVQFNWYVDGVEVHNAKTKPREEQFQSTYRVVSV | |
| | LTVLHQDWLNGKEYKCKVSNKGLPSSIEKTISKAKGQPR | |
| | EPQVYTLPPSQEEMTKNQVSLTCLVKGFYPSDIAVEWES | |
| | NGQPENNYKTTPPVLDSDGSFFLYSRLTVDKSRWQEGNV | |
| | FSCSVMHEALHNHYTQKSLSLSLGKFWVLVVVGGVLAC | |
| | YSLLVTVAFIIFWVKRGRKKLLYIFKQPFMRPVQTTQEED | |
| | GCSCRFPEEEEGGCELRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPRGSGATNFSLLKQAGDVEENPGP | |
| Anti-PEG | MARSPAQLLGLLLLWLSGARCQIVLTQSPAIMSAFPGERV | 180 |
| CAR 3 | TLTCSASSSVRSSYLCWYQQKPGSSPKLWIYSTSNLASGV | |
| | PARFSGSGSGTSYSLTISSMEAEDAASYFCHQWSSYPRTF | |
| | GGGTKLEIKGSTSGSGKPGSGEGSEVKLEESGGGLVQPGG | |
| | SMKLSCAASGFIFSDAWMDWVRQSPERGLEWVAEIRSK | |
| | ANGLAPYYAESVKGRFTISRDDSKSSVYLQMNNLRSEDT | |
| | GIYYCTSTLYYFDYWGQGTTLTVSSESKYGPPCPPCPAPP | |
| | VAGPSVFLFPPKPKDTLMISRTPEVTCVVVDVSQEDPEVQ | |
| | FNWYVDGVEVHNAKTKPREEQFQSTYRVVSVLTVLHQD | |
| | WLNGKEYKCKVSNKGLPSSIEKTISKAKGQPREPQVYTLP | |
| | PSQEEMTKNQVSLTCLVKGFYPSDIAVEWESNGQPENNY | |
| | KTTPPVLDSDGSFFLYSRLTVDKSRWQEGNVFSCSVMHE | |
| | ALHNHYTQKSLSLSLGKFWVLVVVGGVLACYSLLVTVA | |
| | FIIFWVKRGRKKLLYIFKQPFMRPVQTTQEEDGCSCRFPE | |
| | EEEGGCELRVKFSRSADAPAYQQGQNQLYNELNLGRREE | |
| | YDVLDKRRGRDPEMGGKPRRKNPQEGLYNELQKDKMA | |
| | EAYSEIGMKGERRRGKGHDGLYQGLSTATKDTYDALHM | |
| | QALPPRGSGATNFSLLKQAGDVEENPGP | |
| Anti-PEG | MARSPAQLLGLLLLWLSGARCQIVLTQSPAIMSAFPGERV | 181 |
| CAR 4 | TLTCSASSSVRSSYLAWYQQKPGSSPKLWIYSTSNLASGV | |
| | PARFSGSGSGTSYSLTISSMEAEDAASYFCHQWSSYPRTF | |
| | GGGTKLEIKGSTSGSGKPGSGEGSEVKLEESGGGLVQPGG | |
| | SMKLSCAASGFIFSDAWMDWVRQSPERGLEWVAEIRSK | |
| | ANGLAPYYAESVKGRFTISRDDSKSSVYLQMNNLRSEDT | |
| | GIYYCTSTLYYFDYWGQGTTLTVSSESKYGPPCPPCPAPP | |
| | VAGPSVFLFPPKPKDTLMISRTPEVTCVVVDVSQEDPEVQ | |
| | FNWYVDGVEVHNAKTKPREEQFQSTYRVVSVLTVLHQD | |
| | WLNGKEYKCKVSNKGLPSSIEKTISKAKGQPREPQVYTLP | |
| | PSQEEMTKNQVSLTCLVKGFYPSDIAVEWESNGQPENNY | |
| | KTTPPVLDSDGSFFLYSRLTVDKSRWQEGNVFSCSVMHE | |
| | ALHNHYTQKSLSLSLGKFWVLVVVGGVLACYSLLVTVA | |
| | FIIFWVKRGRKKLLYIFKQPFMRPVQTTQEEDGCSCRFPE | |
| | EEEGGCELRVKFSRSADAPAYQQGQNQLYNELNLGRREE | |
| | YDVLDKRRGRDPEMGGKPRRKNPQEGLYNELQKDKMA | |
| | EAYSEIGMKGERRRGKGHDGLYQGLSTATKDTYDALHM | |
| | QALPPRGSGATNFSLLKQAGDVEENPGP | |
| Anti-PEG | MARSPAQLLGLLLLWLSGARCEVKLEESGGGLVQPGGS | 182 |
| CAR 5 | MKLSCAASGFIFSDAWMDWVRQSPERGLEWVAEIRSKA | |
| | NGLAPYYAESVKGRFTISRDDSKSSVYLQMNNLRSEDTGI | |
| | YYCTSTLYYFDYWGQGTTLTVSSGSTSGSGKPGSGEGSQI | |
| | VLTQSPAIMSAFPGERVTLTCSASSSVRSSYLCWYQQKPG | |
| | SSPKLWIYSTSNLASGVPARFSGSGSGTSYSLTISSMEAED | |
| | AASYFCHQWSSYPRTFGGGTKLEIKESKYGPPCPPCPAPP | |
| | VAGPSVFLFPPKPKDTLMISRTPEVTCVVVDVSQEDPEVQ | |
| | FNWYVDGVEVHNAKTKPREEQFQSTYRVVSVLTVLHQD | |
| | WLNGKEYKCKVSNKGLPSSIEKTISKAKGQPREPQVYTLP | |
| | PSQEEMTKNQVSLTCLVKGFYPSDIAVEWESNGQPENNY | |
| | KTTPPVLDSDGSFFLYSRLTVDKSRWQEGNVFSCSVMHE | |
| | ALHNHYTQKSLSLSLGKFWVLVVVGGVLACYSLLVTVA | |
| | FIIFWVKRGRKKLLYIFKQPFMRPVQTTQEEDGCSCRFPE | |
| | EEEGGCELRVKFSRSADAPAYQQGQNQLYNELNLGRREE | |
| | YDVLDKRRGRDPEMGGKPRRKNPQEGLYNELQKDKMA | |
| | EAYSEIGMKGERRRGKGHDGLYQGLSTATKDTYDALHM | |
| | QALPPRGSGATNFSLLKQAGDVEENPGP | |
| Anti-PEG | MARSPAQLLGLLLLWLSGARCEVKLEESGGGLVQPGGS | 183 |
| CAR 6 | MKLSCAASGFIFSDAWMDWVRQSPERGLEWVAEIRSKA | |
| | NGLAPYYAESVKGRFTISRDDSKSSVYLQMNNLRSEDTGI | |
| | YYCTSTLYYFDYWGQGTTLTVSSGSTSGSGKPGSGEGSQI | |
| | VLTQSPAIMSAFPGERVTLTCSASSSVRSSYLAWYQQKPG | |
| | SSPKLWIYSTSNLASGVPARFSGSGSGTSYSLTISSMEAED | |
| | AASYFCHQWSSYPRTFGGGTKLEIKESKYGPPCPPCPAPP | |
| | VAGPSVFLFPPKPKDTLMISRTPEVTCVVVDVSQEDPEVQ | |
| | FNWYVDGVEVHNAKTKPREEQFQSTYRVVSVLTVLHQD | |
| | WLNGKEYKCKVSNKGLPSSIEKTISKAKGQPREPQVYTLP | |
| | PSQEEMTKNQVSLTCLVKGFYPSDIAVEWESNGQPENNY | |
| | KTTPPVLDSDGSFFLYSRLTVDKSRWQEGNVFSCSVMHE | |
| | ALHNHYTQKSLSLSLGKFWVLVVVGGVLACYSLLVTVA | |
| | FIIFWVKRGRKKLLYIFKQPFMRPVQTTQEEDGCSCRFPE | |
| | EEEGGCELRVKFSRSADAPAYQQGQNQLYNELNLGRREE | |
| | YDVLDKRRGRDPEMGGKPRRKNPQEGLYNELQKDKMA | |
| | EAYSEIGMKGERRRGKGHDGLYQGLSTATKDTYDALHM | |
| | QALPPRGSGATNFSLLKQAGDVEENPGP | |
| Anti-PEG | MARSPAQLLGLLLLWLSGARCEVKLVESGGGLVQPGGSL | 184 |
| CAR 7 | KLSCATSGFTFSDYYIYWVRQTPEKRLEWVASISNGGGST | |
| | YYPDTLKGRFTISRDSAKNTLYLQMSRLKSEDTAMYYCA | |
| | RQHDSSYLAWFAYWGQGTLVTVSAGSTSGSGKPGSGEG | |
| | SDVLMTQTPLSLPVSLGDQASISCRSSQSIVHSNGNTYLE | |
| | WYLQKPGQSPKLLIYKVSNRFSGVPDRFSGSGSGTDFTLK | |
| | ISR VEAEDLGVYYCFQGSHVPPTFGAGTKLELKSKYGPPC | |
| | PPCPFWVLVVVGGVLACYSLLVTVAFIIFWVKRGRKKLL | |
| | YIFKQPFMRPVQTTQEEDGCSCRFPEEEEGGCELRVKFSR | |
| | SADAPAYQQGQNQLYNELNLGRREEYDVLDKRRGRDPE | |
| | MGGKPRRKNPQEGLYNELQKDKMAEAYSEIGMKGERRR | |
| | GKGHDGLYQGLSTATKDTYDALHMQALPPRGSGATNFS | |
| | LLKQAGDVEENPGP | |
| Anti-PEG | MPLLLLLPLLWAGALAEVKLVESGGGLVQPGGSLKLSCA | 185 |
| CCR 1 | TSGFTFSDYYIYWVRQTPEKRLEWVASISNGGGSTYYPDT | |
| | LKGRFTISRDSAKNTLYLQMSRLKSEDTAMYYCARQHDS | |
| | SYLAWFAYWGQGTLVTVSAGSTSGSGKPGSGEGSDVLM | |
| | TQTPLSLPVSLGDQASISCRSSQSIVHSNGNTYLEWYLQK | |
| | PGQSPKLLIYKVSNRFSGVPDRFSGSGSGTDFTLKISRVEA | |
| | EDLGVYYCFQGSHVPPTFGAGTKLELKSKYGPPCPPCPPI | |
| | LLTISILSFFSVALLVILACVLWKKRIKPIVWPSLPDHKKTL | |
| | EHLCKKPRKNLNVSFNPESFLDCQIHRVDDIQARDEVEGF | |
| | LQDTFPQQLEESEKQRLGGDVQSPNCPSEDVVITPESFGR | |
| | DSSLTCLAGNVSACDAPILSSSRSLDCRESGKNGPHVYQD | |
| | LLLSLGTTNSTLPPPFSLQSGILTLNPVAQGQPILTSLGSNQ | |
| | EEAYVTMSSFYQNQGSGATNFSLLKQAGDVEENPGP | |
| Anti-PEG | MPLLLLLPLLWAGALAEVKLVESGGGLVQPGGSLKLSCA | 186 |
| CCR 2 | TSGFTFSDYYIYWVRQTPEKRLEWVASISNGGGSTYYPDT | |
| | LKGRFTISRDSAKNTLYLQMSRLKSEDTAMYYCARQHDS | |
| | SYLAWFAYWGQGTLVTVSAGSTSGSGKPGSGEGSDVLM | |
| | TQTPLSLPVSLGDQASISCRSSQSIVHSNGNTYLEWYLQK | |
| | PGQSPKLLIYKVSNRFSGVPDRFSGSGSGTDFTLKISRVEA | |
| | EDLGVYYCFQGSHVPPTFGAGTKLELKESKYGPPCPPCPA | |
| | PPVAGPSVFLFPPKPKDTLMISRTPEVTCVVVDVSQEDPE | |
| | VQFNWYVDGVEVHNAKTKPREEQFQSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGKPILLTISILSFFSVALLVILAC | |
| | VLWKKRIKPIVWPSLPDHKKTLEHLCKKPRKNLNVSFNP | |
| | ESFLDCQIHRVDDIQARDEVEGFLQDTFPQQLEESEKQRL | |
| | GGDVQSPNCPSEDVVITPESFGRDSSLTCLAGNVSACDAP | |
| | ILSSSRSLDCRESGKNGPHVYQDLLLSLGTTNSTLPPPFSL | |
| | QSGILTLNPVAQGQPILTSLGSNQEEAYVTMSSFYQNQGS | |
| | GATNFSLLKQAGDVEENPGP | |

In some embodiments, a therapeutic of engineered cell of the present disclosure can comprise one or more anti-PEG chimeric receptors comprising the amino acid sequence set forth in SEQ ID NO: 178-186, or a variant thereof having at least 50%, at least 55%, at least 60%, at least 65%, at least 70%, at least 75%, at least 80%, at least 85%, at least 90%, at least 95%, at least 96%, at least 97%, at least 98%, or at least 99%, sequence identity with SEQ ID NO: 178-186.

### II. Chimeric Antigen Receptor (CAR) Expression

According to embodiments of the application, an iPSC cell or a derivative cell thereof can comprise one or more first exogenous polynucleotides encoding a first and a second chimeric antigen receptor (CAR), such as a CAR targeting one or more tumor antigens. In one embodiment, the first CAR targets a CD19 antigen, and the second CAR targets a CD22 antigen. In another embodiment, the first CAR targets a CD19 antigen, and the second CAR targets a CD22 antigen, and the targeting regions (e.g., the extracellular domains) of one or both of the CARs comprises an antibody fragment (e.g., a VHH domain).

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen-binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., NK cell or T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen-binding domain and/or an antigen-binding fragment. The antigen-binding fragment can, for example, be an antibody or antigen-binding fragment thereof that specifically binds a tumor antigen. The antigen-binding fragments of the application possess one or more desirable functional properties, including but not limited to high-affinity binding to a tumor antigen, high specificity to a tumor antigen, the ability to stimulate complement-dependent cytotoxicity (CDC), antibody-dependent phagocytosis (ADPC), and/or antibody-dependent cellular-mediated cytotoxicity (ADCC) against cells expressing a tumor antigen, and the ability to inhibit tumor growth in subjects in need thereof and in animal models when administered alone or in combination with other anti-cancer therapies.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a VHH having binding specificity for a first epitope, and a VHH having binding specificity for a second epitope. In an embodiment, the term X/Y loop (wherein ‘X’ and ‘Y’ are antigens such as CD19 and CD22) refers to an extracellular region in which one scFv (either CD19 or CD22) is nested in between the VL and VH of the other scFv. In some embodiments, X and Y may be the same antigen. In some embodiments, X and Y may be different antigens. In some embodiments, X and Y are tumor antigens.

As used herein, an antigen-binding domain or antigen-binding fragment that “specifically binds to a tumor antigen” refers to an antigen-binding domain or antigen-binding fragment that binds a tumor antigen, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen-binding domain or antigen-binding fragment can be determined by using surface plasmon resonance, such as by using a biosensor system, e.g., a Biacore® system, or by using bio-layer interferometry technology, such as an Octet RED96 system.

The smaller the value of the KD of an antigen-binding domain or antigen-binding fragment, the higher affinity that the antigen-binding domain or antigen-binding fragment binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment. In some embodiments, the antigen-binding fragment is a VHH.

In some embodiments, at least one of the extracellular tag-binding domain, the antigen-binding domain, or the tag comprises a single-domain antibody or nanobody. In some embodiments, at least one of the extracellular tag-binding domain, the antigen-binding domain, or the tag comprises a VHH.

In some embodiments, the extracellular tag-binding domain and the tag each comprise a VHH.

In some embodiments, the extracellular tag-binding domain, the tag, and the antigen-binding domain each comprise a VHH.

In some embodiments, at least one of the extracellular tag-binding domain, the antigen-binding domain, or the tag comprises an scFv.

In some embodiments, the extracellular tag-binding domain and the tag each comprise an scFv.

In some embodiments, the extracellular tag-binding domain, the tag, and the antigen-binding domain each comprise a scFv.

Alternative scaffolds to immunoglobulin domains that exhibit similar functional characteristics, such as high-affinity and specific binding of target biomolecules, may also be used in the CARs of the present disclosure. Such scaffolds have been shown to yield molecules with improved characteristics, such as greater stability or reduced immunogenicity. Non-limiting examples of alternative scaffolds that may be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, fibronectin type III (10Fn3) domain (e.g., monobodies, AdNectins™, or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD3 (e.g., Tetranectin); thioredoxin (e.g., peptide aptamer); KALBITOR®; the (3-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biolechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety).

In some embodiments, the alternative scaffold is Affilin or Centyrin.

In some embodiments, the first polypeptide of the CARs of the present disclosure comprises a leader sequence. The leader sequence may be positioned at the N-terminus the extracellular tag-binding domain. The leader sequence may be optionally cleaved from the extracellular tag-binding domain during cellular processing and localization of the CAR to the cellular membrane. Any of various leader sequences known to one of skill in the art may be used as the leader sequence. Non-limiting examples of peptides from which the leader sequence may be derived include granulocyte-macrophage colony-stimulating factor receptor (GMCSFR), FcεR, human immunoglobulin (IgG) heavy chain (HC) variable region, CD8α, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 1, or a variant thereof having at least 50, at least 55, at least 60, at least at least 70, at least 75, at least 80, at least 85, at least 90, at least 95, at least 96, at least 97, at least 98 or at least 99%, sequence identity with SEQ ID NO: 1.

In some embodiments, the first polypeptide of the CARs of the present disclosure comprise a transmembrane domain, fused in frame between the extracellular tag-binding domain and the cytoplasmic domain.

The transmembrane domain may be derived from the protein contributing to the extracellular tag-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor (TCR), CD28, CD3 epsilon, CD45, CD4, CD5, CD8, CD8a, CD9, CD16, CD22, CD28, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the CAR of the present disclosure comprises a spacer region between the extracellular tag-binding domain and the transmembrane domain, wherein the tag-binding domain, linker, and the transmembrane domain are in frame with each other.

The term “spacer region” as used herein generally means any oligo- or polypeptide that functions to link the tag-binding domain to the transmembrane domain. A spacer region can be used to provide more flexibility and accessibility for the tag-binding domain. A spacer region may comprise up to 300 amino acids, preferably 10 to 100 amino acids and most preferably 25 to 50 amino acids. A spacer region may be derived from all or part of naturally occurring molecules, such as from all or part of the extracellular region of CD8, CD4 or CD28, or from all or part of an antibody constant region. Alternatively, the spacer region may be a synthetic sequence that corresponds to a naturally occurring spacer region sequence, or may be an entirely synthetic spacer region sequence. Non-limiting examples of spacer regions which may be used in accordance to the disclosure include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer may be mutated to prevent Fc receptor binding.

In some embodiments, the spacer region comprises a hinge domain. The hinge domain may be derived from CD8, CD8a, CD28, or an immunoglobulin (IgG). For example, the IgG hinge may be from IgG1, IgG2, IgG3, IgG4, IgG4 CH3, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgG4 CH3, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 57) from IgG1, ERKCCVECPPCP (SEQ ID NO: 58) from IgG2, ELKTPLGDTTHTCPRCP(EPKSCDTPPPCPRCP) 3 (SEQ ID NO: 59) from IgG3, ESKYGPPCPSCP (SEQ ID NO: 60) from IgG4 (see also Wypych et al., JBC 2008 283(23): 16194-16205, which is incorporated herein by reference in its entirety for all purposes), and ESKYGPPCPPCPGQPREPQVYTLPPSQEEMTKNQVSLTCLVKGFYP SDIAVEWES NGQPENNYKTTPPVLDSDGSFFLYSRLTVDKSRWQEGNVFSCSVMHEALHNHY TQKSLSLSLGK (SEQ ID NO: 102), or a variant thereof having at least 50, at least 55, at least 60, at least 65, at least 70, at least 75, at least 80, at least 85, at least 90, at least 95, at least 96, at least 97, at least 98 or at least 99%, sequence identity. In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

In certain aspects, the first polypeptide of CARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD5, CD22, CD226, CD66d, CD79A, and CD79B.

In some embodiments, the cytoplasmic domain comprises a CD3ζ signaling domain. In one embodiment, the CD3ζ signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set forth in SEQ ID NO: 10, or a variant thereof having at least 50, at least 55, at least 60, at least 65, at least 70, at least 75, at least 80, at least 85, at least 90, at least at least 96, at least 97, at least 98 or at least 99%, sequence identity with SEQ ID NO: In one embodiment, the co-stimulatory signaling domain is derived from OX40.

In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least at least 96, at least 97, at least 98 or at least 99%, sequence identity with SEQ ID NO:

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

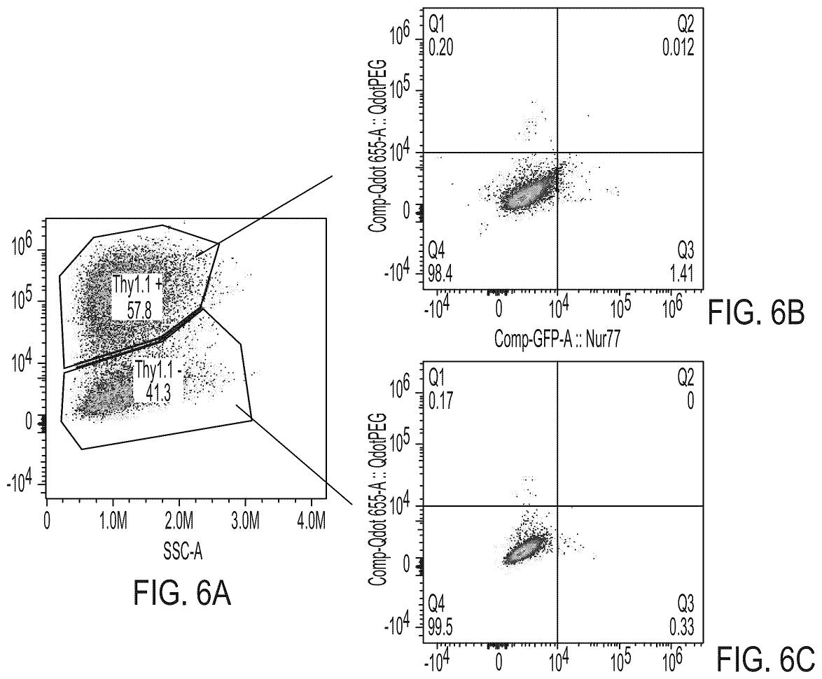

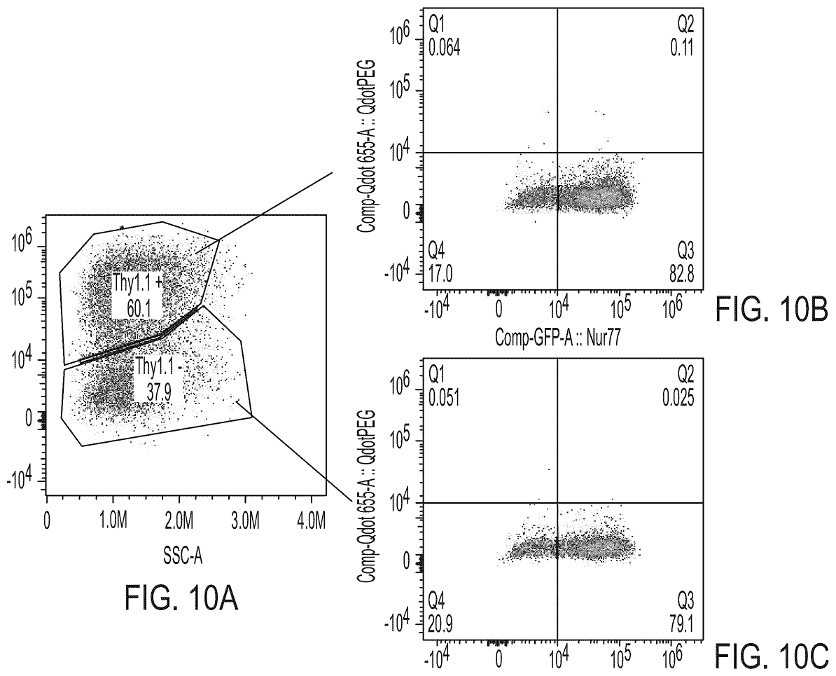

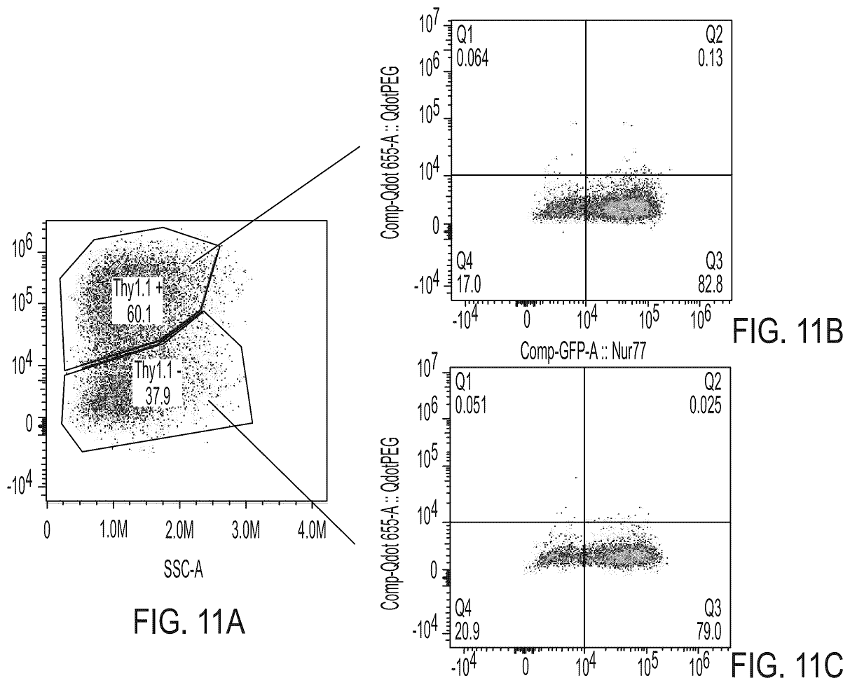

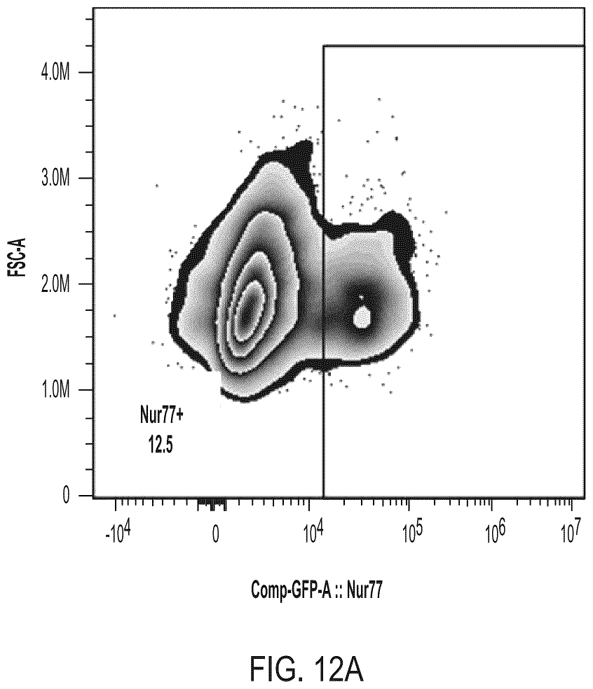

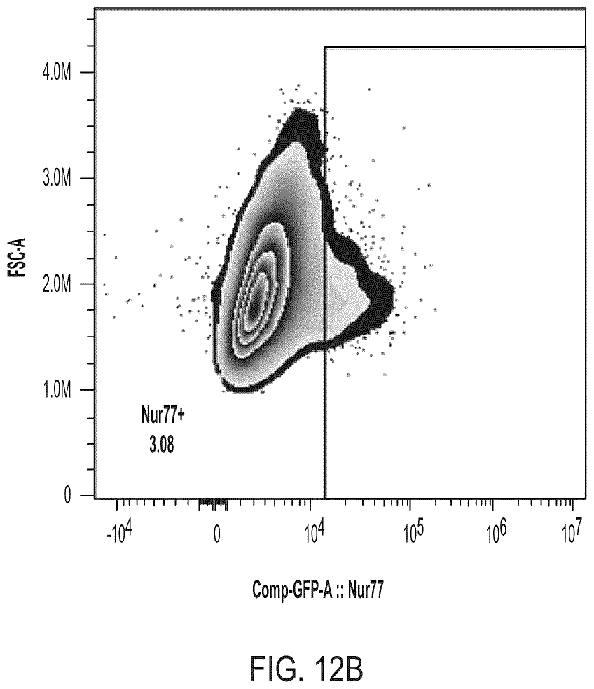

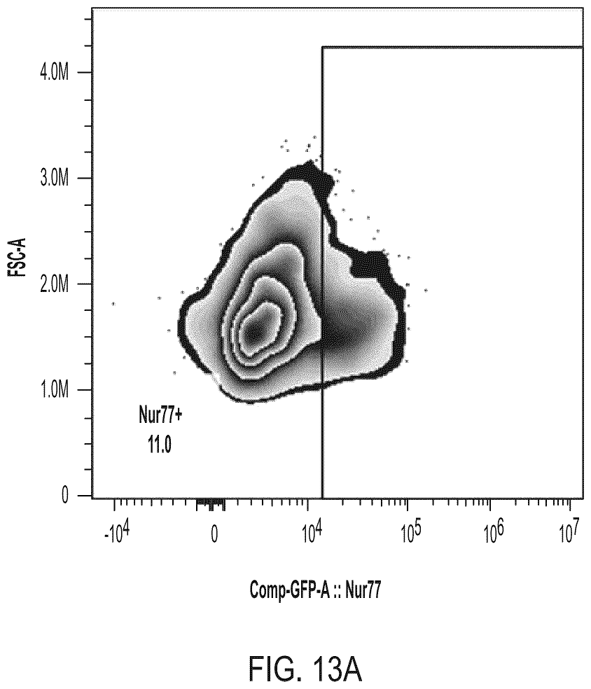

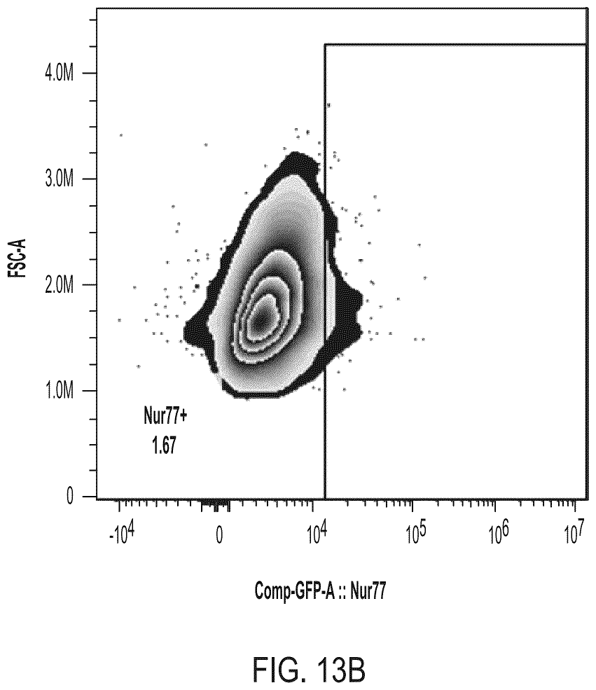

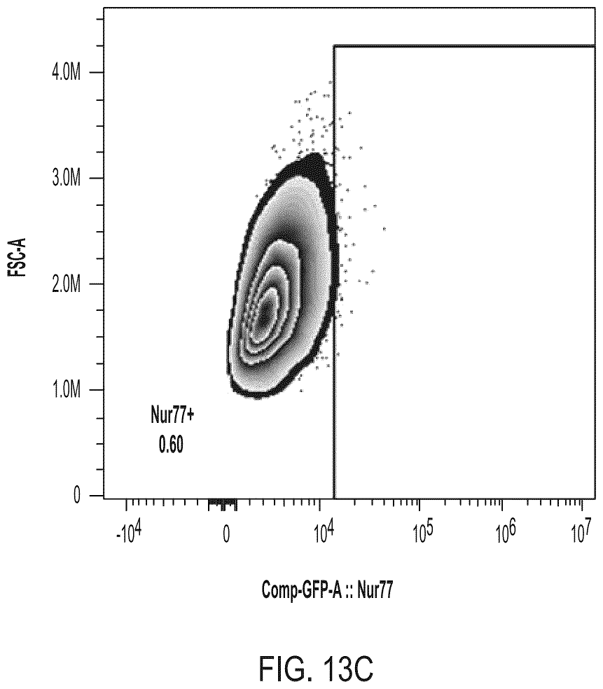

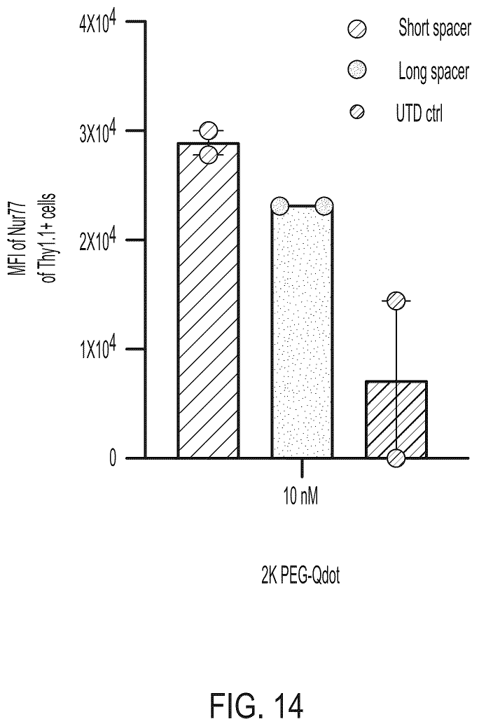

Non-limiting exemplary CAR regions and sequences are provided in Table 1, including amino acid and nucleic acid sequences for various CAR constructs shown in FIGS. 6, 10A, and 11A.

TABLE 1

| | | | |
|---|---|---|---|
| | | | SEQ ID |
| CAR regions | Sequence | UniProt Id | NO |

CD19 CAR:

| | | | |
|---|---|---|---|
| GMCSFR | MLLLVTSLLLCELPHPAFLLIP | | 1 |
| Signal Peptide | | | |
| FMC63 VH | EVKLQESGPGLVAPSQSLSVTCTVSGVSLPDY | | 2 |
| | GVSWIRQPPRKGLEWLGVIWGSETTYYNSAL | | |
| | KSRLTIIKDNSKSQVFLKMNSLQTDDTAIYYC | | |
| | AKHYYYGGSYAMDYWGQGTSVTVSS | | |
| Whitlow Linker | GSTSGSGKPGSGEGSTKG | | 3 |
| FMC63 VL | DIQMTQTTSSLSASLGDRVTISCRASQDISKY | | 4 |
| | LNWYQQKPDGTVKLLIYHTSRLHSGVPSRFS | | |
| | GSGSGTDYSLTISNLEQEDIATYFCQQGNTLP | | |
| | YTFGGGTKLEIT | | |
| CD28 | IEVMYPPPYLDNEKSNGTIIHVKGKHLCPSPL | P10747-1 | 5 |
| (AA 114-220) | FPGPSKPFWVLVVVGGVLACYSLLVTVAFIIF | | |
| | WVRSKRSRLLHSDYMNMTPRRPGPTRKHYQ | | |
| | PYAPPRDFAAYRS | | |
| CD3-zeta | RVKFSRSADAPAYQQGQNQLYNELNLGRRE | P20963-3 | 6 |
| isoform 3 | EYDVLDKRRGRDPEMGGKPRRKNPQEGLY | | |
| (AA 52-163) | NELQKDKMAEAYSEIGMKGERRRGKGHDG | | |
| | LYQGLSTATKDTYDALHMQALPPR | | |
| FMC63 scFV | EVKLQESGPGLVAPSQSLSVTCTVSGVSLPD | | 7 |
| | YGVSWIRQPPRKGLEWLGVIWGSETTYYNS | | |
| | ALKSRLTIIKDNSKSQVFLKMNSLQTDDTAIY | | |
| | YCAKHYYYGGSYAMDYWGQGTSVTVSSGS | | |
| | TSGSGKPGSGEGSTKGDIQMTQTTSSLSASL | | |
| | GDRVTISCRASQDISKYLNWYQQKPDGTVK | | |
| | LLIYHTSRLHSGVPSRFSGSGSGTDYSLTISNL | | |
| | EQEDIATYFCQQGNTLPYTFGGGTKLEIT | | |

CD22 CAR Antigen-binding Domains:

| | | |
|---|---|---|
| CD22_D04 | QVQLVESGGGLVQAGGSLRLSCAASGSEFT | 96 |
| | GYPMGWFRQAPGKEREFVAGSVGIGGSTNY | |
| | ADSVKGRFTISRDNAKNTVYLQMNSLKPED | |
| | TAVYYCAADKDYYKPYSRYRTVIRYETWG | |
| | QGTQVTVSS | |
| CD22_CNTY_ | EVQLLESGGGLVQPGGSLRLSCAASGLTSYS | 97 |
| VHH1_A01 | YAMGWYRQAPGKEREFVSAISSGGSAYYAD | |
| | SVKGRFTISRDNSKNTLYLQMNSLRAEDTAV | |
| | YYCAVGPYYGFRAVTEADYWGQGTQVTVS | |
| | S | |
| CD22_CNTY_ | EVQLLESGGGLVQPGGSLRLSCAASGFTSSS | 98 |
| VHH1_E04 | YVMGWYRQAPGKEREFVSSISTGGDAYYAD | |
| | SVKGRFTISRDNSKNTLYLQMNSLRAEDTAV | |
| | YYCAADVWYYHGGAYDYWGQGTQVTVSS | |

CD22 CAR w/o Antigen-Binding Domains:

| | | |
|---|---|---|
| IgG4(CH3)/ | IgG4 CH3 Hinge: | 102 |
| CD28/41BB/ | ESKYGPPCPPCPGQPREPQVYTLPPSQEEMT | |
| CD3z | KNQVSLTCLVKGFYPSDIAVEWESNGQPEN | |
| | NYKTTPPVLDSDGSFFLYSRLTVDKSRWQEG | |
| | NVFSCSVMHEALHNHYTQKSLSLSLGK | |
| | CD28 Transmembrane Domain: | 24 |
| | FWVLVVVGGVLACYSLLVTVAFIIFWV | |
| | 41BB Co-Stimulatory Domain: | 8 |
| | KRGRKKLLYIFKQPFMRPVQTTQEEDGCSCR | |
| | FPEEEEGGCEL | |
| | CD3z Signaling Domain: | 6 |
| | RVKFSRSADAPAYQQGQNQLYNELNLGRRE | |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |
| CD8/CD28/ | CD8 Hinge: | 21 |
| 41BB/CD3z | TTTPAPRPPTPAPTIASQPLSLRPEACRPAAG | |
| | GAVHTRGLDFACD | |
| | CD28 Transmembrane Domain: | 24 |
| | FWVLVVVGGVLACYSLLVTVAFIIFWV | |
| | 41BB Co-Stimulatory Domain: | 8 |
| | KRGRKKLLYIFKQPFMRPVQTTQEEDGCSCR | |
| | FPEEEEGGCEL | |
| | CD3z Signaling Domain: | 6 |
| | RVKFSRSADAPAYQQGQNQLYNELNLGRRE | |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |
| CD8/CD8/ | CD8 Hinge: | 21 |
| DAP10/CD3z | TTTPAPRPPTPAPTIASQPLSLRPEACRPAAG | |
| | GAVHTRGLDFACD | |
| | CD8 Transmembrane Domain: | 23 |
| | IYIWAPLAGTCGVLLLSLVIT | |
| | DAP10 Co-Stimulatory Domain: | 17 |
| | LCARPRRSPAQEDGKVYINMPGRG | |
| | CD3z Signaling Domain: | 6 |
| | RVKFSRSADAPAYQQGQNQLYNELNLGRRE | |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |

Signaling/Co-stimulatory Domains:

| | | | |
|---|---|---|---|
| 41BB | KRGRKKLLYIFKQPFMRPVQTTQEEDGCSCR | Q07011 | 8 |
| (AA 214-255) | FPEEEEGGCEL | | |
| IL2Rb | NCRNTGPWLKKVLKCNTPDPSKFFSQLSSEH | P14784 | 9 |
| (AA 266-551) | GGDVQKWLSSPFPSSSFSPGGLAPEISPLEVL | | |
| | ERDKVTQLLPLNTDAYLSLQELQGQDPTHL | | |
| | V | | |
| CD40 | KKVAKKPTNKAPHPKQEPQEINFPDDLPGSN | P25942 | 10 |
| (AA 216-277) | TAAPVQETLHGCQPVTQEDGKESRISVQERQ | | |
| OX40 | ALYLLRRDQRLPPDAHKPPGGGSFRTPIQEE | P43489 | 11 |
| (AA 236-277) | QADAHSTLAKI | | |
| CD80 | TYCFAPRCRERRRNERLRRESVRPV | P33681 | 12 |
| (AA 264-288) | | | |
| CD86 (AA269- | KWKKKKRPRNSYKCGTNTMEREESEQTKK | P42081 | 13 |
| 329) | REKIHIPERSDEAQRVFKSSKTSSCDKSDTCF | | |
| CD27 | QRRKYRSNKGESPVEPAEPCHYSCPREEEGS | P26842 | 14 |
| (AA 213-260) | TIPIQEDYRKPEPACSP | | |
| ICOS | CWLTKKKYSSSVHDPNGEYMFMRAVNTAK | Q9Y6W8 | 15 |
| (AA 162-199) | KSRLTDVTL | | |
| NKG2D | MGWIRGRRSR HSWEMSEFHN | P26718 | 16 |
| (AA 1-51) | YNLDLKKSDF STRWQKQRCP | | |
| | VVKSKCRENAS | | |
| DAP10 | LCARPRRSPAQEDGKVYINMPGRG | Q9UBK5 | 17 |
| (AA 70-93) | | | |
| DAP12 | YFLGRLVPRGRGAAEAATRKQRITETESPYQ | O54885 | 18 |
| (AA 62-113) | ELQGQRSDVYSDLNTQRPYYK | | |
| 2B4/CD244 | WRRKRKEKQSETSPKEFLTIYEDVKDLKTRR | Q9BZW8 | 19 |
| (AA 251-370) | NHEQEQTFPGGGSTIYSMIQSQSSAPTSQEPA | | |
| | YTLYSLIQPSRKSGSRKRNHSPSFNSTIYEVIG | | |
| | KSQPKAQNPARLSRKELENFDVYS | | |
| CD3-zeta | RVKFSRSADAPAYQQGQNQLYNELNLGRRE | P20963-3 | 6 |
| isoform 3 | EYDVLDKRRGRDPEMGGKPRRKNPQEGLY | | |
| (AA 52-163) | NELQKDKMAEAYSEIGMKGERRRGKGHDG | | |
| | LYQGLSTATKDTYDALHMQALPPR | | |
| CD28 | RSKRSRLLHSDYMNMTPRRPGPTRKHYQPY | P10747-1 | 20 |
| (AA 180-220) | APPRDFAAYRS | | |

Spacer/Hinge:

| | | | |
|---|---|---|---|
| CD8 | TTTPAPRPPTPAPTIASQPLSLRPEACRPAAG | P01732 | 21 |
| (AA 136-182) | GAVHTRGLDFACD | | |
| CD28 | IEVMYPPPYLDNEKSNGTIIHVKGKHLCPSPL | P10747-1 | 22 |
| (AA 114-151) | FPGPSKP | | |
| IgG4 CH3 | ESKYGPPCPPCPGQPREPQVYTLPPSQEEMT | | 102 |
| | KNQVSLTCLVKGFYPSDIA VEWESNGQPEN | | |
| | NYKTTPPVLDSDGSFFLYSRLTVDKSRWQEG | | |
| | NVFSCSVMHEALHNHYTQKSLSLSLGK | | |

Transmembrane:

| | | | |
|---|---|---|---|
| CD8 | IYIWAPLAGTCGVLLLSLVIT | P01732 | 23 |
| (AA 183-203) | | | |
| CD28 | FWVLVVVGGVLACYSLLVTVAFIIFWV | P10747-1 | 24 |
| (AA 153-179) | | | |

Linkers:

| | | |
|---|---|---|
| Whitlow Linker | GSTSGSGKPGSGEGSTKG | 3 |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 5 | GGGSGGGSGGGS | 28 |
| Linker 6 | GGGSGGGSGGGSGGGS | 29 |
| Linker 7 | GGGSGGGSGGGSGGGSGGGS | 30 |
| Linker 8 | GGGGSGGGGSGGGGSGGGGS | 31 |
| Linker 9 | GGGGSGGGGSGGGGSGGGGSGGGGS | 32 |
| Linker 10 | IRPRAIGGSKPRVA | 33 |
| Linker 11 | GKGGSGKGGSGKGGS | 34 |
| Linker 12 | GGKGSGGKGSGGKGS | 35 |
| Linker 13 | GGGKSGGGKSGGGKS | 36 |
| Linker 14 | GKGKSGKGKSGKGKS | 37 |
| Linker 15 | GGGKSGGKGSGKGGS | 38 |
| Linker 16 | GKPGSGKPGSGKPGS | 39 |
| Linker 17 | GKPGSGKPGSGKPGSGKPGS | 40 |
| Linker 18 | GKGKSGKGKSGKGKSGKGKS | 41 |
| Linker 19 | STAGDTHLGGEDFD | 42 |
| Linker 20 | GEGGSGEGGSGEGGS | 43 |
| Linker 21 | GGEGSGGEGSGGEGS | 44 |
| Linker 22 | GEGESGEGESGEGES | 45 |
| Linker 23 | GGGESGGEGSGEGGS | 46 |
| Linker 24 | GEGESGEGESGEGESGEGES | 47 |
| Linker 25 | GSTSGSGKPGSGEGSTKG | 48 |
| Linker 26 | PRGASKSGSASQTGSAPGS | 49 |
| Linker 27 | GTAAAGAGAAGGAAAGAAG | 50 |
| Linker 28 | GTSGSSGSGSGGSGSGGGG | 51 |
| Linker 29 | GKPGSGKPGSGKPGSGKPGS | 52 |
| Linker 30 | GSGS | 53 |
| Linker 31 | APAPAPAPAP | 54 |
| Linker 32 | APAPAPAPAPAPAPAPAPAP | 55 |
| Linker 33 | AEAAAKEAAAKEAAAAKEAAAAKEAAAAK | 56 |
| | AAA | |

| | | |
|---|---|---|
| | | SEQ ID |
| CAR regions | Sequence | NO |

Transmembrane:

| | | |
|---|---|---|
| IgK Signal | MARSPAQLLGLLLLWLSGARC | 103 |
| Peptide Variant | | |

Amino Acid Sequences of Mono-specific CARs Targeting CD19 or CD22:

| | | |
|---|---|---|
| FMC63_CD28_ | MLLLVTSLLLCELPHPAFLLIPDIQMTQTTSSLSASLGDRV | 104 |
| CD28_CD28_ | TISCRASQDISKYLNWYQQKPDGTVKLLIYHTSRLHSGVP | |
| CD3Z (P1209) | SRFSGSGSGTDYSLTISNLEQEDIATYFCQQGNTLPYTFGG | |
| | GTKLEITGSTSGSGKPGSGEGSTKGEVKLQESGPGLVAPS | |
| | QSLSVTCTVSGVSLPDYGVSWIRQPPRKGLEWLGVIWGS | |
| | ETTYYNSALKSRLTIIKDNSKSQVFLKMNSLQTDDTAIYY | |
| | CAKHYYYGGSYAMDYWGQGTSVTVSSIEVMYPPPYLDN | |
| | EKSNGTIIHVKGKHLCPSPLFPGPSKPFWVLVVVGGVLAC | |
| | YSLLVTVAFIIFWVRSKRSRLLHSDYMNMTPRRPGPTRKH | |
| | YQPYAPPRDFAAYRSRVKFSRSADAPAYQQGQNQLYNE | |
| | LNLGRREEYDVLDKRRGRDPEMGGKPRRKNPQEGLYNE | |
| | LQKDKMAEAYSEIGMKGERRRGKGHDGLYQGLSTATKD | |
| | TYDALHMQALPPR | |
| A01_Ig4 | MARSPAQLLGLLLLWLSGARCEVOLLESGGGLVQPGGSL | 105 |
| CH3_CD28_ | RLSCAASGLTSYSYAMGWYRQAPGKEREFVSAISSGGSA | |
| 41BB_CD3Z | YYADSVKGRFTISRDNSKNTLYLQMNSLRAEDTAVYYC | |
| (P1362) | AVGPYYGFRAVTEADYWGQGTQVTVSSESKYGPPCPPCP | |
| | GQPREPQVYTLPPSQEEMTKNQVSLTCLVKGFYPSDIAVE | |
| | WESNGQPENNYKTTPPVLDSDGSFFLYSRLTVDKSRWQE | |
| | GNVFSCSVMHEALHNHYTQKSLSLSLGKFWVLVVVGGV | |
| | LACYSLLVTVAFIIFWVKRGRKKLLYIFKQPFMRPVQTTQ | |
| | EEDGCSCRFPEEEEGGCELRVKFSRSADAPAYQQGQNQL | |
| | YNELNLGRREEYDVLDKRRGRDPEMGGKPRRKNPQEGL | |
| | YNELQKDKMAEAYSEIGMKGERRRGKGHDGLYQGLSTA | |
| | TKDTYDALHMQALPPR | |
| A01- | MARSPAQLLGLLLLWLSGARCEVQLLESGGGLVQPGGSL | 106 |
| A01_CD28_ | RLSCAASGLTSYSYAMGWYRQAPGKEREFVSAISSGGSA | |
| CD3Z (P1631) | YYADSVKGRFTISRDNSKNTLYLQMNSLRAEDTAVYYC | |
| | AVGPYYGFRAVTEADYWGQGTQVTVSSGSTSGSGKPGS | |
| | GEGSTKGEVQLLESGGGLVQPGGSLRLSCAASGLTSYSY | |
| | AMGWYRQAPGKEREFVSAISSGGSAYYADSVKGRFTISR | |
| | DNSKNTLYLQMNSLRAEDTAVYYCAVGPYYGFRAVTEA | |
| | DYWGQGTQVTVSSIEVMYPPPYLDNEKSNGTIIHVKGKH | |
| | LCPSPLFPGPSKPFWVLVVVGGVLACYSLLVTVAFIIFWV | |
| | RSKRSRLLHSDYMNMTPRRPGPTRKHYQPYAPPRDFAAY | |
| | RSRVKFSRSADAPAYQQGQNQLYNELNLGRREEYDVLD | |
| | KRRGRDPEMGGKPRRKNPQEGLYNELQKDKMAEAYSEI | |
| | GMKGERRRGKGHDGLYQGLSTATKDTYDALHMQALPP | |
| | R | |
| D04AMD11_ | MARSPAQLLGLLLLWLSGARCQVQLVESGGGLVQAGGS | 107 |
| CD8_CD28_ | LRLSCAASGSEFTGYPMGWFRQAPGKEREFVAGSVGIGG | |
| 41BB_CD3Z | STNYADSVKGRFTISRDNAKNTVYLQMNSLKPEDTAVYY | |
| (P1729) | CAADKDYYKPYSRYRTAIRYDTWGQGTQVTVSSTTTPAP | |
| | RPPTPAPTIASQPLSLRPEACRPAAGGAVHTRGLDFACDF | |
| | WVLVVVGGVLACYSLLVTVAFIIFWVKRGRKKLLYIFKQ | |
| | PFMRPVQTTQEEDGCSCRFPEEEEGGCELRVKFSRSADAP | |
| | AYQQGQNQLYNELNLGRREEYDVLDKRRGRDPEMGGK | |
| | PRRKNPQEGLYNELQKDKMAEAYSEIGMKGERRRGKGH | |
| | DGLYQGLSTATKDTYDALHMQALPPR | |
| E04- | MARSPAQLLGLLLLWLSGARCEVQLLESGGGLVQPGGSL | 108 |
| E04_CD28_ | RLSCAASGFTSSSYVMGWYRQAPGKEREFVSSISTGGDA | |
| CD28_CD28_ | YYADSVKGRFTISRDNSKNTLYLQMNSLRAEDTAVYYC | |
| CD3Z (P1633) | AADVWYYHGGAYDYWGQGTQVTVSSGSTSGSGKPGSG | |
| | EGSTKGEVQLLESGGGLVQPGGSLRLSCAASGFTSSSYV | |
| | MGWYRQAPGKEREFVSSISTGGDAYYADSVKGRFTISRD | |
| | NSKNTLYLQMNSLRAEDTAVYYCAADVWYYHGGAYDY | |
| | WGQGTQVTVSSIEVMYPPPYLDNEKSNGTIIHVKGKHLC | |
| | PSPLFPGPSKPFWVLVVVGGVLACYSLLVTVAFIIFWVRS | |
| | KRSRLLHSDYMNMTPRRPGPTRKHYQPYAPPRDFAAYRS | |
| | RVKFSRSADAPAYQQGQNQLYNELNLGRREEYDVLDKR | |
| | RGRDPEMGGKPRRKNPQEGLYNELQKDKMAEAYSEIGM | |
| | KGERRRGKGHDGLYQGLSTATKDTYDALHMQALPPR | |
| D04AMD11- | MARSPAQLLGLLLLWLSGARCQVQLVESGGGLVQAGGS | 109 |
| E04_CD28_ | LRLSCAASGSEFTGYPMGWFRQAPGKEREFVAGSVGIGG | |
| CD28_CD28_ | STNYADSVKGRFTISRDNAKNTVYLQMNSLKPEDTAVYY | |
| CD3Z (P1702) | CAADKDYYKPYSRYRTAIRYDTWGQGTQVTVSSGSTSGS | |
| | GKPGSGEGSTKGEVQLLESGGGLVQPGGSLRLSCAASGF | |
| | TSSSYVMGWYRQAPGKEREFVSSISTGGDAYYADSVKGR | |
| | FTISRDNSKNTLYLQMNSLRAEDTAVYYCAADVWYYHG | |
| | GAYDYWGQGTQVTVSSIEVMYPPPYLDNEKSNGTIIHVK | |
| | GKHLCPSPLFPGPSKPFWVLVVVGGVLACYSLLVTVAFII | |
| | FWVRSKRSRLLHSDYMNMTPRRPGPTRKHYQPYAPPRDF | |
| | AAYRSRVKFSRSADAPAYQQGQNQLYNELNLGRREEYD | |
| | VLDKRRGRDPEMGGKPRRKNPQEGLYNELQKDKMAEA | |
| | YSEIGMKGERRRGKGHDGLYQGLSTATKDTYDALHMQA | |
| | LPPR | |
| E04_Ig4_ | MARSPAQLLGLLLLWLSGARCEVQLLESGGGLVQPGGSL | 110 |
| CD28_41BB_ | RLSCAASGFTSSSYVMGWYRQAPGKEREFVSSISTGGDA | |
| CD3Z (P1356) | YYADSVKGRFTISRDNSKNTLYLQMNSLRAEDTAVYYC | |
| | AADVWYYHGGAYDYWGQGTQVTVSSESKYGPPCPPCP | |
| | GKFWVLVVVGGVLACYSLLVTVAFIIFWVKRGRKKLLYI | |
| | FKQPFMRPVQTTQEEDGCSCRFPEEEEGGCELRVKFSRSA | |
| | DAPAYQQGQNQLYNELNLGRREEYDVLDKRRGRDPEM | |
| | GGKPRRKNPQEGLYNELQKDKMAEAYSEIGMKGERRRG | |
| | KGHDGLYQGLSTATKDTYDALHMQALPPR | |
| D04AMD11- | MARSPAQLLGLLLLWLSGARCQVQLVESGGGLVQAGGS | 111 |
| A01_CD8_CD28_ | LRLSCAASGSEFTGYPMGWFRQAPGKEREFVAGSVGIGG | |
| 41BB_CD3Z | STNYADSVKGRFTISRDNAKNTVYLQMNSLKPEDTAVYY | |
| (P1734) | CAADKDYYKPYSRYRTAIRYDTWGQGTQVTVSSGSTSGS | |
| | GKPGSGEGSTKGEVQLLESGGGLVQPGGSLRLSCAASGL | |
| | TSYSYAMGWYRQAPGKEREFVSAISSGGSAYYADSVKG | |
| | RFTISRDNSKNTLYLQMNSLRAEDTAVYYCAVGPYYGFR | |
| | AVTEADYWGQGTQVTVSSTTTPAPRPPTPAPTIASQPLSL | |
| | RPEACRPAAGGAVHTRGLDFACDFWVLVVVGGVLACYS | |
| | LLVTVAFIIFWVKRGRKKLLYIFKQPFMRPVQTTQEEDGC | |
| | SCRFPEEEEGGCELRVKFSRSADAPAYQQGQNQLYNELN | |
| | LGRREEYDVLDKRRGRDPEMGGKPRRKNPQEGLYNELQ | |
| | KDKMAEAYSEIGMKGERRRGKGHDGLYQGLSTATKDTY | |
| | DALHMQALPPR | |

Polynucleotide Sequences of Mono-specific CARs Targeting CD19 or CD22:

| | | |
|---|---|---|
| FMC63_CD28_ | ATGCTGCTGCTGGTTACATCTCTGCTGCTGTGCGAGCT | 112 |
| CD28_CD28_ | GCCCCATCCTGCCTTTCTGCTGATCCCCGACATCCAGA | |
| CD3Z (P1209) | TGACCCAGACCACAAGCAGCCTGTCTGCCAGCCTGGGC | |
| | GATAGAGTGACCATCAGCTGTAGAGCCAGCCAGGACA | |
| | TCAGCAAGTACCTGAACTGGTATCAGCAAAAGCCCGA | |
| | CGGCACCGTGAAGCTGCTGATCTACCACACCAGCAGA | |
| | CTGCACAGCGGCGTGCCAAGCAGATTTTCTGGCAGCGG | |
| | CTCTGGCACCGACTACAGCCTGACAATCAGCAACCTGG | |
| | AACAAGAGGATATCGCTACCTACTTCTGCCAGCAAGGC | |
| | AACACCCTGCCTTACACCTTTGGCGGAGGCACCAAGCT | |
| | GGAAATCACCGGCTCTACAAGCGGCAGCGGCAAACCT | |
| | GGATCTGGCGAGGGATCTACCAAGGGCGAAGTGAAAC | |
| | TGCAAGAGTCTGGCCCTGGACTGGTGGCCCCATCTCAG | |
| | TCTCTGAGCGTGACCTGTACAGTCAGCGGAGTGTCCCT | |
| | GCCTGATTACGGCGTGTCCTGGATCAGACAGCCTCCTC | |
| | GGAAAGGCCTGGAATGGCTGGGAGTGATCTGGGGCAG | |
| | CGAGACAACCTACTACAACAGCGCCCTGAAGTCCCGG | |
| | CTGACCATCATCAAGGACAACTCCAAGAGCCAGGTGTT | |
| | CCTGAAGATGAACAGCCTGCAGACCGACGACACCGCC | |
| | ATCTACTATTGCGCCAAGCACTACTACTACGGCGGCAG | |
| | CTACGCCATGGATTATTGGGGCCAGGGCACCAGCGTG | |
| | ACCGTGTCTAGCATCGAAGTGATGTACCCTCCACCTTA | |
| | CCTGGACAACGAGAAGTCCAACGGCACCATCATCCAC | |
| | GTGAAGGGCAAGCACCTGTGTCCTTCTCCACTGTTCCC | |
| | CGGACCTAGCAAGCCTTTCTGGGTGCTCGTTGTTGTTG | |
| | GCGGCGTGCTGGCCTGTTATAGCCTGCTTGTGACCGTG | |
| | GCCTTCATCATCTTTTGGGTCCGAAGCAAGCGGAGCCG | |
| | GCTGCTGCACTCCGACTACATGAACATGACCCCTAGAC | |
| | GGCCCGGACCAACCAGAAAGCACTACCAGCCTTACGC | |
| | TCCTCCTAGAGACTTCGCCGCCTACCGGTCCAGAGTGA | |
| | AGTTCAGCAGATCCGCCGATGCTCCCGCCTATCAGCAG | |
| | GGCCAAAACCAGCTGTACAACGAGCTGAACCTGGGGA | |
| | GAAGAGAAGAGTACGACGTGCTGGACAAGCGGAGAG | |
| | GCAGAGATCCTGAAATGGGCGGCAAGCCCAGACGGAA | |
| | GAATCCTCAAGAGGGCCTGTATAATGAGCTGCAGAAA | |
| | GACAAGATGGCCGAGGCCTACAGCGAGATCGGAATGA | |
| | AGGGCGAGCGCAGAAGAGGCAAGGGACACGATGGAC | |
| | TGTACCAGGGCCTGAGCACCGCCACCAAGGATACCTAT | |
| | GATGCCCTGCACATGCAGGCCCTGCCTCCAAGA | |
| A01_Ig4 | ATGGCCAGATCTCCTGCTCAACTGCTGGGACTGCTGCT | 113 |
| CH3_CD28_ | GCTGTGGCTTAGCGGAGCCAGATGCGAGGTACAACTTT | |
| 41BB_CD3Z | TGGAGTCAGGCGGTGGACTGGTACAACCGGGTGGTTC | |
| (P1362) | ATTGCGTTTGAGCTGCGCTGCCTCTGGTTTGACCTCTTA | |
| | TTCCTACGCGATGGGCTGGTATCGCCAAGCGCCGGGCA | |
| | AAGAACGCGAGTTTGTCAGCGCAATCAGCTCGGGTGG | |
| | TAGCGCGTACTACGCGGACTCGGTAAAAGGCCGTTTTA | |
| | CGATCAGTCGTGATAATTCCAAGAATACCTTGTACCTG | |
| | CAAATGAATAGCCTTCGCGCAGAAGACACAGCGGTGT | |
| | ATTATTGTGCCGTTGGACCGTACTACGGATTTAGAGCG | |
| | GTTACCGAAGCAGATTATTGGGGCCAGGGTACCCAGG | |
| | TGACGGTCTCGAGCGAGTCCAAATACGGTCCGCCATGC | |
| | CCACCATGCCCAGGGCAGCCCCGAGAGCCACAGGTGT | |
| | ACACCCTGCCCCCATCCCAAGAGGAGATGACCAAGAA | |
| | CCAAGTCAGCCTGACCTGCCTGGTCAAAGGCTTCTACC | |
| | CCAGCGACATCGCCGTGGAGTGGGAGAGCAATGGGCA | |
| | GCCGGAGAACAACTACAAGACCACGCCTCCCGTGCTG | |
| | GACTCCGACGGCTCCTTCTTCCTCTACTCCCGGCTCACC | |
| | GTGGACAAGAGCAGGTGGCAGGAGGGGAATGTGTTCT | |
| | CATGCTCCGTGATGCATGAGGCTCTGCACAACCACTAC | |
| | ACACAGAAGAGCCTCTCCCTGTCTCTGGGAAAGTTTTG | |
| | GGTGCTGGTGGTGGTTGGTGGAGTCCTGGCTTGCTATT | |
| | CCTTGCTAGTAACAGTGGCCTTTATTATTTTCTGGGTGA | |
| | AACGCGGCCGCAAGAAACTCCTGTATATATTCAAACA | |
| | ACCATTTATGAGGCCAGTACAAACTACTCAAGAGGAA | |
| | GATGGCTGTAGCTGCCGATTTCCAGAAGAAGAAGAAG | |
| | GAGGATGTGAGCTCAGAGTGAAGTTCAGCAGGAGCGC | |
| | AGACGCCCCCGCGTACCAGCAGGGCCAGAACCAGCTC | |
| | TATAACGAACTCAATCTAGGACGAAGAGAGGAGTACG | |
| | ATGTTTTGGACAAGCGGCGTGGCCGGGACCCTGAGAT | |
| | GGGGGGAAAGCCGAGAAGGAAGAACCCTCAGGAAGG | |
| | CCTGTACAATGAACTGCAGAAAGATAAGATGGCGGAG | |
| | GCCTACAGTGAGATTGGGATGAAAGGCGAGCGCCGGA | |
| | GGGGCAAGGGGCACGATGGCCTTTACCAGGGGCTCAG | |
| | TACAGCCACCAAGGACACCTACGACGCCCTTCACATGC | |
| | AGGCCCTGCCCCCTCGC | |
| A01- | ATGGCCAGATCTCCTGCTCAACTGCTGGGACTGCTGCT | 114 |
| A01_CD28_ | GCTGTGGCTTAGCGGAGCCAGATGCGAGGTACAACTTT | |
| CD3Z (P1631) | TGGAGTCAGGCGGTGGACTGGTACAACCGGGTGGTTC | |
| | ATTGCGTTTGAGCTGCGCTGCCTCTGGTTTGACCTCTTA | |
| | TTCCTACGCGATGGGCTGGTATCGCCAAGCGCCGGGCA | |
| | AAGAACGCGAGTTTGTCAGCGCAATCAGCTCGGGTGG | |
| | TAGCGCGTACTACGCGGACTCGGTAAAAGGCCGTTTTA | |
| | CGATCAGTCGTGATAATTCCAAGAATACCTTGTACCTG | |
| | CAAATGAATAGCCTTCGCGCAGAAGACACAGCGGTGT | |
| | ATTATTGTGCCGTTGGACCGTACTACGGATTTAGAGCG | |
| | GTTACCGAAGCAGATTATTGGGGCCAGGGTACCCAGG | |
| | TGACGGTCTCGAGCGGCTCTACAAGCGGCAGCGGCAA | |
| | ACCTGGATCTGGCGAGGGATCTACCAAGGGCGAGGTA | |
| | CAACTTTTGGAGTCAGGCGGTGGACTGGTACAACCGG | |
| | GTGGTTCATTGCGTTTGAGCTGCGCTGCCTCTGGTTTGA | |
| | CCTCTTATTCCTACGCGATGGGCTGGTATCGCCAAGCG | |
| | CCGGGCAAAGAACGCGAGTTTGTCAGCGCAATCAGCT | |
| | CGGGTGGTAGCGCGTACTACGCGGACTCGGTAAAAGG | |
| | CCGTTTTACGATCAGTCGTGATAATTCCAAGAATACCT | |
| | TGTACCTGCAAATGAATAGCCTTCGCGCAGAAGACAC | |
| | AGCGGTGTATTATTGTGCCGTTGGACCGTACTACGGAT | |
| | TTAGAGCGGTTACCGAAGCAGATTATTGGGGCCAGGG | |
| | TACCCAGGTGACGGTCTCGAGCATCGAAGTGATGTACC | |
| | CTCCACCTTACCTGGACAACGAGAAGTCCAACGGCACC | |
| | ATCATCCACGTGAAGGGCAAGCACCTGTGTCCTTCTCC | |
| | ACTGTTCCCCGGACCTAGCAAGCCTTTCTGGGTGCTCG | |
| | TTGTTGTTGGCGGCGTGCTGGCCTGTTATAGCCTGCTTG | |
| | TGACCGTGGCCTTCATCATCTTTTGGGTCCGAAGCAAG | |
| | CGGAGCCGGCTGCTGCACTCCGACTACATGAACATGAC | |
| | CCCTAGACGGCCCGGACCAACCAGAAAGCACTACCAG | |
| | CCTTACGCTCCTCCTAGAGACTTCGCCGCCTACCGGTC | |
| | CAGAGTGAAGTTCAGCAGATCCGCCGATGCTCCCGCCT | |
| | ATCAGCAGGGCCAAAACCAGCTGTACAACGAGCTGAA | |
| | CCTGGGGAGAAGAGAAGAGTACGACGTGCTGGACAAG | |
| | CGGAGAGGCAGAGATCCTGAAATGGGCGGCAAGCCCA | |
| | GACGGAAGAATCCTCAAGAGGGCCTGTATAATGAGCT | |
| | GCAGAAAGACAAGATGGCCGAGGCCTACAGCGAGATC | |
| | GGAATGAAGGGCGAGCGCAGAAGAGGCAAGGGACAC | |
| | GATGGACTGTACCAGGGCCTGAGCACCGCCACCAAGG | |
| | ATACCTATGATGCCCTGCACATGCAGGCCCTGCCTCCA | |
| | AGATAA | |
| D04AMD11_ | ATGGCCAGATCTCCTGCTCAACTGCTGGGACTGCTGCT | 115 |
| CD8_CD28_ | GCTGTGGCTTAGCGGAGCCAGATGCCAGGTGCAGCTG | |
| 41BB_CD3Z | GTTGAGTCTGGGGGAGGCCTTGTCCAGGCTGGGGGGTC | |
| (P1729) | CCTGAGACTCTCCTGTGCAGCGTCTGGAAGCGAATTCA | |
| | CCGGTTATCCCATGGGCTGGTTTCGCCAGGCTCCAGGC | |
| | AAGGAAAGGGAGTTTGTCGCTGGCTCCGTAGGTATCG | |
| | GTGGTAGTACAAACTATGCAGACTCCGTGAAGGGCCG | |
| | ATTCACCATCTCCAGAGACAATGCGAAGAACACGGTCT | |
| | ATCTGCAAATGAACAGCCTGAAGCCAGAGGACACGGC | |
| | TGTGTATTACTGTGCGGCCGACAAAGACTACTACAAAC | |
| | CTTATAGTCGATATAGGACCGCTATCAGGTACGATACC | |
| | TGGGGCCAAGGGACCCAGGTCACCGTCTCGAGCACAA | |
| | CAACTCCAGCCCCAAGACCACCTACGCCTGCACCTACT | |
| | ATCGCATCTCAACCACTGTCCCTGCGCCCTGAGGCATG | |
| | CCGACCAGCAGCCGGTGGCGCGGTGCATACCCGCGGA | |
| | CTGGACTTTGCCTGCGATTTTTGGGTGCTGGTGGTGGTT | |
| | GGTGGAGTCCTGGCTTGCTATAGCTTGCTAGTAACAGT | |
| | GGCCTTTATTATTTTCTGGGTGAAACGGGGCAGAAAGA | |
| | AACTCCTGTATATATTCAAACAACCATTTATGCGACCA | |
| | GTACAAACTACTCAAGAGGAAGATGGCTGTAGCTGCC | |
| | GATTTCCAGAAGAAGAAGAAGGAGGATGTGAACTGAG | |
| | AGTGAAGTTCAGCAGGAGCGCAGACGCCCCCGCGTAC | |
| | CAGCAGGGCCAGAACCAGCTCTATAACGAGCTCAATC | |
| | TAGGACGAAGAGAGGAGTACGATGTTTTGGACAAGCG | |
| | ACGTGGCCGGGACCCTGAGATGGGGGGAAAGCCGAGA | |
| | AGGAAGAACCCTCAGGAAGGCCTGTACAATGAACTGC | |
| | AGAAAGATAAGATGGCGGAGGCCTACAGTGAGATTGG | |
| | GATGAAAGGCGAGCGCCGGAGGGGCAAGGGGCACGA | |
| | TGGCCTTTACCAGGGACTCAGTACAGCCACCAAGGAC | |
| | ACCTACGACGCCCTTCACATGCAGGCCCTGCCCCCTCG | |
| | C | |
| E04- | ATGGCCAGATCTCCTGCTCAACTGCTGGGACTGCTGCT | |
| E04_CD28_ | GCTGTGGCTTAGCGGAGCCAGATGCGAGGTGCAGCTG | |
| CD28_CD28_ | TTGGAGAGCGGCGGGGGACTTGTTCAACCCGGAGGCT | |
| CD3Z | CTCTTAGGTTATCTTGCGCAGCTAGTGGATTTACGAGC | |
| (P1633) | TCCAGTTACGTGATGGGATGGTATCGACAGGCTCCTGG | |
| | AAAAGAAAGAGAGTTCGTGAGCTCTATTAGCACCGGC | |
| | GGCGATGCGTATTACGCAGATTCAGTGAAAGGCCGATT | |
| | CACCATTTCCAGGGATAACTCCAAAAACACTCTCTACC | |
| | TGCAAATGAACAGCCTGAGAGCCGAAGACACCGCTGT | |
| | TTATTATTGCGCCGCCGACGTTTGGTATTACCACGGAG | |
| | GCGCTTATGATTATTGGGGCCAGGGGACTCAGGTGACG | |
| | GTCTCATCTGGCTCTACAAGCGGCAGCGGCAAACCTGG | |
| | ATCTGGCGAGGGATCTACCAAGGGCGAGGTACAACTT | |
| | TTGGAGTCAGGCGGTGGACTGGTACAACCGGGTGGTTC | |
| | ATTGCGTTTGAGCTGCGCTGCCTCTGGTTTTACCAGCTC | |
| | CTCCTACGTGATGGGCTGGTATCGCCAAGCGCCGGGCA | 116 |
| | AAGAACGCGAGTTTGTCAGCTCGATCAGCACCGGTGGT | |
| | GATGCCTACTACGCGGACTCGGTAAAAGGCCGTTTTAC | |
| | GATCAGTCGTGATAATTCCAAGAATACCTTGTACCTGC | |
| | AAATGAATAGCCTTCGCGCAGAAGACACAGCGGTGTA | |
| | TTATTGTGCCGCTGACGTTTGGTACTACCACGGCGGCG | |
| | CGTACGATTATTGGGGCCAGGGTACCCAGGTGACGGTC | |
| | TCGAGCATCGAAGTGATGTACCCTCCACCTTACCTGGA | |
| | CAACGAGAAGTCCAACGGCACCATCATCCACGTGAAG | |
| | GGCAAGCACCTGTGTCCTTCTCCACTGTTCCCCGGACC | |
| | TAGCAAGCCTTTCTGGGTGCTCGTTGTTGTTGGCGGCG | |
| | TGCTGGCCTGTTATAGCCTGCTTGTGACCGTGGCCTTC | |
| | ATCATCTTTTGGGTCCGAAGCAAGCGGAGCCGGCTGCT | |
| | GCACTCCGACTACATGAACATGACCCCTAGACGGCCCG | |
| | GACCAACCAGAAAGCACTACCAGCCTTACGCTCCTCCT | |
| | AGAGACTTCGCCGCCTACCGGTCCAGAGTGAAGTTCAG | |
| | CAGATCCGCCGATGCTCCCGCCTATCAGCAGGGCCAAA | |
| | ACCAGCTGTACAACGAGCTGAACCTGGGGAGAAGAGA | |
| | AGAGTACGACGTGCTGGACAAGCGGAGAGGCAGAGAT | |
| | CCTGAAATGGGCGGCAAGCCCAGACGGAAGAATCCTC | |
| | AAGAGGGCCTGTATAATGAGCTGCAGAAAGACAAGAT | |
| | GGCCGAGGCCTACAGCGAGATCGGAATGAAGGGCGAG | |
| | CGCAGAAGAGGCAAGGGACACGATGGACTGTACCAGG | |
| | GCCTGAGCACCGCCACCAAGGATACCTATGATGCCCTG | |
| | CACATGCAGGCCCTGCCTCCAAGA | |
| D04AMD11- | ATGGCCAGATCTCCTGCTCAACTGCTGGGACTGCTGCT | |
| E04_CD28_ | GCTGTGGCTTAGCGGAGCCAGATGCCAGGTGCAGCTG | |
| CD28_CD28_ | GTTGAGTCTGGGGGAGGCCTTGTCCAGGCTGGGGGGTC | |
| CD3Z | CCTGAGACTCTCCTGTGCAGCGTCTGGAAGCGAATTCA | |
| (P1702) | CCGGTTATCCCATGGGCTGGTTTCGCCAGGCTCCAGGC | |
| | AAGGAAAGGGAGTTTGTCGCTGGCTCCGTAGGTATCG | |
| | GTGGTAGTACAAACTATGCAGACTCCGTGAAGGGCCG | |
| | ATTCACCATCTCCAGAGACAATGCGAAGAACACGGTCT | |
| | ATCTGCAAATGAACAGCCTGAAGCCAGAGGACACGGC | |
| | TGTGTATTACTGTGCGGCCGACAAAGACTACTACAAAC | |
| | CTTATAGTCGATATAGGACCGCTATCAGGTACGATACC | |
| | TGGGGCCAAGGGACCCAGGTCACCGTCTCGAGCGGGT | |
| | CTACCTCAGGGTCAGGGAAACCCGGAAGCGGCGAAGG | |
| | GTCTACAAAAGGTGAGGTACAACTTTTGGAGTCAGGC | |
| | GGTGGACTGGTACAACCGGGTGGTTCATTGCGTTTGAG | |
| | CTGCGCTGCCTCTGGTTTTACCAGCTCCTCCTACGTGAT | 117 |
| | GGGCTGGTATCGCCAAGCGCCGGGCAAAGAACGCGAG | |
| | TTTGTCAGCTCGATCAGCACCGGTGGTGATGCCTACTA | |
| | CGCGGACTCGGTAAAAGGCCGTTTTACGATCAGTCGTG | |
| | ATAATTCCAAGAATACCTTGTACCTGCAAATGAATAGC | |
| | CTTCGCGCAGAAGACACAGCGGTGTATTATTGTGCCGC | |
| | TGACGTTTGGTACTACCACGGCGGCGCGTACGATTATT | |
| | GGGGCCAGGGTACCCAGGTGACGGTCTCGAGCATCGA | |
| | AGTGATGTACCCTCCACCTTACCTGGACAACGAGAAGT | |
| | CCAACGGCACCATCATCCACGTGAAGGGCAAGCACCT | |
| | GTGTCCTTCTCCACTGTTCCCCGGACCTAGCAAGCCTTT | |
| | CTGGGTGCTCGTTGTTGTTGGCGGCGTGCTGGCCTGTT | |
| | ATAGCCTGCTTGTGACCGTGGCCTTCATCATCTTTTGGG | |
| | TCCGAAGCAAGCGGAGCCGGCTGCTGCACTCCGACTA | |
| | CATGAACATGACCCCTAGACGGCCCGGACCAACCAGA | |
| | AAGCACTACCAGCCTTACGCTCCTCCTAGAGACTTCGC | |
| | CGCCTACCGGTCCAGAGTGAAGTTCAGCAGGAGCGCA | |
| | GACGCCCCCGCGTACCAGCAGGGCCAGAACCAGCTCT | |
| | ATAACGAGCTCAATCTAGGACGAAGAGAGGAGTACGA | |
| | TGTTTTGGACAAGCGACGTGGCCGGGACCCTGAGATG | |
| | GGGGGAAAGCCGAGAAGGAAGAACCCTCAGGAAGGC | |
| | CTGTACAATGAACTGCAGAAAGATAAGATGGCGGAGG | |
| | CCTACAGTGAGATTGGGATGAAAGGCGAGCGCCGGAG | |
| | GGGCAAGGGGCACGATGGCCTTTACCAGGGACTCAGT | |
| | ACAGCCACCAAGGACACCTACGACGCCCTTCACATGC | |
| | AGGCCCTGCCCCCTCGC | |
| E04_Ig4_ | ATGGCCAGATCTCCTGCTCAACTGCTGGGACTGCTGCT | 118 |
| CD28_41BB_ | GCTGTGGCTTAGCGGAGCCAGATGCGAGGTACAACTTT | |
| CD3Z(P1356) | TGGAGTCAGGCGGTGGACTGGTACAACCGGGTGGTTC | |
| | ATTGCGTTTGAGCTGCGCTGCCTCTGGTTTTACCAGCTC | |
| | CTCCTACGTGATGGGCTGGTATCGCCAAGCGCCGGGCA | |
| | AAGAACGCGAGTTTGTCAGCTCGATCAGCACCGGTGGT | |
| | GATGCCTACTACGCGGACTCGGTAAAAGGCCGTTTTAC | |
| | GATCAGTCGTGATAATTCCAAGAATACCTTGTACCTGC | |
| | AAATGAATAGCCTTCGCGCAGAAGACACAGCGGTGTA | |
| | TTATTGTGCCGCTGACGTTTGGTACTACCACGGCGGCG | |
| | CGTACGATTATTGGGGCCAGGGTACCCAGGTGACGGTC | |
| | TCGAGCGAGTCCAAATACGGTCCGCCATGCCCACCATG | |
| | CCCAGGAAAGTTTTGGGTGCTGGTGGTGGTTGGTGGAG | |
| | TCCTGGCTTGCTATTCCTTGCTAGTAACAGTGGCCTTTA | |
| | TTATTTTCTGGGTGAAACGCGGCCGCAAGAAACTCCTG | |
| | TATATATTCAAACAACCATTTATGAGGCCAGTACAAAC | |
| | TACTCAAGAGGAAGATGGCTGTAGCTGCCGATTTCCAG | |
| | AAGAAGAAGAAGGAGGATGTGAGCTCAGAGTGAAGTT | |
| | CAGCAGGAGCGCAGACGCCCCCGCGTACCAGCAGGGC | |
| | CAGAACCAGCTCTATAACGAACTCAATCTAGGACGAA | |
| | GAGAGGAGTACGATGTTTTGGACAAGCGGCGTGGCCG | |
| | GGACCCTGAGATGGGGGGAAAGCCGAGAAGGAAGAA | |
| | CCCTCAGGAAGGCCTGTACAATGAACTGCAGAAAGAT | |
| | AAGATGGCGGAGGCCTACAGTGAGATTGGGATGAAAG | |
| | GCGAGCGCCGGAGGGGCAAGGGGCACGATGGCCTTTA | |
| | CCAGGGGCTCAGTACAGCCACCAAGGACACCTACGAC | |
| | GCCCTTCACATGCAGGCCCTGCCCCCTCGC | |
| D04AMD11- | ATGGCCAGATCTCCTGCTCAACTGCTGGGACTGCTGCT | 119 |
| A01_CD8_ | GCTGTGGCTTAGCGGAGCCAGATGCCAGGTGCAGCTG | |
| CD28_41BB_CD3Z | GTTGAGTCTGGGGGAGGCCTTGTCCAGGCTGGGGGGTC | |
| (P1734) | CCTGAGACTCTCCTGTGCAGCGTCTGGAAGCGAATTCA | |
| | CCGGTTATCCCATGGGCTGGTTTCGCCAGGCTCCAGGC | |
| | AAGGAAAGGGAGTTTGTCGCTGGCTCCGTAGGTATCG | |
| | GTGGTAGTACAAACTATGCAGACTCCGTGAAGGGCCG | |
| | ATTCACCATCTCCAGAGACAATGCGAAGAACACGGTCT | |
| | ATCTGCAAATGAACAGCCTGAAGCCAGAGGACACGGC | |
| | TGTGTATTACTGTGCGGCCGACAAAGACTACTACAAAC | |
| | CTTATAGTCGATATAGGACCGCTATCAGGTACGATACC | |
| | TGGGGCCAAGGGACCCAGGTCACCGTCTCGAGCGGGT | |
| | CTACCTCAGGGTCAGGGAAACCCGGAAGCGGCGAAGG | |
| | GTCTACAAAAGGTGAGGTACAACTTTTGGAGTCAGGC | |
| | GGTGGACTGGTACAACCGGGTGGTTCATTGCGTTTGAG | |
| | CTGCGCTGCCTCTGGTTTGACCTCTTATTCCTACGCGAT | |
| | GGGCTGGTATCGCCAAGCGCCGGGCAAAGAACGCGAG | |
| | TTTGTCAGCGCAATCAGCTCGGGTGGTAGCGCGTACTA | |
| | CGCGGACTCGGTAAAAGGCCGTTTTACGATCAGTCGTG | |
| | ATAATTCCAAGAATACCTTGTACCTGCAAATGAATAGC | |
| | CTTCGCGCAGAAGACACAGCGGTGTATTATTGTGCCGT | |
| | TGGACCGTACTACGGATTTAGAGCGGTTACCGAAGCA | |
| | GATTATTGGGGCCAGGGTACCCAGGTGACGGTCTCGA | |
| | GCACAACAACTCCAGCCCCAAGACCACCTACGCCTGC | |
| | ACCTACTATCGCATCTCAACCACTGTCCCTGCGCCCTG | |
| | AGGCATGCCGACCAGCAGCCGGTGGCGCGGTGCATAC | |
| | CCGCGGACTGGACTTTGCCTGCGATTTTTGGGTGCTGG | |
| | TGGTGGTTGGTGGAGTCCTGGCTTGCTATAGCTTGCTA | |
| | GTAACAGTGGCCTTTATTATTTTCTGGGTGAAACGGGG | |
| | CAGAAAGAAACTCCTGTATATATTCAAACAACCATTTA | |
| | TGCGACCAGTACAAACTACTCAAGAGGAAGATGGCTG | |
| | TAGCTGCCGATTTCCAGAAGAAGAAGAAGGAGGATGT | |
| | GAACTGAGAGTGAAGTTCAGCAGGAGCGCAGACGCCC | |
| | CCGCGTACCAGCAGGGCCAGAACCAGCTCTATAACGA | |
| | GCTCAATCTAGGACGAAGAGAGGAGTACGATGTTTTG | |
| | GACAAGCGACGTGGCCGGGACCCTGAGATGGGGGGAA | |
| | AGCCGAGAAGGAAGAACCCTCAGGAAGGCCTGTACAA | |
| | TGAACTGCAGAAAGATAAGATGGCGGAGGCCTACAGT | |
| | GAGATTGGGATGAAAGGCGAGCGCCGGAGGGGCAAG | |
| | GGGCACGATGGCCTTTACCAGGGACTCAGTACAGCCA | |
| | CCAAGGACACCTACGACGCCCTTCACATGCAGGCCCTG | |
| | CCCCCTCGC | |

Amino Acid Sequences of Bi-specific CARs Targeting CD19 and CD22:

| | | |
|---|---|---|
| FMC63LC_E04_ | MARSPAQLLGLLLLWLSGARCDIQMTQTTSSLSASLGDR | 120 |
| FMC63HC_ | VTISCRASQDISKYLNWYQQKPDGTVKLLIYHTSRLHSGV | |
| CD28_CD28_ | PSRFSGSGSGTDYSLTISNLEQEDIATYFCQQGNTLPYTFG | |
| CD28_CD3Z | GGTKLEITGSTSGSGKPGSGEGSTKGEVQLLESGGGLVQP | |
| (P1973) | GGSLRLSCAASGFTSSSYVMGWYRQAPGKEREFVSSISTG | |
| | GDAYYADSVKGRFTISRDNSKNTLYLQMNSLRAEDTAV | |
| | YYCAADVWYYHGGAYDYWGQGTQVTVSSGSTSGSGKP | |
| | GSGEGSTKGEVKLQESGPGLVAPSQSLSVTCTVSGVSLPD | |
| | YGVSWIRQPPRKGLEWLGVIWGSETTYYNSALKSRLTIIK | |
| | DNSKSQVFLKMNSLQTDDTAIYYCAKHYYYGGSYAMDY | |
| | WGQGTSVTVSSIEVMYPPPYLDNEKSNGTIIHVKGKHLCP | |
| | SPLFPGPSKPFWVLVVVGGVLACYSLLVTVAFIIFWVRSK | |
| | RSRLLHSDYMNMTPRRPGPTRKHYQPYAPPRDFAAYRSR | |
| | VKFSRSADAPAYQQGQNQLYNELNLGRREEYDVLDKRR | |
| | GRDPEMGGKPRRKNPQEGLYNELQKDKMAEAYSEIGMK | |
| | GERRRGKGHDGLYQGLSTATKDTYDALHMQALPPR | |
| FMC63LC_A01_ | MARSPAQLLGLLLLWLSGARCDIQMTQTTSSLSASLGDR | 121 |
| FMC63HC_ | VTISCRASQDISKYLNWYQQKPDGTVKLLIYHTSRLHSGV | |
| CD28_CD28_ | PSRFSGSGSGTDYSLTISNLEQEDIATYFCQQGNTLPYTFG | |
| CD28_CD3Z | GGTKLEITGSTSGSGKPGSGEGSTKGEVQLLESGGGLVQP | |
| (P1988) | GGSLRLSCAASGLTSYSYAMGWYRQAPGKEREFVSAISS | |
| | GGSAYYADSVKGRFTISRDNSKNTLYLQMNSLRAEDTAV | |
| | YYCAVGPYYGFRAVTEADYWGQGTQVTVSSGSTSGSGK | |
| | PGSGEGSTKGEVKLQESGPGLVAPSQSLSVTCTVSGVSLP | |
| | DYGVSWIRQPPRKGLEWLGVIWGSETTYYNSALKSRLTII | |
| | KDNSKSQVFLKMNSLQTDDTAIYYCAKHYYYGGSYAMD | |
| | YWGQGTSVTVSSIEVMYPPPYLDNEKSNGTIIHVKGKHL | |
| | CPSPLFPGPSKPFWVLVVVGGVLACYSLLVTVAFIIFWVR | |
| | SKRSRLLHSDYMNMTPRRPGPTRKHYQPYAPPRDFAAYR | |
| | SRVKFSRSADAPAYQQGQNQLYNELNLGRREEYDVLDK | |
| | RRGRDPEMGGKPRRKNPQEGLYNELQKDKMAEAYSEIG | |
| | MKGERRRGKGHDGLYQGLSTATKDTYDALHMQALPPR | |
| FMC63LC_E04_ | MARSPAQLLGLLLLWLSGARCDIQMTQTTSSLSASLGDR | 122 |
| E04_FMC63HC_ | VTISCRASQDISKYLNWYQQKPDGTVKLLIYHTSRLHSGV | |
| CD28_CD28_ | PSRFSGSGSGTDYSLTISNLEQEDIATYFCQQGNTLPYTFG | |
| CD28_CD3Z | GGTKLEITGSTSGSGKPGSGEGSTKGEVQLLESGGGLVQP | |
| (P1974) | GGSLRLSCAASGFTSSSYVMGWYRQAPGKEREFVSSISTG | |
| | GDAYYADSVKGRFTISRDNSKNTLYLQMNSLRAEDTAV | |
| | YYCAADVWYYHGGAYDYWGQGTQVTVSSGGGGSEVQ | |
| | LLESGGGLVQPGGSLRLSCAASGFTSSCYVMGWYRQAPG | |
| | KEREFVCTISTGGDAYYADSVKGRFTITRDNSKNTLYLQ | |
| | MNSLRAEDTAVYYCAADVWYYHGGAYDYWGQGTQVT | |
| | VSSGSTSGSGKPGSGEGSTKGEVKLQESGPGLVAPSQSLS | |
| | VTCTVSGVSLPDYGVSWIRQPPRKGLEWLGVIWGSETTY | |
| | YNSALKSRLTIIKDNSKSQVFLKMNSLQTDDTAIYYCAKH | |
| | YYYGGSYAMDYWGQGTSVTVSSIEVMYPPPYLDNEKSN | |
| | GTIIHVKGKHLCPSPLFPGPSKPFWVLVVVGGVLACYSLL | |
| | VTVAFIIFWVRSKRSRLLHSDYMNMTPRRPGPTRKHYQP | |
| | YAPPRDFAAYRSRVKFSRSADAPAYQQGQNQLYNELNL | |
| | GRREEYDVLDKRRGRDPEMGGKPRRKNPQEGLYNELQK | |
| | DKMAEAYSEIGMKGERRRGKGHDGLYQGLSTATKDTYD | |
| | ALHMQALPPR | |
| FMC63LC_E04_ | MARSPAQLLGLLLLWLSGARCDIQMTQTTSSLSASLGDR | 123 |
| A01_FMC63HC_ | VTISCRASQDISKYLNWYQQKPDGTVKLLIYHTSRLHSGV | |
| CD28_CD28_ | PSRFSGSGSGTDYSLTISNLEQEDIATYFCQQGNTLPYTFG | |
| CD28_CD3Z | GGTKLEITGSTSGSGKPGSGEGSTKGEVQLLESGGGLVQP | |
| (P2012) | GGSLRLSCAASGFTSSSYVMGWYRQAPGKEREFVSSISTG | |
| | GDAYYADSVKGRFTISRDNSKNTLYLQMNSLRAEDTAV | |
| | YYCAADVWYYHGGAYDYWGQGTQVTVSSGGGGSEVQ | |
| | LLESGGGLVQPGGTLRLSCAASGLTCYSYAMGWYRQAP | |
| | GKEREFVSAISSGGSAYYADSVKGRFTICRDNSKNTLYLQ | |
| | MNSLRAEDTAVYYCAVGPYYGFRAVTEADYWGQGTQV | |
| | TVSSGSTSGSGKPGSGEGSTKGEVKLQESGPGLVAPSQSL | |
| | SVTCTVSGVSLPDYGVSWIRQPPRKGLEWLGVIWGSETT | |
| | YYNSALKSRLTIIKDNSKSQVFLKMNSLQTDDTAIYYCAK | |
| | HYYYGGSYAMDYWGQGTSVTVSSIEVMYPPPYLDNEKS | |
| | NGTIIHVKGKHLCPSPLFPGPSKPFWVLVVVGGVLACYSL | |
| | LVTVAFIIFWVRSKRSRLLHSDYMNMTPRRPGPTRKHYQ | |
| | PYAPPRDFAAYRSRVKFSRSADAPAYQQGQNQLYNELNL | |
| | GRREEYDVLDKRRGRDPEMGGKPRRKNPQEGLYNELQK | |
| | DKMAEAYSEIGMKGERRRGKGHDGLYQGLSTATKDTYD | |
| | ALHMQALPPR | |
| FMC63LC_E04_ | MARSPAQLLGLLLLWLSGARCDIQMTQTTSSLSASLGDR | 124 |
| D04AMD11_ | VTISCRASQDISKYLNWYQQKPDGTVKLLIYHTSRLHSGV | |
| FMC63HC_ | PSRFSGSGSGTDYSLTISNLEQEDIATYFCQQGNTLPYTFG | |
| CD28_CD28_ | GGTKLEITGSTSGSGKPGSGEGSTKGEVQLLESGGGLVQP | |
| CD28_CD3Z | GGSLRLSCAASGFTSSSYVMGWYRQAPGKEREFVSSISTG | |
| (P2013) | GDAYYADSVKGRFTISRDNSKNTLYLQMNSLRAEDTAV | |
| | YYCAADVWYYHGGAYDYWGQGTQVTVSSGGGGSQVQ | |
| | LVESGGGLVQAGGSLRLSCAASGSEFTGYPMGWFRQAP | |
| | GKEREFVAGSVGIGGSTNYADSVKGRFTISRDNAKNTVY | |
| | LQMNSLKPEDTAVYYCAADKDYYKPYSRYRTAIRYDTW | |
| | GQGTQVTVSSGSTSGSGKPGSGEGSTKGEVKLQESGPGL | |
| | VAPSQSLSVTCTVSGVSLPDYGVSWIRQPPRKGLEWLGVI | |
| | WGSETTYYNSALKSRLTIIKDNSKSQVFLKMNSLQTDDT | |
| | AIYYCAKHYYYGGSYAMDYWGQGTSVTVSSIEVMYPPP | |
| | YLDNEKSNGTIIHVKGKHLCPSPLFPGPSKPFWVLVVVGG | |
| | VLACYSLLVTVAFIIFWVRSKRSRLLHSDYMNMTPRRPGP | |
| | TRKHYQPYAPPRDFAAYRSRVKFSRSADAPAYQQGQNQ | |
| | LYNELNLGRREEYDVLDKRRGRDPEMGGKPRRKNPQEG | |
| | LYNELQKDKMAEAYSEIGMKGERRRGKGHDGLYQGLST | |
| | ATKDTYDALHMQALPPR | |
| E04_FMC63_ | MARSPAQLLGLLLLWLSGARCEVQLLESGGGLVQPGGSL | 125 |
| CD28_CD28_ | RLSCAASGFTSSSYVMGWYRQAPGKEREFVSSISTGGDA | |
| CD28_CD3Z | YYADSVKGRFTISRDNSKNTLYLQMNSLRAEDTAVYYC | |
| (P1972) | AADVWYYHGGAYDYWGQGTQVTVSSGSTSGSGKPGSG | |
| | EGSTKGDIQMTQTTSSLSASLGDRVTISCRASQDISKYLN | |
| | WYQQKPDGTVKLLIYHTSRLHSGVPSRFSGSGSGTDYSLT | |
| | ISNLEQEDIATYFCQQGNTLPYTFGGGTKLEITGSTSGSGK | |
| | PGSGEGSTKGEVKLQESGPGLVAPSQSLSVTCTVSGVSLP | |
| | DYGVSWIRQPPRKGLEWLGVIWGSETTYYNSALKSRLTII | |
| | KDNSKSQVFLKMNSLQTDDTAIYYCAKHYYYGGSYAMD | |
| | YWGQGTSVTVSSIEVMYPPPYLDNEKSNGTIIHVKGKHL | |
| | CPSPLFPGPSKPFWVLVVVGGVLACYSLLVTVAFIIFWVR | |
| | SKRSRLLHSDYMNMTPRRPGPTRKHYQPYAPPRDFAAYR | |
| | SRVKFSRSADAPAYQQGQNQLYNELNLGRREEYDVLDK | |
| | RRGRDPEMGGKPRRKNPQEGLYNELQKDKMAEAYSEIG | |
| | MKGERRRGKGHDGLYQGLSTATKDTYDALHMQALPPR | |
| A01_FMC63_ | MARSPAQLLGLLLLWLSGARCEVQLLESGGGLVQPGGSL | 126 |
| CD28_CD28_ | RLSCAASGLTSYSYAMGWYRQAPGKEREFVSAISSGGSA | |
| CD28_CD3Z | YYADSVKGRFTISRDNSKNTLYLQMNSLRAEDTAVYYC | |
| (P2014) | AVGPYYGFRAVTEADYWGQGTQVTVSSGSTSGSGKPGS | |
| | GEGSTKGDIQMTQTTSSLSASLGDRVTISCRASQDISKYL | |
| | NWYQQKPDGTVKLLIYHTSRLHSGVPSRFSGSGSGTDYS | |
| | LTISNLEQEDIATYFCQQGNTLPYTFGGGTKLEITGSTSGS | |
| | GKPGSGEGSTKGEVKLQESGPGLVAPSQSLSVTCTVSGVS | |
| | LPDYGVSWIRQPPRKGLEWLGVIWGSETTYYNSALKSRL | |
| | TIIKDNSKSQVFLKMNSLQTDDTAIYYCAKHYYYGGSYA | |
| | MDYWGQGTSVTVSSIEVMYPPPYLDNEKSNGTIIHVKGK | |
| | HLCPSPLFPGPSKPFWVLVVVGGVLACYSLLVTVAFIIFW | |
| | VRSKRSRLLHSDYMNMTPRRPGPTRKHYQPYAPPRDFAA | |
| | YRSRVKFSRSADAPAYQQGQNQLYNELNLGRREEYDVL | |
| | DKRRGRDPEMGGKPRRKNPQEGLYNELQKDKMAEAYSE | |
| | IGMKGERRRGKGHDGLYQGLSTATKDTYDALHMQALPP | |
| | R | |
| D04AMD11_E04_ | MARSPAQLLGLLLLWLSGARCQVQLVESGGGLVQAGGS | 127 |
| FMC63_CD28_ | LRLSCAASGSEFTGYPMGWFRQAPGKEREFVAGSVGIGG | |
| CD28_CD28_ | STNYADSVKGRFTISRDNAKNTVYLQMNSLKPEDTAVYY | |
| CD3Z | CAADKDYYKPYSRYRTAIRYDTWGQGTQVTVSSGGGGS | |
| (P2015) | EVQLLESGGGLVQPGGSLRLSCAASGFTSSSYVMGWYRQ | |
| | APGKEREFVSSISTGGDAYYADSVKGRFTISRDNSKNTLY | |
| | LQMNSLRAEDTAVYYCAADVWYYHGGAYDYWGQGTQ | |
| | VTVSSGSTSGSGKPGSGEGSTKGDIQMTQTTSSLSASLGD | |
| | RVTISCRASQDISKYLNWYQQKPDGTVKLLIYHTSRLHSG | |
| | VPSRFSGSGSGTDYSLTISNLEQEDIATYFCQQGNTLPYTF | |
| | GGGTKLEITGSTSGSGKPGSGEGSTKGEVKLQESGPGLVA | |
| | PSQSLSVTCTVSGVSLPDYGVSWIRQPPRKGLEWLGVIW | |
| | GSETTYYNSALKSRLTIIKDNSKSQVFLKMNSLQTDDTAI | |
| | YYCAKHYYYGGSYAMDYWGQGTSVTVSSIEVMYPPPYL | |
| | DNEKSNGTIIHVKGKHLCPSPLFPGPSKPFWVLVVVGGVL | |
| | ACYSLLVTVAFIIFWVRSKRSRLLHSDYMNMTPRRPGPTR | |
| | KHYQPYAPPRDFAAYRSRVKFSRSADAPAYQQGQNQLY | |
| | NELNLGRREEYDVLDKRRGRDPEMGGKPRRKNPQEGLY | |
| | NELQKDKMAEAYSEIGMKGERRRGKGHDGLYQGLSTAT | |
| | KDTYDALHMQALPPR | |
| D04_E04_A01_ | MARSPAQLLGLLLLWLSGARCQVQLVESGGGLVQAGGS | 128 |
| D04_FMC63_ | LRLSCAASGSEFTGYPMGWFRQAPGKEREFVAGSVGIGG | |
| CD28_CD28_ | STNYADSVKGRFTISRDNAKNTVYLQMNSLKPEDTAVYY | |
| CD28_CD3Z | CAADKDYYKPYSRYRTAIRYDTWGQGTQVTVSSGGGGS | |
| (P2016) | EVQLLESGGGLVQPGGSLRLSCAASGFTSSSYVMGWYRQ | |
| | APGKEREFVSSISTGGDAYYADSVKGRFTISRDNSKNTLY | |
| | LQMNSLRAEDTAVYYCAADVWYYHGGAYDYWGQGTQ | |
| | VTVSSGGGGSEVQLLESGGGLVQPGGTLRLSCAASGLTC | |
| | YSYAMGWYRQAPGKEREFVSAISSGGSAYYADSVKGRF | |
| | TICRDNSKNTLYLQMNSLRAEDTAVYYCAVGPYYGFRA | |
| | VTEADYWGQGTQVTVSSGGGGSQVQLVESGGGLVQAG | |
| | GSLRLSCAASGSEFTGYPMGWFRQAPGKEREFVAGSVGI | |
| | GGSTNYADSVKGRFTISRDNAKNTVYLQMNSLKPEDTAV | |
| | YYCAADKDYYKPYSRYRTAIRYDTWGQGTQVTVSSGST | |
| | SGSGKPGSGEGSTKGDIQMTQTTSSLSASLGDRVTISCRA | |
| | SQDISKYLNWYQQKPDGTVKLLIYHTSRLHSGVPSRFSGS | |
| | GSGTDYSLTISNLEQEDIATYFCQQGNTLPYTFGGGTKLEI | |
| | TGSTSGSGKPGSGEGSTKGEVKLQESGPGLVAPSQSLSVT | |
| | CTVSGVSLPDYGVSWIRQPPRKGLEWLGVIWGSETTYYN | |
| | SALKSRLTIIKDNSKSQVFLKMNSLQTDDTAIYYCAKHYY | |
| | YGGSYAMDYWGQGTSVTVSSIEVMYPPPYLDNEKSNGTI | |
| | IHVKGKHLCPSPLFPGPSKPFWVLVVVGGVLACYSLLVT | |
| | VAFIIFWVRSKRSRLLHSDYMNMTPRRPGPTRKHYQPYA | |
| | PPRDFAAYRSRVKFSRSADAPAYQQGQNQLYNELNLGRR | |
| | EEYDVLDKRRGRDPEMGGKPRRKNPQEGLYNELQKDK | |
| | MAEAYSEIGMKGERRRGKGHDGLYQGLSTATKDTYDAL | |
| | HMQALPPR | |
| FMC63LC_A01_ | MARSPAQLLGLLLLWLSGARCDIQMTQTTSSLSASLGDR | 129 |
| A01_FMC63HC_ | VTISCRASQDISKYLNWYQQKPDGTVKLLIYHTSRLHSGV | |
| CD28_CD28_ | PSRFSGSGSGTDYSLTISNLEQEDIATYFCQQGNTLPYTFG | |
| CD28_CD3Z | GGTKLEITGSTSGSGKPGSGEGSTKGEVQLLESGGGLVQP | |
| (P2193) | GGSLRLSCAASGLTSYSYAMGWYRQAPGKEREFVSAISS | |
| | GGSAYYADSVKGRFTISRDNSKNTLYLQMNSLRAEDTAV | |
| | YYCAVGPYYGFRAVTEADYWGQGTQVTVSSGGGGSEV | |
| | QLLESGGGLVQPGGTLRLSCAASGLTCYSYAMGWYRQA | |
| | PGKEREFVSAISSGGSAYYADSVKGRFTICRDNSKNTLYL | |
| | QMNSLRAEDTAVYYCAVGPYYGFRAVTEADYWGQGTQ | |
| | VTVSSGSTSGSGKPGSGEGSTKGEVKLQESGPGLVAPSQS | |
| | LSVTCTVSGVSLPDYGVSWIRQPPRKGLEWLGVIWGSET | |
| | TYYNSALKSRLTIIKDNSKSQVFLKMNSLQTDDTAIYYCA | |
| | KHYYYGGSYAMDYWGQGTSVTVSSIEVMYPPPYLDNEK | |
| | SNGTIIHVKGKHLCPSPLFPGPSKPFWVLVVVGGVLACYS | |
| | LLVTVAFIIFWVRSKRSRLLHSDYMNMTPRRPGPTRKHY | |
| | QPYAPPRDFAAYRSRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKDT | |
| | YDALHMQALPPR | |
| FMC63LC_ | MARSPAQLLGLLLLWLSGARCDIQMTQTTSSLSASLGDR | 130 |
| D04AMD11_A01_ | VTISCRASQDISKYLNWYQQKPDGTVKLLIYHTSRLHSGV | |
| FMC63HC_ | PSRFSGSGSGTDYSLTISNLEQEDIATYFCQQGNTLPYTFG | |
| CD28_CD28_ | GGTKLEITGSTSGSGKPGSGEGSTKGQVQLVESGGGLVQ | |
| CD28_CD3Z | AGGSLRLSCAASGSEFTGYPMGWFRQAPGKEREFVAGSV | |
| (P2191) | GIGGSTNYADSVKGRFTISRDNAKNTVYLQMNSLKPEDT | |
| | AVYYCAADKDYYKPYSRYRTAIRYDTWGQGTQVTVSSG | |
| | GGGSEVQLLESGGGLVQPGGTLRLSCAASGLTCYSYAMG | |
| | WYRQAPGKEREFVSAISSGGSAYYADSVKGRFTICRDNS | |
| | KNTLYLQMNSLRAEDTAVYYCAVGPYYGFRAVTEADY | |
| | WGQGTQVTVSSGSTSGSGKPGSGEGSTKGEVKLQESGPG | |
| | LVAPSQSLSVTCTVSGVSLPDYGVSWIRQPPRKGLEWLG | |
| | VIWGSETTYYNSALKSRLTIIKDNSKSQVFLKMNSLQTDD | |
| | TAIYYCAKHYYYGGSYAMDYWGQGTSVTVSSIEVMYPP | |
| | PYLDNEKSNGTIIHVKGKHLCPSPLFPGPSKPFWVLVVVG | |
| | GVLACYSLLVTVAFIIFWVRSKRSRLLHSDYMNMTPRRP | |
| | GPTRKHYQPYAPPRDFAAYRSRVKFSRSADAPAYQQGQ | |
| | NQLYNELNLGRREEYDVLDKRRGRDPEMGGKPRRKNPQ | |
| | EGLYNELQKDKMAEAYSEIGMKGERRRGKGHDGLYQGL | |
| | STATKDTYDALHMQALPPR | |
| FMC63LC_A01_ | MARSPAQLLGLLLLWLSGARCDIQMTQTTSSLSASLGDR | 131 |
| D04AMD11_ | VTISCRASQDISKYLNWYQQKPDGTVKLLIYHTSRLHSGV | |
| A01_FMC63HC_ | PSRFSGSGSGTDYSLTISNLEQEDIATYFCQQGNTLPYTFG | |
| CD28_CD28_ | GGTKLEITGSTSGSGKPGSGEGSTKGEVQLLESGGGLVQP | |
| CD28_CD3Z | GGSLRLSCAASGLTSYSYAMGWYRQAPGKEREFVSAISS | |
| (P2195) | GGSAYYADSVKGRFTISRDNSKNTLYLQMNSLRAEDTAV | |
| | YYCAVGPYYGFRAVTEADYWGQGTQVTVSSGGGGSQV | |
| | QLVESGGGLVQAGGSLRLSCAASGSEFTGYPMGWFRQA | |
| | PGKEREFVAGSVGIGGSTNYADSVKGRFTISRDNAKNTV | |
| | YLQMNSLKPEDTAVYYCAADKDYYKPYSRYRTAIRYDT | |
| | WGQGTQVTVSSGGGGSEVQLLESGGGLVQPGGTLRLSC | |
| | AASGLTCYSYAMGWYRQAPGKEREFVSAISSGGSAYYA | |
| | DSVKGRFTICRDNSKNTLYLQMNSLRAEDTAVYYCAVGP | |
| | YYGFRAVTEADYWGQGTQVTVSSGSTSGSGKPGSGEGST | |
| | KGEVKLQESGPGLVAPSQSLSVTCTVSGVSLPDYGVSWIR | |
| | QPPRKGLEWLGVIWGSETTYYNSALKSRLTIIKDNSKSQV | |
| | FLKMNSLQTDDTAIYYCAKHYYYGGSYAMDYWGQGTS | |
| | VTVSSIEVMYPPPYLDNEKSNGTIIHVKGKHLCPSPLFPGP | |
| | SKPFWVLVVVGGVLACYSLLVTVAFIIFWVRSKRSRLLHS | |
| | DYMNMTPRRPGPTRKHYQPYAPPRDFAAYRSRVKFSRSA | |
| | DAPAYQQGQNQLYNELNLGRREEYDVLDKRRGRDPEM | |
| | GGKPRRKNPQEGLYNELQKDKMAEAYSEIGMKGERRRG | |
| | KGHDGLYQGLSTATKDTYDALHMQALPPR | |

Polynucleotide Sequences of Bi-specific CARs Targeting CD19 and CD22:

| | | |
|---|---|---|
| FMC63LC_E04_ | ATGGCCAGATCTCCTGCTCAACTGCTGGGACTGCTGCT | 132 |
| FMC63HC_ | GCTGTGGCTTAGCGGAGCCAGATGCGACATCCAGATG | |
| CD28_CD28_CD28_ | ACCCAGACCACAAGCAGCCTGTCTGCCAGCCTGGGCG | |
| CD3Z | ATAGAGTGACCATCAGCTGTAGAGCCAGCCAGGACAT | |
| (P1973) | CAGCAAGTACCTGAACTGGTATCAGCAAAAGCCCGAC | |
| | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTTACCAGCT | |
| | CCTCCTACGTGATGGGCTGGTATCGCCAAGCGCCGGGC | |
| | AAAGAACGCGAGTTTGTCAGCTCGATCAGCACCGGTG | |
| | GTGATGCCTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGCTGACGTTTGGTACTACCACGGCGG | |
| | CGCGTACGATTATTGGGGCCAGGGTACCCAGGTGACG | |
| | GTCTCGAGCGGCAGTACTTCTGGTAGCGGAAAACCCG | |
| | GTAGCGGCGAGGGGTCAACTAAAGGAGAAGTGAAACT | |
| | GCAAGAGTCTGGCCCTGGACTGGTGGCCCCATCTCAGT | |
| | CTCTGAGCGTGACCTGTACAGTCAGCGGAGTGTCCCTG | |
| | CCTGATTACGGCGTGTCCTGGATCAGACAGCCTCCTCG | |
| | GAAAGGCCTGGAATGGCTGGGAGTGATCTGGGGCAGC | |
| | GAGACAACCTACTACAACAGCGCCCTGAAGTCCCGGC | |
| | TGACCATCATCAAGGACAACTCCAAGAGCCAGGTGTTC | |
| | CTGAAGATGAACAGCCTGCAGACCGACGACACCGCCA | |
| | TCTACTATTGCGCCAAGCACTACTACTACGGCGGCAGC | |
| | TACGCCATGGATTATTGGGGCCAGGGCACCAGCGTGA | |
| | CCGTGTCTAGCATCGAAGTGATGTACCCTCCACCTTAC | |
| | CTGGACAACGAGAAGTCCAACGGCACCATCATCCACG | |
| | TGAAGGGCAAGCACCTGTGTCCTTCTCCACTGTTCCCC | |
| | GGACCTAGCAAGCCTTTCTGGGTGCTCGTTGTTGTTGG | |
| | CGGCGTGCTGGCCTGTTATAGCCTGCTTGTGACCGTGG | |
| | CCTTCATCATCTTTTGGGTCCGAAGCAAGCGGAGCCGG | |
| | CTGCTGCACTCCGACTACATGAACATGACCCCTAGACG | |
| | GCCCGGACCAACCAGAAAGCACTACCAGCCTTACGCT | |
| | CCTCCTAGAGACTTCGCCGCCTACCGGTCCAGAGTGAA | |
| | GTTCAGCAGGAGCGCAGACGCCCCCGCGTACCAGCAG | |
| | GGCCAGAACCAGCTCTATAACGAGCTCAATCTAGGAC | |
| | GAAGAGAGGAGTACGATGTTTTGGACAAGCGACGTGG | |
| | CCGGGACCCTGAGATGGGGGGAAAGCCGAGAAGGAA | |
| | GAACCCTCAGGAAGGCCTGTACAATGAACTGCAGAAA | |
| | GATAAGATGGCGGAGGCCTACAGTGAGATTGGGATGA | |
| | AAGGCGAGCGCCGGAGGGGCAAGGGGCACGATGGCCT | |
| | TTACCAGGGACTCAGTACAGCCACCAAGGACACCTAC | |
| | GACGCCCTTCACATGCAGGCCCTGCCCCCTCGC | |
| FMC63LC_A01_ | ATGGCCAGATCTCCTGCTCAACTGCTGGGACTGCTGCT | 133 |
| FMC63HC_ | GCTGTGGCTTAGCGGAGCCAGATGCGACATCCAGATG | |
| CD28_CD28_ | ACCCAGACCACAAGCAGCCTGTCTGCCAGCCTGGGCG | |
| CD28_CD3Z | ATAGAGTGACCATCAGCTGTAGAGCCAGCCAGGACAT | |
| (P1988) | CAGCAAGTACCTGAACTGGTATCAGCAAAAGCCCGAC | |
| | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTGACCTCTT | |
| | ATTCCTACGCGATGGGCTGGTATCGCCAAGCGCCGGGC | |
| | AAAGAACGCGAGTTTGTCAGCGCAATCAGCTCGGGTG | |
| | GTAGCGCGTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGTTGGACCGTACTACGGATTTAGAGC | |
| | GGTTACCGAAGCAGATTATTGGGGCCAGGGTACCCAG | |
| | GTGACGGTCTCGAGCGGCAGTACTTCTGGTAGCGGAA | |
| | AACCCGGTAGCGGCGAGGGGTCAACTAAAGGAGAAGT | |
| | GAAACTGCAAGAGTCTGGCCCTGGACTGGTGGCCCCAT | |
| | CTCAGTCTCTGAGCGTGACCTGTACAGTCAGCGGAGTG | |
| | TCCCTGCCTGATTACGGCGTGTCCTGGATCAGACAGCC | |
| | TCCTCGGAAAGGCCTGGAATGGCTGGGAGTGATCTGG | |
| | GGCAGCGAGACAACCTACTACAACAGCGCCCTGAAGT | |
| | CCCGGCTGACCATCATCAAGGACAACTCCAAGAGCCA | |
| | GGTGTTCCTGAAGATGAACAGCCTGCAGACCGACGAC | |
| | ACCGCCATCTACTATTGCGCCAAGCACTACTACTACGG | |
| | CGGCAGCTACGCCATGGATTATTGGGGCCAGGGCACC | |
| | AGCGTGACCGTGTCTAGCATCGAAGTGATGTACCCTCC | |
| | ACCTTACCTGGACAACGAGAAGTCCAACGGCACCATC | |
| | ATCCACGTGAAGGGCAAGCACCTGTGTCCTTCTCCACT | |
| | GTTCCCCGGACCTAGCAAGCCTTTCTGGGTGCTCGTTG | |
| | TTGTTGGCGGCGTGCTGGCCTGTTATAGCCTGCTTGTG | |
| | ACCGTGGCCTTCATCATCTTTTGGGTCCGAAGCAAGCG | |
| | GAGCCGGCTGCTGCACTCCGACTACATGAACATGACCC | |
| | CTAGACGGCCCGGACCAACCAGAAAGCACTACCAGCC | |
| | TTACGCTCCTCCTAGAGACTTCGCCGCCTACCGGTCCA | |
| | GAGTGAAGTTCAGCAGGAGCGCAGACGCCCCCGCGTA | |
| | CCAGCAGGGCCAGAACCAGCTCTATAACGAGCTCAAT | |
| | CTAGGACGAAGAGAGGAGTACGATGTTTTGGACAAGC | |
| | GACGTGGCCGGGACCCTGAGATGGGGGGAAAGCCGAG | |
| | AAGGAAGAACCCTCAGGAAGGCCTGTACAATGAACTG | |
| | CAGAAAGATAAGATGGCGGAGGCCTACAGTGAGATTG | |
| | GGATGAAAGGCGAGCGCCGGAGGGGCAAGGGGCACG | |
| | ATGGCCTTTACCAGGGACTCAGTACAGCCACCAAGGA | |
| | CACCTACGACGCCCTTCACATGCAGGCCCTGCCCCCTC | |
| | GC | |
| FMC63LC_E04_ | ATGGCCAGATCTCCTGCTCAACTGCTGGGACTGCTGCT | 134 |
| E04_FMC63HC_ | GCTGTGGCTTAGCGGAGCCAGATGCGACATCCAGATG | |
| CD28_CD28_ | ACCCAGACCACAAGCAGCCTGTCTGCCAGCCTGGGCG | |
| CD28_CD3Z | ATAGAGTGACCATCAGCTGTAGAGCCAGCCAGGACAT | |
| (P1974) | CAGCAAGTACCTGAACTGGTATCAGCAAAAGCCCGAC | |
| | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTTACCAGCT | |
| | CCTCCTACGTGATGGGCTGGTATCGCCAAGCGCCGGGC | |
| | AAAGAACGCGAGTTTGTCAGCTCGATCAGCACCGGTG | |
| | GTGATGCCTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGCTGACGTTTGGTACTACCACGGCGG | |
| | CGCGTACGATTATTGGGGCCAGGGTACCCAGGTGACG | |
| | GTCTCGAGCGGCGGTGGCGGATCAGAAGTCCAGCTGC | |
| | TGGAAAGCGGTGGCGGTCTGGTCCAGCCCGGCGGCAG | |
| | CCTGCGCCTGTCCTGTGCCGCTAGCGGTTTCACTTCCA | |
| | GCTGCTATGTCATGGGTTGGTACCGCCAGGCCCCCGGT | |
| | AAGGAGCGCGAATTCGTGTGCACCATTTCCACTGGCGG | |
| | CGACGCTTATTATGCTGATAGCGTGAAGGGTCGCTTCA | |
| | CTATTACCCGCGACAACAGCAAAAACACTCTGTATCTG | |
| | CAGATGAACTCCCTGCGCGCTGAGGATACCGCCGTCTA | |
| | CTACTGCGCTGCCGATGTGTGGTATTATCATGGTGGTG | |
| | CCTATGACTACTGGGGTCAGGGCACTCAGGTCACCGTC | |
| | AGCTCCGGCAGTACTTCTGGTAGCGGAAAACCCGGTA | |
| | GCGGCGAGGGGTCAACTAAAGGAGAAGTGAAACTGCA | |
| | AGAGTCTGGCCCTGGACTGGTGGCCCCATCTCAGTCTC | |
| | TGAGCGTGACCTGTACAGTCAGCGGAGTGTCCCTGCCT | |
| | GATTACGGCGTGTCCTGGATCAGACAGCCTCCTCGGAA | |
| | AGGCCTGGAATGGCTGGGAGTGATCTGGGGCAGCGAG | |
| | ACAACCTACTACAACAGCGCCCTGAAGTCCCGGCTGAC | |
| | CATCATCAAGGACAACTCCAAGAGCCAGGTGTTCCTGA | |
| | AGATGAACAGCCTGCAGACCGACGACACCGCCATCTA | |
| | CTATTGCGCCAAGCACTACTACTACGGCGGCAGCTACG | |
| | CCATGGATTATTGGGGCCAGGGCACCAGCGTGACCGT | |
| | GTCTAGCATCGAAGTGATGTACCCTCCACCTTACCTGG | |
| | ACAACGAGAAGTCCAACGGCACCATCATCCACGTGAA | |
| | GGGCAAGCACCTGTGTCCTTCTCCACTGTTCCCCGGAC | |
| | CTAGCAAGCCTTTCTGGGTGCTCGTTGTTGTTGGCGGC | |
| | GTGCTGGCCTGTTATAGCCTGCTTGTGACCGTGGCCTT | |
| | CATCATCTTTTGGGTCCGAAGCAAGCGGAGCCGGCTGC | |
| | TGCACTCCGACTACATGAACATGACCCCTAGACGGCCC | |
| | GGACCAACCAGAAAGCACTACCAGCCTTACGCTCCTCC | |
| | TAGAGACTTCGCCGCCTACCGGTCCAGAGTGAAGTTCA | |
| | GCAGGAGCGCAGACGCCCCCGCGTACCAGCAGGGCCA | |
| | GAACCAGCTCTATAACGAGCTCAATCTAGGACGAAGA | |
| | GAGGAGTACGATGTTTTGGACAAGCGACGTGGCCGGG | |
| | ACCCTGAGATGGGGGGAAAGCCGAGAAGGAAGAACCC | |
| | TCAGGAAGGCCTGTACAATGAACTGCAGAAAGATAAG | |
| | ATGGCGGAGGCCTACAGTGAGATTGGGATGAAAGGCG | |
| | AGCGCCGGAGGGGCAAGGGGCACGATGGCCTTTACCA | |
| | GGGACTCAGTACAGCCACCAAGGACACCTACGACGCC | |
| | CTTCACATGCAGGCCCTGCCCCCTCGC | |
| FMC63LC_E04_ | ATGGCCAGATCTCCTGCTCAACTGCTGGGACTGCTGCT | 135 |
| A01_FMC63HC_ | GCTGTGGCTTAGCGGAGCCAGATGCGACATCCAGATG | |
| CD28_CD28_ | ACCCAGACCACAAGCAGCCTGTCTGCCAGCCTGGGCG | |
| CD28_CD3Z | ATAGAGTGACCATCAGCTGTAGAGCCAGCCAGGACAT | |
| (P2012) | CAGCAAGTACCTGAACTGGTATCAGCAAAAGCCCGAC | |
| | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTTACCAGCT | |
| | CCTCCTACGTGATGGGCTGGTATCGCCAAGCGCCGGGC | |
| | AAAGAACGCGAGTTTGTCAGCTCGATCAGCACCGGTG | |
| | GTGATGCCTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGCTGACGTTTGGTACTACCACGGCGG | |
| | CGCGTACGATTATTGGGGCCAGGGTACCCAGGTGACG | |
| | GTCTCGAGCGGCGGTGGCGGATCAGAAGTCCAGCTGC | |
| | TGGAAAGCGGTGGCGGTCTGGTCCAGCCTGGCGGCAC | |
| | CCTGCGCCTGTCCTGTGCCGCTAGCGGCCTGACCTGCT | |
| | ATAGCTATGCCATGGGTTGGTACCGCCAGGCCCCTGGT | |
| | AAGGAGCGCGAATTCGTGTCCGCTATTTCCAGCGGCGG | |
| | CTCCGCCTATTATGCTGATAGCGTCAAGGGTCGCTTCA | |
| | CCATTTGCCGCGACAACAGCAAAAACACTCTGTATCTG | |
| | CAGATGAACTCCCTGCGCGCTGAGGATACCGCCGTCTA | |
| | CTACTGCGCTGTGGGCCCTTATTATGGCTTCCGCGCTGT | |
| | GACTGAGGCTGACTACTGGGGTCAGGGCACTCAGGTG | |
| | ACTGTGAGCAGCGGCAGTACTTCTGGTAGCGGAAAAC | |
| | CCGGTAGCGGCGAGGGGTCAACTAAAGGAGAAGTGAA | |
| | ACTGCAAGAGTCTGGCCCTGGACTGGTGGCCCCATCTC | |
| | AGTCTCTGAGCGTGACCTGTACAGTCAGCGGAGTGTCC | |
| | CTGCCTGATTACGGCGTGTCCTGGATCAGACAGCCTCC | |
| | TCGGAAAGGCCTGGAATGGCTGGGAGTGATCTGGGGC | |
| | AGCGAGACAACCTACTACAACAGCGCCCTGAAGTCCC | |
| | GGCTGACCATCATCAAGGACAACTCCAAGAGCCAGGT | |
| | GTTCCTGAAGATGAACAGCCTGCAGACCGACGACACC | |
| | GCCATCTACTATTGCGCCAAGCACTACTACTACGGCGG | |
| | CAGCTACGCCATGGATTATTGGGGCCAGGGCACCAGC | |
| | GTGACCGTGTCTAGCATCGAAGTGATGTACCCTCCACC | |
| | TTACCTGGACAACGAGAAGTCCAACGGCACCATCATCC | |
| | ACGTGAAGGGCAAGCACCTGTGTCCTTCTCCACTGTTC | |
| | CCCGGACCTAGCAAGCCTTTCTGGGTGCTCGTTGTTGT | |
| | TGGCGGCGTGCTGGCCTGTTATAGCCTGCTTGTGACCG | |
| | TGGCCTTCATCATCTTTTGGGTCCGAAGCAAGCGGAGC | |
| | CGGCTGCTGCACTCCGACTACATGAACATGACCCCTAG | |
| | ACGGCCCGGACCAACCAGAAAGCACTACCAGCCTTAC | |
| | GCTCCTCCTAGAGACTTCGCCGCCTACCGGTCCAGAGT | |
| | GAAGTTCAGCAGATCCGCCGATGCTCCCGCCTATCAGC | |
| | AGGGCCAAAACCAGCTGTACAACGAGCTGAACCTGGG | |
| | GAGAAGAGAAGAGTACGACGTGCTGGACAAGCGGAG | |
| | AGGCAGAGATCCTGAAATGGGCGGCAAGCCCAGACGG | |
| | AAGAATCCTCAAGAGGGCCTGTATAATGAGCTGCAGA | |
| | AAGACAAGATGGCCGAGGCCTACAGCGAGATCGGAAT | |
| | GAAGGGCGAGCGCAGAAGAGGCAAGGGACACGATGG | |
| | ACTGTACCAGGGCCTGAGCACCGCCACCAAGGATACC | |
| | TATGATGCCCTGCACATGCAGGCCCTGCCTCCAAGA | |
| FMC63LC_E04_ | ATGGCCAGATCTCCTGCTCAACTGCTGGGACTGCTGCT | 136 |
| D04AMD11_ | GCTGTGGCTTAGCGGAGCCAGATGCGACATCCAGATG | |
| FMC63HC_ | ACCCAGACCACAAGCAGCCTGTCTGCCAGCCTGGGCG | |
| CD28_CD28_ | ATAGAGTGACCATCAGCTGTAGAGCCAGCCAGGACAT | |
| CD28_CD3Z | CAGCAAGTACCTGAACTGGTATCAGCAAAAGCCCGAC | |
| (P2013) | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTTACCAGCT | |
| | CCTCCTACGTGATGGGCTGGTATCGCCAAGCGCCGGGC | |
| | AAAGAACGCGAGTTTGTCAGCTCGATCAGCACCGGTG | |
| | GTGATGCCTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGCTGACGTTTGGTACTACCACGGCGG | |
| | CGCGTACGATTATTGGGGCCAGGGTACCCAGGTGACG | |
| | GTCTCGAGCGGCGGTGGCGGATCACAGGTGCAGCTGG | |
| | TTGAGTCTGGGGGAGGCCTTGTCCAGGCTGGGGGGTCC | |
| | CTGAGACTCTCCTGTGCAGCGTCTGGAAGCGAATTCAC | |
| | CGGTTATCCCATGGGCTGGTTTCGCCAGGCTCCAGGCA | |
| | AGGAAAGGGAGTTTGTCGCTGGCTCCGTAGGTATCGGT | |
| | GGTAGTACAAACTATGCAGACTCCGTGAAGGGCCGAT | |
| | TCACCATCTCCAGAGACAATGCGAAGAACACGGTCTAT | |
| | CTGCAAATGAACAGCCTGAAGCCAGAGGACACGGCTG | |
| | TGTATTACTGTGCGGCCGACAAAGACTACTACAAACCT | |
| | TATAGTCGATATAGGACCGCTATCAGGTACGATACCTG | |
| | GGGCCAAGGGACCCAGGTCACCGTCTCGAGCGGCAGT | |
| | ACTTCTGGTAGCGGAAAACCCGGTAGCGGCGAGGGGT | |
| | CAACTAAAGGAGAAGTGAAACTGCAAGAGTCTGGCCC | |
| | TGGACTGGTGGCCCCATCTCAGTCTCTGAGCGTGACCT | |
| | GTACAGTCAGCGGAGTGTCCCTGCCTGATTACGGCGTG | |
| | TCCTGGATCAGACAGCCTCCTCGGAAAGGCCTGGAATG | |
| | GCTGGGAGTGATCTGGGGCAGCGAGACAACCTACTAC | |
| | AACAGCGCCCTGAAGTCCCGGCTGACCATCATCAAGG | |
| | ACAACTCCAAGAGCCAGGTGTTCCTGAAGATGAACAG | |
| | CCTGCAGACCGACGACACCGCCATCTACTATTGCGCCA | |
| | AGCACTACTACTACGGCGGCAGCTACGCCATGGATTAT | |
| | TGGGGCCAGGGCACCAGCGTGACCGTGTCTAGCATCG | |
| | AAGTGATGTACCCTCCACCTTACCTGGACAACGAGAAG | |
| | TCCAACGGCACCATCATCCACGTGAAGGGCAAGCACC | |
| | TGTGTCCTTCTCCACTGTTCCCCGGACCTAGCAAGCCTT | |
| | TCTGGGTGCTCGTTGTTGTTGGCGGCGTGCTGGCCTGTT | |
| | ATAGCCTGCTTGTGACCGTGGCCTTCATCATCTTTTGGG | |
| | TCCGAAGCAAGCGGAGCCGGCTGCTGCACTCCGACTA | |
| | CATGAACATGACCCCTAGACGGCCCGGACCAACCAGA | |
| | AAGCACTACCAGCCTTACGCTCCTCCTAGAGACTTCGC | |
| | CGCCTACCGGTCCAGAGTGAAGTTCAGCAGATCCGCCG | |
| | ATGCTCCCGCCTATCAGCAGGGCCAAAACCAGCTGTAC | |
| | AACGAGCTGAACCTGGGGAGAAGAGAAGAGTACGACG | |
| | TGCTGGACAAGCGGAGAGGCAGAGATCCTGAAATGGG | |
| | CGGCAAGCCCAGACGGAAGAATCCTCAAGAGGGCCTG | |
| | TATAATGAGCTGCAGAAAGACAAGATGGCCGAGGCCT | |
| | ACAGCGAGATCGGAATGAAGGGCGAGCGCAGAAGAG | |
| | GCAAGGGACACGATGGACTGTACCAGGGCCTGAGCAC | |
| | CGCCACCAAGGATACCTATGATGCCCTGCACATGCAGG | |
| | CCCTGCCTCCAAGA | |
| E04_FMC63_ | ATGGCCAGATCTCCTGCTCAACTGCTGGGACTGCTGCT | 137 |
| CD28_CD28_ | GCTGTGGCTTAGCGGAGCCAGATGCGAGGTACAACTTT | |
| CD28_CD3Z | TGGAGTCAGGCGGTGGACTGGTACAACCGGGTGGTTC | |
| (P1972) | ATTGCGTTTGAGCTGCGCTGCCTCTGGTTTTACCAGCTC | |
| | CTCCTACGTGATGGGCTGGTATCGCCAAGCGCCGGGCA | |
| | AAGAACGCGAGTTTGTCAGCTCGATCAGCACCGGTGGT | |
| | GATGCCTACTACGCGGACTCGGTAAAAGGCCGTTTTAC | |
| | GATCAGTCGTGATAATTCCAAGAATACCTTGTACCTGC | |
| | AAATGAATAGCCTTCGCGCAGAAGACACAGCGGTGTA | |
| | TTATTGTGCCGCTGACGTTTGGTACTACCACGGCGGCG | |
| | CGTACGATTATTGGGGCCAGGGTACCCAGGTGACGGTC | |
| | TCGAGCGGCAGTACTTCTGGTAGCGGAAAACCCGGTA | |
| | GCGGCGAGGGGTCAACTAAAGGAGACATCCAGATGAC | |
| | CCAGACCACAAGCAGCCTGTCTGCCAGCCTGGGCGAT | |
| | AGAGTGACCATCAGCTGTAGAGCCAGCCAGGACATCA | |
| | GCAAGTACCTGAACTGGTATCAGCAAAAGCCCGACGG | |
| | CACCGTGAAGCTGCTGATCTACCACACCAGCAGACTGC | |
| | ACAGCGGCGTGCCAAGCAGATTTTCTGGCAGCGGCTCT | |
| | GGCACCGACTACAGCCTGACAATCAGCAACCTGGAAC | |
| | AAGAGGATATCGCTACCTACTTCTGCCAGCAAGGCAAC | |
| | ACCCTGCCTTACACCTTTGGCGGAGGCACCAAGCTGGA | |
| | AATCACCGGCTCTACAAGCGGCAGCGGCAAACCTGGA | |
| | TCTGGCGAGGGATCTACCAAGGGCGAAGTGAAACTGC | |
| | AAGAGTCTGGCCCTGGACTGGTGGCCCCATCTCAGTCT | |
| | CTGAGCGTGACCTGTACAGTCAGCGGAGTGTCCCTGCC | |
| | TGATTACGGCGTGTCCTGGATCAGACAGCCTCCTCGGA | |
| | AAGGCCTGGAATGGCTGGGAGTGATCTGGGGCAGCGA | |
| | GACAACCTACTACAACAGCGCCCTGAAGTCCCGGCTG | |
| | ACCATCATCAAGGACAACTCCAAGAGCCAGGTGTTCCT | |
| | GAAGATGAACAGCCTGCAGACCGACGACACCGCCATC | |
| | TACTATTGCGCCAAGCACTACTACTACGGCGGCAGCTA | |
| | CGCCATGGATTATTGGGGCCAGGGCACCAGCGTGACC | |
| | GTGTCTAGCATCGAAGTGATGTACCCTCCACCTTACCT | |
| | GGACAACGAGAAGTCCAACGGCACCATCATCCACGTG | |
| | AAGGGCAAGCACCTGTGTCCTTCTCCACTGTTCCCCGG | |
| | ACCTAGCAAGCCTTTCTGGGTGCTCGTTGTTGTTGGCG | |
| | GCGTGCTGGCCTGTTATAGCCTGCTTGTGACCGTGGCC | |
| | TTCATCATCTTTTGGGTCCGAAGCAAGCGGAGCCGGCT | |
| | GCTGCACTCCGACTACATGAACATGACCCCTAGACGGC | |
| | CCGGACCAACCAGAAAGCACTACCAGCCTTACGCTCCT | |
| | CCTAGAGACTTCGCCGCCTACCGGTCCAGAGTGAAGTT | |
| | CAGCAGGAGCGCAGACGCCCCCGCGTACCAGCAGGGC | |
| | CAGAACCAGCTCTATAACGAGCTCAATCTAGGACGAA | |
| | GAGAGGAGTACGATGTTTTGGACAAGCGACGTGGCCG | |
| | GGACCCTGAGATGGGGGGAAAGCCGAGAAGGAAGAA | |
| | CCCTCAGGAAGGCCTGTACAATGAACTGCAGAAAGAT | |
| | AAGATGGCGGAGGCCTACAGTGAGATTGGGATGAAAG | |
| | GCGAGCGCCGGAGGGGCAAGGGGCACGATGGCCTTTA | |
| | CCAGGGACTCAGTACAGCCACCAAGGACACCTACGAC | |
| | GCCCTTCACATGCAGGCCCTGCCCCCTCGC | |
| A01_FMC63_ | ATGGCCAGATCTCCTGCTCAACTGCTGGGACTGCTGCT | 138 |
| CD28_CD28_ | GCTGTGGCTTAGCGGAGCCAGATGCGAGGTACAACTTT | |
| CD28_CD3Z | TGGAGTCAGGCGGTGGACTGGTACAACCGGGTGGTTC | |
| (P2014) | ATTGCGTTTGAGCTGCGCTGCCTCTGGTTTGACCTCTTA | |
| | TTCCTACGCGATGGGCTGGTATCGCCAAGCGCCGGGCA | |
| | AAGAACGCGAGTTTGTCAGCGCAATCAGCTCGGGTGG | |
| | TAGCGCGTACTACGCGGACTCGGTAAAAGGCCGTTTTA | |
| | CGATCAGTCGTGATAATTCCAAGAATACCTTGTACCTG | |
| | CAAATGAATAGCCTTCGCGCAGAAGACACAGCGGTGT | |
| | ATTATTGTGCCGTTGGACCGTACTACGGATTTAGAGCG | |
| | GTTACCGAAGCAGATTATTGGGGCCAGGGTACCCAGG | |
| | TGACGGTCTCGAGCGGCAGTACTTCTGGTAGCGGAAA | |
| | ACCCGGTAGCGGCGAGGGGTCAACTAAAGGAGACATC | |
| | CAGATGACCCAGACCACAAGCAGCCTGTCTGCCAGCCT | |
| | GGGCGATAGAGTGACCATCAGCTGTAGAGCCAGCCAG | |
| | GACATCAGCAAGTACCTGAACTGGTATCAGCAAAAGC | |
| | CCGACGGCACCGTGAAGCTGCTGATCTACCACACCAGC | |
| | AGACTGCACAGCGGCGTGCCAAGCAGATTTTCTGGCA | |
| | GCGGCTCTGGCACCGACTACAGCCTGACAATCAGCAA | |
| | CCTGGAACAAGAGGATATCGCTACCTACTTCTGCCAGC | |
| | AAGGCAACACCCTGCCTTACACCTTTGGCGGAGGCACC | |
| | AAGCTGGAAATCACCGGCTCTACAAGCGGCAGCGGCA | |
| | AACCTGGATCTGGCGAGGGATCTACCAAGGGCGAAGT | |
| | GAAACTGCAAGAGTCTGGCCCTGGACTGGTGGCCCCAT | |
| | CTCAGTCTCTGAGCGTGACCTGTACAGTCAGCGGAGTG | |
| | TCCCTGCCTGATTACGGCGTGTCCTGGATCAGACAGCC | |
| | TCCTCGGAAAGGCCTGGAATGGCTGGGAGTGATCTGG | |
| | GGCAGCGAGACAACCTACTACAACAGCGCCCTGAAGT | |
| | CCCGGCTGACCATCATCAAGGACAACTCCAAGAGCCA | |
| | GGTGTTCCTGAAGATGAACAGCCTGCAGACCGACGAC | |
| | ACCGCCATCTACTATTGCGCCAAGCACTACTACTACGG | |
| | CGGCAGCTACGCCATGGATTATTGGGGCCAGGGCACC | |
| | AGCGTGACCGTGTCTAGCATCGAAGTGATGTACCCTCC | |
| | ACCTTACCTGGACAACGAGAAGTCCAACGGCACCATC | |
| | ATCCACGTGAAGGGCAAGCACCTGTGTCCTTCTCCACT | |
| | GTTCCCCGGACCTAGCAAGCCTTTCTGGGTGCTCGTTG | |
| | TTGTTGGCGGCGTGCTGGCCTGTTATAGCCTGCTTGTG | |
| | ACCGTGGCCTTCATCATCTTTTGGGTCCGAAGCAAGCG | |
| | GAGCCGGCTGCTGCACTCCGACTACATGAACATGACCC | |
| | CTAGACGGCCCGGACCAACCAGAAAGCACTACCAGCC | |
| | TTACGCTCCTCCTAGAGACTTCGCCGCCTACCGGTCCA | |
| | GAGTGAAGTTCAGCAGATCCGCCGATGCTCCCGCCTAT | |
| | CAGCAGGGCCAAAACCAGCTGTACAACGAGCTGAACC | |
| | TGGGGAGAAGAGAAGAGTACGACGTGCTGGACAAGCG | |
| | GAGAGGCAGAGATCCTGAAATGGGCGGCAAGCCCAGA | |
| | CGGAAGAATCCTCAAGAGGGCCTGTATAATGAGCTGC | |
| | AGAAAGACAAGATGGCCGAGGCCTACAGCGAGATCGG | |
| | AATGAAGGGCGAGCGCAGAAGAGGCAAGGGACACGA | |
| | TGGACTGTACCAGGGCCTGAGCACCGCCACCAAGGAT | |
| | ACCTATGATGCCCTGCACATGCAGGCCCTGCCTCCAAG | |
| | A | |
| D04AMD11_E04_ | ATGGCCAGATCTCCTGCTCAACTGCTGGGACTGCTGCT | 139 |
| FMC63_CD28_ | GCTGTGGCTTAGCGGAGCCAGATGCCAGGTGCAGCTG | |
| CD28_CD28_ | GTTGAGTCTGGGGGAGGCCTTGTCCAGGCTGGGGGGTC | |
| CD3Z | CCTGAGACTCTCCTGTGCAGCGTCTGGAAGCGAATTCA | |
| (P2015) | CCGGTTATCCCATGGGCTGGTTTCGCCAGGCTCCAGGC | |
| | AAGGAAAGGGAGTTTGTCGCTGGCTCCGTAGGTATCG | |
| | GTGGTAGTACAAACTATGCAGACTCCGTGAAGGGCCG | |
| | ATTCACCATCTCCAGAGACAATGCGAAGAACACGGTCT | |
| | ATCTGCAAATGAACAGCCTGAAGCCAGAGGACACGGC | |
| | TGTGTATTACTGTGCGGCCGACAAAGACTACTACAAAC | |
| | CTTATAGTCGATATAGGACCGCTATCAGGTACGATACC | |
| | TGGGGCCAAGGGACCCAGGTCACCGTCTCGAGCGGCG | |
| | GTGGCGGATCAGAGGTACAACTTTTGGAGTCAGGCGG | |
| | TGGACTGGTACAACCGGGTGGTTCATTGCGTTTGAGCT | |
| | GCGCTGCCTCTGGTTTTACCAGCTCCTCCTACGTGATG | |
| | GGCTGGTATCGCCAAGCGCCGGGCAAAGAACGCGAGT | |
| | TTGTCAGCTCGATCAGCACCGGTGGTGATGCCTACTAC | |
| | GCGGACTCGGTAAAAGGCCGTTTTACGATCAGTCGTGA | |
| | TAATTCCAAGAATACCTTGTACCTGCAAATGAATAGCC | |
| | TTCGCGCAGAAGACACAGCGGTGTATTATTGTGCCGCT | |
| | GACGTTTGGTACTACCACGGCGGCGCGTACGATTATTG | |
| | GGGCCAGGGTACCCAGGTGACGGTCTCGAGCGGCAGT | |
| | ACTTCTGGTAGCGGAAAACCCGGTAGCGGCGAGGGGT | |
| | CAACTAAAGGAGACATCCAGATGACCCAGACCACAAG | |
| | CAGCCTGTCTGCCAGCCTGGGCGATAGAGTGACCATCA | |
| | GCTGTAGAGCCAGCCAGGACATCAGCAAGTACCTGAA | |
| | CTGGTATCAGCAAAAGCCCGACGGCACCGTGAAGCTG | |
| | CTGATCTACCACACCAGCAGACTGCACAGCGGCGTGCC | |
| | AAGCAGATTTTCTGGCAGCGGCTCTGGCACCGACTACA | |
| | GCCTGACAATCAGCAACCTGGAACAAGAGGATATCGC | |
| | TACCTACTTCTGCCAGCAAGGCAACACCCTGCCTTACA | |
| | CCTTTGGCGGAGGCACCAAGCTGGAAATCACCGGCTCT | |
| | ACAAGCGGCAGCGGCAAACCTGGATCTGGCGAGGGAT | |
| | CTACCAAGGGCGAAGTGAAACTGCAAGAGTCTGGCCC | |
| | TGGACTGGTGGCCCCATCTCAGTCTCTGAGCGTGACCT | |
| | GTACAGTCAGCGGAGTGTCCCTGCCTGATTACGGCGTG | |
| | TCCTGGATCAGACAGCCTCCTCGGAAAGGCCTGGAATG | |
| | GCTGGGAGTGATCTGGGGCAGCGAGACAACCTACTAC | |
| | AACAGCGCCCTGAAGTCCCGGCTGACCATCATCAAGG | |
| | ACAACTCCAAGAGCCAGGTGTTCCTGAAGATGAACAG | |
| | CCTGCAGACCGACGACACCGCCATCTACTATTGCGCCA | |
| | AGCACTACTACTACGGCGGCAGCTACGCCATGGATTAT | |
| | TGGGGCCAGGGCACCAGCGTGACCGTGTCTAGCATCG | |
| | AAGTGATGTACCCTCCACCTTACCTGGACAACGAGAAG | |
| | TCCAACGGCACCATCATCCACGTGAAGGGCAAGCACC | |
| | TGTGTCCTTCTCCACTGTTCCCCGGACCTAGCAAGCCTT | |
| | TCTGGGTGCTCGTTGTTGTTGGCGGCGTGCTGGCCTGTT | |
| | ATAGCCTGCTTGTGACCGTGGCCTTCATCATCTTTTGGG | |
| | TCCGAAGCAAGCGGAGCCGGCTGCTGCACTCCGACTA | |
| | CATGAACATGACCCCTAGACGGCCCGGACCAACCAGA | |
| | AAGCACTACCAGCCTTACGCTCCTCCTAGAGACTTCGC | |
| | CGCCTACCGGTCCAGAGTGAAGTTCAGCAGATCCGCCG | |
| | ATGCTCCCGCCTATCAGCAGGGCCAAAACCAGCTGTAC | |
| | AACGAGCTGAACCTGGGGAGAAGAGAAGAGTACGACG | |
| | TGCTGGACAAGCGGAGAGGCAGAGATCCTGAAATGGG | |
| | CGGCAAGCCCAGACGGAAGAATCCTCAAGAGGGCCTG | |
| | TATAATGAGCTGCAGAAAGACAAGATGGCCGAGGCCT | |
| | ACAGCGAGATCGGAATGAAGGGCGAGCGCAGAAGAG | |
| | GCAAGGGACACGATGGACTGTACCAGGGCCTGAGCAC | |
| | CGCCACCAAGGATACCTATGATGCCCTGCACATGCAGG | |
| | CCCTGCCTCCAAGA | |
| D04_E04_A01_ | ATGGCCAGATCTCCTGCTCAACTGCTGGGACTGCTGCT | |
| D04_FMC63_ | GCTGTGGCTTAGCGGAGCCAGATGCCAGGTGCAGCTG | |
| CD28_CD28_ | GTTGAGTCTGGGGGAGGCCTTGTCCAGGCTGGGGGGTC | |
| CD28_CD3Z | CCTGAGACTCTCCTGTGCAGCGTCTGGAAGCGAATTCA | |
| (P2016) | CCGGTTATCCCATGGGCTGGTTTCGCCAGGCTCCAGGC | |
| | AAGGAAAGGGAGTTTGTCGCTGGCTCCGTAGGTATCG | |
| | GTGGTAGTACAAACTATGCAGACTCCGTGAAGGGCCG | |
| | ATTCACCATCTCCAGAGACAATGCGAAGAACACGGTCT | |
| | ATCTGCAAATGAACAGCCTGAAGCCAGAGGACACGGC | 140 |
| | TGTGTATTACTGTGCGGCCGACAAAGACTACTACAAAC | |
| | CTTATAGTCGATATAGGACCGCTATCAGGTACGATACC | |
| | TGGGGCCAAGGGACCCAGGTCACCGTCTCGAGTGGCG | |
| | GTGGCGGATCAGAGGTACAACTTTTGGAGTCAGGCGG | |
| | TGGACTGGTACAACCGGGTGGTTCATTGCGTTTGAGCT | |
| | GCGCTGCCTCTGGTTTTACCAGCTCCTCCTACGTGATG | |
| | GGCTGGTATCGCCAAGCGCCGGGCAAAGAACGCGAGT | |
| | TTGTCAGCTCGATCAGCACCGGTGGTGATGCCTACTAC | |
| | GCGGACTCGGTAAAAGGCCGTTTTACGATCAGTCGTGA | |
| | TAATTCCAAGAATACCTTGTACCTGCAAATGAATAGCC | |
| | TTCGCGCAGAAGACACAGCGGTGTATTATTGTGCCGCT | |
| | GACGTTTGGTACTACCACGGCGGCGCGTACGATTATTG | |
| | GGGCCAGGGTACCCAGGTGACGGTCTCGAGCGGTGGC | |
| | GGTGGTTCTGAAGTCCAGCTGCTGGAAAGCGGTGGCG | |
| | GTCTGGTCCAGCCTGGCGGCACCCTGCGCCTGTCCTGT | |
| | GCCGCTAGCGGCCTGACCTGCTATAGCTATGCCATGGG | |
| | TTGGTACCGCCAGGCCCCTGGTAAGGAGCGCGAATTCG | |
| | TGTCCGCTATTTCCAGCGGCGGCTCCGCCTATTATGCT | |
| | GATAGCGTCAAGGGTCGCTTCACCATTTGCCGCGACAA | |
| | CAGCAAAAACACTCTGTATCTGCAGATGAACTCCCTGC | |
| | GCGCTGAGGATACCGCCGTCTACTACTGCGCTGTGGGC | |
| | CCTTATTATGGCTTCCGCGCTGTGACTGAGGCTGACTA | |
| | CTGGGGTCAGGGCACTCAGGTGACTGTGAGCAGCGGT | |
| | GGTGGCGGATCTCAGGTCCAGCTGGTGGAAAGCGGCG | |
| | GTGGTCTGGTGCAGGCTGGCGGTAGCCTGCGCCTGAGC | |
| | TGCGCTGCCAGCGGTTCCGAGTTTACTGGCTACCCTAT | |
| | GGGTTGGTTCCGCCAGGCCCCCGGTAAAGAGCGCGAA | |
| | TTCGTGGCCGGTAGCGTCGGCATTGGCGGCTCCACTAA | |
| | TTACGCTGATAGCGTCAAAGGTCGCTTTACTATTAGCC | |
| | GCGATAACGCCAAAAATACTGTGTACCTGCAGATGAA | |
| | TTCCCTGAAACCCGAAGATACCGCCGTCTACTATTGCG | |
| | CCGCTGATAAGGATTATTATAAGCCCTACTCCCGCTAC | |
| | CGCACTGCCATTCGCTATGACACTTGGGGTCAGGGCAC | |
| | TCAGGTGACTGTGAGCTCCGGCAGTACTTCTGGTAGCG | |
| | GAAAACCCGGTAGCGGCGAGGGGTCAACTAAAGGAGA | |
| | CATCCAGATGACCCAGACCACAAGCAGCCTGTCTGCCA | |
| | GCCTGGGCGATAGAGTGACCATCAGCTGTAGAGCCAG | |
| | CCAGGACATCAGCAAGTACCTGAACTGGTATCAGCAA | |
| | AAGCCCGACGGCACCGTGAAGCTGCTGATCTACCACA | |
| | CCAGCAGACTGCACAGCGGCGTGCCAAGCAGATTTTCT | |
| | GGCAGCGGCTCTGGCACCGACTACAGCCTGACAATCA | |
| | GCAACCTGGAACAAGAGGATATCGCTACCTACTTCTGC | |
| | CAGCAAGGCAACACCCTGCCTTACACCTTTGGCGGAGG | |
| | CACCAAGCTGGAAATCACCGGCTCTACAAGCGGCAGC | |
| | GGCAAACCTGGATCTGGCGAGGGATCTACCAAGGGCG | |
| | AAGTGAAACTGCAAGAGTCTGGCCCTGGACTGGTGGC | |
| | CCCATCTCAGTCTCTGAGCGTGACCTGTACAGTCAGCG | |
| | GAGTGTCCCTGCCTGATTACGGCGTGTCCTGGATCAGA | |
| | CAGCCTCCTCGGAAAGGCCTGGAATGGCTGGGAGTGA | |
| | TCTGGGGCAGCGAGACAACCTACTACAACAGCGCCCT | |
| | GAAGTCCCGGCTGACCATCATCAAGGACAACTCCAAG | |
| | AGCCAGGTGTTCCTGAAGATGAACAGCCTGCAGACCG | |
| | ACGACACCGCCATCTACTATTGCGCCAAGCACTACTAC | |
| | TACGGCGGCAGCTACGCCATGGATTATTGGGGCCAGG | |
| | GCACCAGCGTGACCGTGTCTAGCATCGAAGTGATGTAC | |
| | CCTCCACCTTACCTGGACAACGAGAAGTCCAACGGCAC | |
| | CATCATCCACGTGAAGGGCAAGCACCTGTGTCCTTCTC | |
| | CACTGTTCCCCGGACCTAGCAAGCCTTTCTGGGTGCTC | |
| | GTTGTTGTTGGCGGCGTGCTGGCCTGTTATAGCCTGCTT | |
| | GTGACCGTGGCCTTCATCATCTTTTGGGTCCGAAGCAA | |
| | GCGGAGCCGGCTGCTGCACTCCGACTACATGAACATG | |
| | ACCCCTAGACGGCCCGGACCAACCAGAAAGCACTACC | |
| | AGCCTTACGCTCCTCCTAGAGACTTCGCCGCCTACCGG | |
| | TCCAGAGTGAAGTTCAGCAGATCCGCCGATGCTCCCGC | |
| | CTATCAGCAGGGCCAAAACCAGCTGTACAACGAGCTG | |
| | AACCTGGGGAGAAGAGAAGAGTACGACGTGCTGGACA | |
| | AGCGGAGAGGCAGAGATCCTGAAATGGGCGGCAAGCC | |
| | CAGACGGAAGAATCCTCAAGAGGGCCTGTATAATGAG | |
| | CTGCAGAAAGACAAGATGGCCGAGGCCTACAGCGAGA | |
| | TCGGAATGAAGGGCGAGCGCAGAAGAGGCAAGGGAC | |
| | ACGATGGACTGTACCAGGGCCTGAGCACCGCCACCAA | |
| | GGATACCTATGATGCCCTGCACATGCAGGCCCTGCCTC | |
| | CAAGA | |
| FMC63LC_A01_ | ATGGCCAGATCTCCTGCTCAACTGCTGGGACTGCTGCT | 141 |
| A01_FMC63HC_ | GCTGTGGCTTAGCGGAGCCAGATGCGACATCCAGATG | |
| CD28_CD28_ | ACCCAGACCACAAGCAGCCTGTCTGCCAGCCTGGGCG | |
| CD28_CD3Z | ATAGAGTGACCATCAGCTGTAGAGCCAGCCAGGACAT | |
| (P2193) | CAGCAAGTACCTGAACTGGTATCAGCAAAAGCCCGAC | |
| | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTGACCTCTT | |
| | ATTCCTACGCGATGGGCTGGTATCGCCAAGCGCCGGGC | |
| | AAAGAACGCGAGTTTGTCAGCGCAATCAGCTCGGGTG | |
| | GTAGCGCGTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGTTGGACCGTACTACGGATTTAGAGC | |
| | GGTTACCGAAGCAGATTATTGGGGCCAGGGTACCCAG | |
| | GTGACGGTCTCGAGCGGCGGTGGCGGATCAGAAGTCC | |
| | AGCTGCTGGAAAGCGGTGGCGGTCTGGTCCAGCCTGG | |
| | CGGCACCCTGCGCCTGTCCTGTGCCGCTAGCGGCCTGA | |
| | CCTGCTATAGCTATGCCATGGGTTGGTACCGCCAGGCC | |
| | CCTGGTAAGGAGCGCGAATTCGTGTCCGCTATTTCCAG | |
| | CGGCGGCTCCGCCTATTATGCTGATAGCGTCAAGGGTC | |
| | GCTTCACCATTTGCCGCGACAACAGCAAAAACACTCTG | |
| | TATCTGCAGATGAACTCCCTGCGCGCTGAGGATACCGC | |
| | CGTCTACTACTGCGCTGTGGGCCCTTATTATGGCTTCCG | |
| | CGCTGTGACTGAGGCTGACTACTGGGGTCAGGGCACTC | |
| | AGGTGACTGTGAGCAGCGGCAGTACTTCTGGTAGCGG | |
| | AAAACCCGGTAGCGGCGAGGGGTCAACTAAAGGAGAA | |
| | GTGAAACTGCAAGAGTCTGGCCCTGGACTGGTGGCCCC | |
| | ATCTCAGTCTCTGAGCGTGACCTGTACAGTCAGCGGAG | |
| | TGTCCCTGCCTGATTACGGCGTGTCCTGGATCAGACAG | |
| | CCTCCTCGGAAAGGCCTGGAATGGCTGGGAGTGATCTG | |
| | GGGCAGCGAGACAACCTACTACAACAGCGCCCTGAAG | |
| | TCCCGGCTGACCATCATCAAGGACAACTCCAAGAGCC | |
| | AGGTGTTCCTGAAGATGAACAGCCTGCAGACCGACGA | |
| | CACCGCCATCTACTATTGCGCCAAGCACTACTACTACG | |
| | GCGGCAGCTACGCCATGGATTATTGGGGCCAGGGCAC | |
| | CAGCGTGACCGTGTCTAGCATCGAAGTGATGTACCCTC | |
| | CACCTTACCTGGACAACGAGAAGTCCAACGGCACCAT | |
| | CATCCACGTGAAGGGCAAGCACCTGTGTCCTTCTCCAC | |
| | TGTTCCCCGGACCTAGCAAGCCTTTCTGGGTGCTCGTT | |
| | GTTGTTGGCGGCGTGCTGGCCTGTTATAGCCTGCTTGT | |
| | GACCGTGGCCTTCATCATCTTTTGGGTCCGAAGCAAGC | |
| | GGAGCCGGCTGCTGCACTCCGACTACATGAACATGACC | |
| | CCTAGACGGCCCGGACCAACCAGAAAGCACTACCAGC | |
| | CTTACGCTCCTCCTAGAGACTTCGCCGCCTACCGGTCC | |
| | AGAGTGAAGTTCAGCAGATCCGCCGATGCTCCCGCCTA | |
| | TCAGCAGGGCCAAAACCAGCTGTACAACGAGCTGAAC | |
| | CTGGGGAGAAGAGAAGAGTACGACGTGCTGGACAAGC | |
| | GGAGAGGCAGAGATCCTGAAATGGGCGGCAAGCCCAG | |
| | ACGGAAGAATCCTCAAGAGGGCCTGTATAATGAGCTG | |
| | CAGAAAGACAAGATGGCCGAGGCCTACAGCGAGATCG | |
| | GAATGAAGGGCGAGCGCAGAAGAGGCAAGGGACACG | |
| | ATGGACTGTACCAGGGCCTGAGCACCGCCACCAAGGA | |
| | TACCTATGATGCCCTGCACATGCAGGCCCTGCCTCCAA | |
| | GA | |
| FMC63LC_D0_ | ATGGCCAGATCTCCTGCTCAACTGCTGGGACTGCTGCT | 142 |
| 4AMD11_A01_ | GCTGTGGCTTAGCGGAGCCAGATGCGACATCCAGATG | |
| FMC63HC_ | ACCCAGACCACAAGCAGCCTGTCTGCCAGCCTGGGCG | |
| CD28_CD28_ | ATAGAGTGACCATCAGCTGTAGAGCCAGCCAGGACAT | |
| CD28_CD3Z | CAGCAAGTACCTGAACTGGTATCAGCAAAAGCCCGAC | |
| (P2191) | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCCAGGTGCAGCT | |
| | GGTTGAGTCTGGGGGAGGCCTTGTCCAGGCTGGGGGG | |
| | TCCCTGAGACTCTCCTGTGCAGCGTCTGGAAGCGAATT | |
| | CACCGGTTATCCCATGGGCTGGTTTCGCCAGGCTCCAG | |
| | GCAAGGAAAGGGAGTTTGTCGCTGGCTCCGTAGGTATC | |
| | GGTGGTAGTACAAACTATGCAGACTCCGTGAAGGGCC | |
| | GATTCACCATCTCCAGAGACAATGCGAAGAACACGGT | |
| | CTATCTGCAAATGAACAGCCTGAAGCCAGAGGACACG | |
| | GCTGTGTATTACTGTGCGGCCGACAAAGACTACTACAA | |
| | ACCTTATAGTCGATATAGGACCGCTATCAGGTACGATA | |
| | CCTGGGGCCAAGGGACCCAGGTCACCGTCTCGAGCGG | |
| | CGGTGGCGGATCAGAAGTCCAGCTGCTGGAAAGCGGT | |
| | GGCGGTCTGGTCCAGCCTGGCGGCACCCTGCGCCTGTC | |
| | CTGTGCCGCTAGCGGCCTGACCTGCTATAGCTATGCCA | |
| | TGGGTTGGTACCGCCAGGCCCCTGGTAAGGAGCGCGA | |
| | ATTCGTGTCCGCTATTTCCAGCGGCGGCTCCGCCTATT | |
| | ATGCTGATAGCGTCAAGGGTCGCTTCACCATTTGCCGC | |
| | GACAACAGCAAAAACACTCTGTATCTGCAGATGAACT | |
| | CCCTGCGCGCTGAGGATACCGCCGTCTACTACTGCGCT | |
| | GTGGGCCCTTATTATGGCTTCCGCGCTGTGACTGAGGC | |
| | TGACTACTGGGGTCAGGGCACTCAGGTGACTGTGAGC | |
| | AGCGGCAGTACTTCTGGTAGCGGAAAACCCGGTAGCG | |
| | GCGAGGGGTCAACTAAAGGAGAAGTGAAACTGCAAGA | |
| | GTCTGGCCCTGGACTGGTGGCCCCATCTCAGTCTCTGA | |
| | GCGTGACCTGTACAGTCAGCGGAGTGTCCCTGCCTGAT | |
| | TACGGCGTGTCCTGGATCAGACAGCCTCCTCGGAAAGG | |
| | CCTGGAATGGCTGGGAGTGATCTGGGGCAGCGAGACA | |
| | ACCTACTACAACAGCGCCCTGAAGTCCCGGCTGACCAT | |
| | CATCAAGGACAACTCCAAGAGCCAGGTGTTCCTGAAG | |
| | ATGAACAGCCTGCAGACCGACGACACCGCCATCTACT | |
| | ATTGCGCCAAGCACTACTACTACGGCGGCAGCTACGCC | |
| | ATGGATTATTGGGGCCAGGGCACCAGCGTGACCGTGTC | |
| | TAGCATCGAAGTGATGTACCCTCCACCTTACCTGGACA | |
| | ACGAGAAGTCCAACGGCACCATCATCCACGTGAAGGG | |
| | CAAGCACCTGTGTCCTTCTCCACTGTTCCCCGGACCTA | |
| | GCAAGCCTTTCTGGGTGCTCGTTGTTGTTGGCGGCGTG | |
| | CTGGCCTGTTATAGCCTGCTTGTGACCGTGGCCTTCATC | |
| | ATCTTTTGGGTCCGAAGCAAGCGGAGCCGGCTGCTGCA | |
| | CTCCGACTACATGAACATGACCCCTAGACGGCCCGGAC | |
| | CAACCAGAAAGCACTACCAGCCTTACGCTCCTCCTAGA | |
| | GACTTCGCCGCCTACCGGTCCAGAGTGAAGTTCAGCAG | |
| | ATCCGCCGATGCTCCCGCCTATCAGCAGGGCCAAAACC | |
| | AGCTGTACAACGAGCTGAACCTGGGGAGAAGAGAAGA | |
| | GTACGACGTGCTGGACAAGCGGAGAGGCAGAGATCCT | |
| | GAAATGGGCGGCAAGCCCAGACGGAAGAATCCTCAAG | |
| | AGGGCCTGTATAATGAGCTGCAGAAAGACAAGATGGC | |
| | CGAGGCCTACAGCGAGATCGGAATGAAGGGCGAGCGC | |
| | AGAAGAGGCAAGGGACACGATGGACTGTACCAGGGCC | |
| | TGAGCACCGCCACCAAGGATACCTATGATGCCCTGCAC | |
| | ATGCAGGCCCTGCCTCCAAGA | |
| FMC63LC_A01_ | ATGGCCAGATCTCCTGCTCAACTGCTGGGACTGCTGCT | 143 |
| D04AMD11_ | GCTGTGGCTTAGCGGAGCCAGATGCGACATCCAGATG | |
| A01_FMC63HC_ | ACCCAGACCACAAGCAGCCTGTCTGCCAGCCTGGGCG | |
| CD28_CD28_ | ATAGAGTGACCATCAGCTGTAGAGCCAGCCAGGACAT | |
| CD28_CD3Z | CAGCAAGTACCTGAACTGGTATCAGCAAAAGCCCGAC | |
| (P2195) | GGCACCGTGAAGCTGCTGATCTACCACACCAGCAGACT | |
| | GCACAGCGGCGTGCCAAGCAGATTTTCTGGCAGCGGCT | |
| | CTGGCACCGACTACAGCCTGACAATCAGCAACCTGGA | |
| | ACAAGAGGATATCGCTACCTACTTCTGCCAGCAAGGCA | |
| | ACACCCTGCCTTACACCTTTGGCGGAGGCACCAAGCTG | |
| | GAAATCACCGGCTCTACAAGCGGCAGCGGCAAACCTG | |
| | GATCTGGCGAGGGATCTACCAAGGGCGAGGTACAACT | |
| | TTTGGAGTCAGGCGGTGGACTGGTACAACCGGGTGGTT | |
| | CATTGCGTTTGAGCTGCGCTGCCTCTGGTTTGACCTCTT | |
| | ATTCCTACGCGATGGGCTGGTATCGCCAAGCGCCGGGC | |
| | AAAGAACGCGAGTTTGTCAGCGCAATCAGCTCGGGTG | |
| | GTAGCGCGTACTACGCGGACTCGGTAAAAGGCCGTTTT | |
| | ACGATCAGTCGTGATAATTCCAAGAATACCTTGTACCT | |
| | GCAAATGAATAGCCTTCGCGCAGAAGACACAGCGGTG | |
| | TATTATTGTGCCGTTGGACCGTACTACGGATTTAGAGC | |
| | GGTTACCGAAGCAGATTATTGGGGCCAGGGTACCCAG | |
| | GTGACGGTCTCGAGCGGCGGTGGCGGATCACAGGTGC | |
| | AGCTGGTTGAGTCTGGGGGAGGCCTTGTCCAGGCTGGG | |
| | GGGTCCCTGAGACTCTCCTGTGCAGCGTCTGGAAGCGA | |
| | ATTCACCGGTTATCCCATGGGCTGGTTTCGCCAGGCTC | |
| | CAGGCAAGGAAAGGGAGTTTGTCGCTGGCTCCGTAGG | |
| | TATCGGTGGTAGTACAAACTATGCAGACTCCGTGAAGG | |
| | GCCGATTCACCATCTCCAGAGACAATGCGAAGAACAC | |
| | GGTCTATCTGCAAATGAACAGCCTGAAGCCAGAGGAC | |
| | ACGGCTGTGTATTACTGTGCGGCCGACAAAGACTACTA | |
| | CAAACCTTATAGTCGATATAGGACCGCTATCAGGTACG | |
| | ATACCTGGGGCCAAGGGACCCAGGTCACCGTCTCGAG | |
| | CGGTGGCGGTGGTTCTGAAGTCCAGCTGCTGGAAAGC | |
| | GGTGGCGGTCTGGTCCAGCCTGGCGGCACCCTGCGCCT | |
| | GTCCTGTGCCGCTAGCGGCCTGACCTGCTATAGCTATG | |
| | CCATGGGTTGGTACCGCCAGGCCCCTGGTAAGGAGCG | |
| | CGAATTCGTGTCCGCTATTTCCAGCGGCGGCTCCGCCT | |
| | ATTATGCTGATAGCGTCAAGGGTCGCTTCACCATTTGC | |
| | CGCGACAACAGCAAAAACACTCTGTATCTGCAGATGA | |
| | ACTCCCTGCGCGCTGAGGATACCGCCGTCTACTACTGC | |
| | GCTGTGGGCCCTTATTATGGCTTCCGCGCTGTGACTGA | |
| | GGCTGACTACTGGGGTCAGGGCACTCAGGTGACTGTG | |
| | AGCAGCGGCAGTACTTCTGGTAGCGGAAAACCCGGTA | |
| | GCGGCGAGGGGTCAACTAAAGGAGAAGTGAAACTGCA | |
| | AGAGTCTGGCCCTGGACTGGTGGCCCCATCTCAGTCTC | |
| | TGAGCGTGACCTGTACAGTCAGCGGAGTGTCCCTGCCT | |
| | GATTACGGCGTGTCCTGGATCAGACAGCCTCCTCGGAA | |
| | AGGCCTGGAATGGCTGGGAGTGATCTGGGGCAGCGAG | |
| | ACAACCTACTACAACAGCGCCCTGAAGTCCCGGCTGAC | |
| | CATCATCAAGGACAACTCCAAGAGCCAGGTGTTCCTGA | |
| | AGATGAACAGCCTGCAGACCGACGACACCGCCATCTA | |
| | CTATTGCGCCAAGCACTACTACTACGGCGGCAGCTACG | |
| | CCATGGATTATTGGGGCCAGGGCACCAGCGTGACCGT | |
| | GTCTAGCATCGAAGTGATGTACCCTCCACCTTACCTGG | |
| | ACAACGAGAAGTCCAACGGCACCATCATCCACGTGAA | |
| | GGGCAAGCACCTGTGTCCTTCTCCACTGTTCCCCGGAC | |
| | CTAGCAAGCCTTTCTGGGTGCTCGTTGTTGTTGGCGGC | |
| | GTGCTGGCCTGTTATAGCCTGCTTGTGACCGTGGCCTT | |
| | CATCATCTTTTGGGTCCGAAGCAAGCGGAGCCGGCTGC | |
| | TGCACTCCGACTACATGAACATGACCCCTAGACGGCCC | |
| | GGACCAACCAGAAAGCACTACCAGCCTTACGCTCCTCC | |
| | TAGAGACTTCGCCGCCTACCGGTCCAGAGTGAAGTTCA | |
| | GCAGATCCGCCGATGCTCCCGCCTATCAGCAGGGCCAA | |
| | AACCAGCTGTACAACGAGCTGAACCTGGGGAGAAGAG | |
| | AAGAGTACGACGTGCTGGACAAGCGGAGAGGCAGAGA | |
| | TCCTGAAATGGGCGGCAAGCCCAGACGGAAGAATCCT | |
| | CAAGAGGGCCTGTATAATGAGCTGCAGAAAGACAAGA | |
| | TGGCCGAGGCCTACAGCGAGATCGGAATGAAGGGCGA | |
| | GCGCAGAAGAGGCAAGGGACACGATGGACTGTACCAG | |
| | GGCCTGAGCACCGCCACCAAGGATACCTATGATGCCCT | |
| | GCACATGCAGGCCCTGCCTCCAAGA | |

In some embodiments, the antigen-binding domain of the second polypeptide binds to an antigen. The antigen-binding domain of the second polypeptide may bind to more than one antigen or more than one epitope in an antigen. For example, the antigen-binding domain of the second polypeptide may bind to two, three, four, five, six, seven, eight or more antigens. As another example, the antigen-binding domain of the second polypeptide may bind to two, three, four, five, six, seven, eight or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79, BCMA, GPRCSD, SLAM F7, CD33, CLL1, CD123, and CD70. Non-limiting examples of tumor antigens associated with bladder cancer include Nectin-4 and SLITRK6.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAMS), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, 5100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 2, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In one embodiment, the anti-CD19 scFv comprises a light chain variable region (VL) comprising the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In one embodiment, the anti-CD19 scFv comprises the amino acid sequence set forth in SEQ ID NO: 7, or a variant thereof having at least 50, at least 55, at least 60, at least 65, at least at least 75, at least 80, at least 85, at least 90, at least 95, at least 96, at least 97, at least 98 or at least 99%, sequence identity with SEQ ID NO: 7.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, fillaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic trasnlation elogation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB7B), U1RNP, A2/B1 hnRNP, Ro (SSA), and La (SSB) antigens.

In various embodiments, the scFv fragment used in the CAR of the present disclosure may include a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G₄S)₃ linker. In one embodiment, the (G₄S)₃ linker comprises the amino acid sequence set forth in SEQ ID NO: or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences may include portions of immunoglobulin hinge area, CL or CH1 derived from any immunoglobulin heavy or light chain isotype. Exemplary linkers that may be used include any of SEQ ID NOs: 26-56 in Table 1. Additional linkers are described for example in Int. Pat. Publ. No. WO2019/060695, incorporated by reference herein in its entirety.

### III. Artificial Cell Death Polypeptide

According to embodiments of the application, an iPSC cell or a derivative cell thereof comprises a second exogenous polynucleotide encoding an artificial cell death polypeptide.

As used herein, the term “artificial cell death polypeptide” refers to an engineered protein designed to prevent potential toxicity or otherwise adverse effects of a cell therapy. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g. an antibody, that when activated, triggers apoptosis and/or cell death of a therapeutic cell.

In certain embodiments, an artificial cell death polypeptide comprises an inactivated cell surface receptor that comprises an epitope specifically recognized by an antibody, particularly a monoclonal antibody, which is also referred to herein as a monoclonal antibody-specific epitope. When expressed by iPSCs or derivative cells thereof, the inactivated cell surface receptor is signaling inactive or significantly impaired, but can still be specifically recognized by an antibody. The specific binding of the antibody to the inactivated cell surface receptor enables the elimination of the iPSCs or derivative cells thereof by ADCC and/or ADCP mechanisms, as well as, direct killing with antibody drug conjugates with toxins or radionuclides.

In certain embodiments, the inactivated cell surface receptor comprises an epitope that is selected from epitopes specifically recognized by an antibody, including but not limited to, ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, polatuzumab vedotin, ranibizumab, tocilizumab, trastuzumab, vedolizumab, adalimumab, belimumab, canakinumab, denosumab, golimumab, ipilimumab, ofatumumab, panitumumab, or ustekinumab.

Epidermal growth factor receptor, also known as EGFR, ErbB1 and HER1, is a cell-surface receptor for members of the epidermal growth factor family of extracellular ligands. As used herein, “truncated EGFR,” “tEGFR,” “short EGFR” or “sEGFR” refers to an inactive EGFR variant that lacks the EGF-binding domains and the intracellular signaling domains of the EGFR. An exemplary tEGFR variant contains residues 322-333 of domain 2, all of domains 3 and 4 and the transmembrane domain of the native EGFR sequence containing the cetuximab binding epitope. Expression of the tEGFR variant on the cell surface enables cell elimination by an antibody that specifically binds to the tEGFR, such as cetuximab (Erbitux®), as needed. Due to the absence of the EGF-binding domains and intracellular signaling domains, tEGFR is inactive when expressed by iPSCs or derivative cell thereof.

An exemplary inactivated cell surface receptor of the application comprises a tEGFR variant. In certain embodiments, expression of the inactivated cell surface receptor in an engineered immune cell expressing a chimeric antigen receptor (CAR) induces cell suicide of the engineered immune cell when the cell is contacted with an anti-EGFR antibody. Methods of using inactivated cell surface receptors are described in WO2019/070856, WO2019/023396, WO2018/058002, the disclosure of which is incorporated herein by reference. For example, a subject who has previously received an engineered immune cell of the present disclosure that comprises a heterologous polynucleotide encoding an inactivated cell surface receptor comprising a tEGFR variant can be administered an anti-EGFR antibody in an amount effective to ablate in the subject the previously administered engineered immune cell.

In certain embodiments, the anti-EGFR antibody is cetuximab, matuzumab, necitumumab or panitumumab, preferably the anti-EGFR antibody is cetuximab.

In certain embodiments, the tEGFR variant comprises or consists of an amino acid sequence at least 90%, such as at least 90%, 91%, 92%, 93%, 94%, 95%, 96%, 97%, 98%, 99% or 100%, identical to SEQ ID NO: 71, preferably the amino acid sequence of SEQ ID NO: 71.

In some embodiments, the inactivated cell surface receptor comprises one or more epitopes of CD79b, such as an epitope specifically recognized by polatuzumab vedotin. In certain embodiments, the CD79b epitope comprises or consists of an amino acid sequence at least 90%, such as at least 90%, 91%, 92%, 93%, 94%, 95%, 96%, 97%, 98%, 99% or 100%, identical to SEQ ID NO: 78, preferably the amino acid sequence of SEQ ID NO: 78.

In some embodiments, the inactivated cell surface receptor comprises one or more epitopes of CD20, such as an epitope specifically recognized by rituximab. In certain embodiments, the CD20 epitope comprises or consists of an amino acid sequence at least 90%, such as at least 90%, 91%, 92%, 93%, 94%, 95%, 96%, 97%, 98%, 99% or 100%, identical to SEQ ID NO: 80, preferably the amino acid sequence of SEQ ID NO: 80.

In some embodiments, the inactivated cell surface receptor comprises one or more epitopes of Her 2 receptor or ErbB, such as an epitope specifically recognized by trastuzumab. In certain embodiments, the monoclonal antibody-specific epitope comprises or consists of an amino acid sequence at least 90%, such as at least 90%, 91%, 92%, 93%, 94%, 95%, 96%, 97%, 98%, 99% or 100%, identical to SEQ ID NO: 82, preferably the amino acid sequence of SEQ ID NO: 82.

In some embodiments the inactivated cell surface receptor further comprises a cytokine, such as interleukin-15 or interleukin-2.

As used herein “Interleukin-15” or “IL-15” refers to a cytokine that regulates T and NK cell activation and proliferation, or a functional portion thereof. A “functional portion” (“biologically active portion”) of a cytokine refers to a portion of the cytokine that retains one or more functions of full length or mature cytokine. Such functions for IL-15 include the promotion of NK cell survival, regulation of NK cell and T cell activation and proliferation as well as the support of NK cell development from hematopoietic stem cells. As will be appreciated by those of skill in the art, the sequence of a variety of IL-15 molecules are known in the art. In certain embodiments, the IL-15 is a wild-type IL-15. In certain embodiments, the IL-15 is a human IL-15. In certain embodiments, the IL-15 comprises an amino acid sequence at least 90%, such as at least 90%, 91%, 92%, 93%, 94%, 95%, 96%, 97%, 98%, 99% or 100%, identical to SEQ ID NO: 72, preferably the amino acid sequence of SEQ ID NO: 72.

As used herein “Interleukin-2” refers to a cytokine that regulates T and NK cell activation and proliferation, or a functional portion thereof. In certain embodiments, the IL-2 is a wild-type IL-2. In certain embodiments, the IL-2 is a human IL-2. In certain embodiments, the IL-2 comprises an amino acid sequence at least 90%, such as at least 90%, 91%, 92%, 93%, 94%, 95%, 96%, 97%, 98%, 99% or 100%, identical to SEQ ID NO: 76, preferably the amino acid sequence of SEQ ID NO: 76.

In certain embodiments, an inactivated cell surface receptor comprises a monoclonal antibody-specific epitope operably linked to a cytokine, preferably by an autoprotease peptide sequence. Examples of the autoprotease peptide include, but are not limited to, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In one embodiment, the autoprotease peptide is an autoprotease peptide of porcine tesehovirus-1 2A (P2A). In certain embodiments, the autoprotease peptide comprises an amino acid sequence at least 90%, such as at least 90%, 91%, 92%, 93%, 94%, 95%, 96%, 97%, 98%, 99% or 100%, identical to SEQ ID NO: 73, preferably the amino acid sequence of SEQ ID NO: 73.

In certain embodiments, an inactivated cell surface receptor comprises a truncated epithelial growth factor (tEGFR) variant operably linked to an interleukin-15 (IL-15) or IL-2 by an autoprotease peptide sequence. In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 74, preferably the amino acid sequence of SEQ ID NO: 74.

In some embodiments, an inactivated cell surface receptor further comprises a signal sequence. In certain embodiments, the signal sequence comprises an amino acid sequence at least 90%, such as at least 90%, 91%, 92%, 93%, 94%, 95%, 96%, 97%, 98%, 99% or 100%, identical to SEQ ID NO: 77, preferably the amino acid sequence of SEQ ID NO: 77.

In some embodiments, an inactivated cell surface receptor further comprises a hinge domain. In some embodiments, the hinge domain is derived from CD8. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21.

In certain embodiments, an inactivated cell surface receptor further comprises a transmembrane domain. In some embodiments, the transmembrane domain is derived from CD8. In one embodiment, the CD8 transmembrane domain comprises the amino acid sequence set forth in SEQ ID NO: 23, or a variant thereof having at least 50, at least at least 60, at least 65, at least 70, at least 75, at least 80, at least 85, at least 90, at least 95, at least 96, at least 97, at least 98 or at least 99%, sequence identity with SEQ ID NO: 23.

In certain embodiment, an inactivated cell surface receptor comprises one or more epitopes specifically recognized by an antibody in its extracellular domain, a transmembrane region and a cytoplasmic domain. In some embodiments, the inactivated cell surface receptor further comprises a hinge region between the epitope(s) and the transmembrane region. In some embodiments, the inactivated cell surface receptor comprises more than one epitopes specifically recognized by an antibody, the epitopes can have the same or different amino acid sequences, and the epitopes can be linked together via a peptide linker, such as a flexible peptide linker have the sequence of (GGGGS)n, wherein n is an integer of 1-8 (SEQ ID NO: 25). In some embodiments, the inactivated cell surface receptor further comprises a cytokine, such as an IL-15 or IL-2. In certain embodiments, the cytokine is in the cytoplasmic domain of the inactivated cell surface receptor. Preferably, the cytokine is operably linked to the epitope(s) specifically recognized by an antibody, directly or indirectly, via an autoprotease peptide sequence, such as those described herein. In some embodiments, the cytokine is indirectly linked to the epitope(s) by connecting to the transmembrane region via the autoprotease peptide sequence.

Non-limiting exemplary inactivated cell surface receptor regions and sequences are provided in Table 2.

TABLE 2

| | | |
|---|---|---|
| | | SEQ ID |
| Regions | Sequence | NO |

tEGFR-IL15:

| | | |
|---|---|---|
| tEGFR | MRPSGTAGAALLALLAALCPASRAGVRKCKKCEGPCRK | 71 |
| | VCNGIGIGEFKDSLSINATNIKHFKNCTSISGDLHILPVAF | |
| | RGDSFTHTPPLDPQELDILKTVKEITGFLLIQAWPENRTD | |
| | LHAFENLEIIRGRTKQHGQFSLAVVSLNITSLGLRSLKEIS | |
| | DGDVIISGNKNLCYANTINWKKLFGTSGQKTKIISNRGE | |
| | NSCKATGQVCHALCSPEGCWGPEPRDCVSCRNVSRGRE | |
| | CVDKCNLLEGEPREFVENSECIQCHPECLPQAMNITCTG | |
| | RGPDNCIQCAHYIDGPHCVKTCPAGVMGENNTLVWKY | |
| | ADAGHVCHLCHPNCTYGCTGPGLEGCPTNGPKIPSIATG | |
| | MVGALLLLLVVALGIGLFM | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

CD79b-IL15:

| | | |
|---|---|---|
| Signal | MEFGLSWVFLVALFRGVQC | 77 |
| Sequence | | |
| CD79b | ARSEDRYRNPKGSACSRIWQS | 78 |
| epitope | | |
| CD8 (AA | TTTPAPRPPTPAPTIASQPLSLRPEACRPAAGGAVHTRGL | 21 |
| 136-182) | DFACD | |
| CD8 (AA | IYIWAPLAGTCGVLLLSLVIT | 23 |
| 183-203) | | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

CD20 mimitope-IL15:

| | | |
|---|---|---|
| Signal | MEFGLSWVFLVALFRGVQC | 77 |
| Sequence | | |
| CD20 | ACPYANPSLC | 80 |
| mimitope | | |
| Linker | GGGSGGGS | 27 |
| CD8 (AA | TTTPAPRPPTPAPTIASQPLSLRPEACRPAAGGAVHTRGL | 21 |
| 136-182) | DFACD | |
| CD8 (AA | IYIWAPLAGTCGVLLLSLVIT | 23 |
| 183-203) | | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

ErbB epitope-IL15:

| | | |
|---|---|---|
| Signal | MEFGLSWVFLVALFRGVQC | 77 |
| Sequence | | |
| ErbB | EGLACHQLCARGHCWGPGPTQCVNCSQFLRGQECVEE | 82 |
| epitope | CRVLQGLPREYVNARHCLPCHPECQPQNGSVTCFGPEA | |
| | DQCVACAHYKDPPFCVARCPSGVKPDLSYMPIWKFPDE | |
| | EGACQPCPINCTHSCVDLDDKGCPAEQRASPLTSIISAVV | |
| | GILLVVVLGVVFGILIGGGGSGG | |
| P2A | ATNFSLLKQAGDVEENPGP | 73 |
| IL-15 | MRISKPHLRSISIQCYLCLLLNSHFLTEAGIHVFILGCFSA | 72 |
| | GLPKTEANWVNVISDLKKIEDLIQSMHIDATLYTESDVH | |
| | PSCKVTAMKCFLLELQVISLESGDASIHDTVENLIILANN | |
| | SLSSNGNVTESGCKECEELEEKNIKEFLQSFVHIVQMFIN | |
| | TS | |

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 79, preferably the amino acid sequence of SEQ ID NO: 79.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 81, preferably the amino acid sequence of SEQ ID NO: 81.

In a particular embodiment, the inactivated cell surface receptor comprises an amino acid sequence at least 90%, such as at least 90%, 91%, 92%, 93%, 94%, 95%, 96%, 97%, 98%, 99% or 100%, identical to SEQ ID NO: 83, preferably the amino acid sequence of SEQ ID NO: 83.

### IV. HLA Expression

In certain embodiments, an iPSC or derivative cell thereof of the application can be further modified by introducing a third exogenous polynucleotide encoding one or more proteins related to immune evasion, such as non-classical HLA class I proteins (e.g., HLA-E and HLA-G). In particular, disruption of the B2M gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol. 2017 August; 35(8): 765-772).

In certain embodiments, the iPSC or derivative cell thereof comprises a third exogenous polypeptide encoding at least one of a human leukocyte antigen E (HLA-E) and human leukocyte antigen G (HLA-G). In a particular embodiment, the HLA-E comprises an amino acid sequence at least 90%, such as at least 90%, 91%, 92%, 93%, 94%, 95%, 96%, 97%, 98%, 99% or 100%, identical to SEQ ID NO: 65, preferably the amino acid sequence of SEQ ID NO: 65. In a particular embodiment, the HLA-G comprises an amino acid sequence at least 90%, such as at least 90%, 91%, 92%, 93%, 94%, 95%, 96%, 97%, 98%, 99% or 100%, identical to SEQ ID NO: 68, preferably SEQ ID NO: 68.

In certain embodiments, the third exogenous polynucleotide encodes a polypeptide comprising a signal peptide operably linked to a mature B2M protein that is fused to an HLA-E via a linker. In a particular embodiment, the third exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 66.

In other embodiments, the third exogenous polynucleotide encodes a polypeptide comprising a signal peptide operably linked to a mature B2M protein that is fused to an HLA-G via a linker. In a particular embodiment, the third exogenous polypeptide comprises an amino acid sequence at least sequence at least 90%, such as at least 90%, 91%, 92%, 93%, 94%, 95%, 96%, 97%, 98%, 99% or 100%, identical to SEQ ID NO: 69.

### V. Other Optional Genome Edits

In one embodiment of the above described cell, the genomic editing at one or more selected sites may comprise insertions of one or more exogenous polynucleotides encoding other additional artificial cell death polypeptides, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof.

In some embodiments, the exogenous polynucleotides for insertion are operatively linked to (1) one or more exogenous promoters comprising CMV, EF1a, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, Hll, beta-2 microglobulin, GAPDH, TCR or RUNX1, or other locus meeting the criteria of a genome safe harbor. In some embodiments, the genome-engineered iPSCs generated using the above method comprise one or more different exogenous polynucleotides encoding proteins comprising caspase, thymidine kinase, cytosine deaminase, B-cell CD20, ErbB2 or CD79b wherein when the genome-engineered iPSCs comprise two or more suicide genes, the suicide genes are integrated in different safe harbor locus comprising AAVS1, CCR5, ROSA26, collagen, HTRP, Hll, Hll, beta-2 microglobulin, GAPDH, TCR or RUNX1. Other exogenous polynucleotides encoding proteins may include those encoding PET reporters, homeostatic cytokines, and inhibitory checkpoint inhibitory proteins such as PD1, PD-L1, and CTLA4 as well as proteins that target the CD47/signal regulatory protein alpha (SIRPα) axis. In some other embodiments, the genome-engineered iPSCs generated using the method provided herein comprise in/del at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof.

V. Targeted Genome Editing at Selected Locus in iPSCs

According to embodiments of the application, one or more of the exogenous polynucleotides are integrated at one or more loci on the chromosome of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

Available endonucleases capable of introducing specific and targeted DSBs include, but not limited to, zinc-finger nucleases (ZEN), transcription activator-like effector nucleases (TALEN), RNA-guided CRISPR (Clustered Regular Interspaced Short Palindromic Repeats) systems. Additionally, DICE (dual integrase cassette exchange) system utilizing phiC31 and Bxbl integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Patent Application No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a targeted Spoll nuclease, a polypeptide comprising a Spol 1 polypeptide having nuclease activity fused to a DNA binding domain, e.g. a zinc finger DNA binding domain, a TAL effector DNA binding domain, etc. that has specificity for a DNA sequence of interest. See, for example, U.S. Application No. 61/555,857, the disclosure of which is incorporated herein by reference.

Additional examples of targeted nucleases suitable for the present application include, but not limited to Bxbl, phiC3 1, R4, PhiBT1, and Wp/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like. As an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. As another example, CRISPR/Cpf1 comprises two major components: (1) a CPf1 endonuclease and (2) a crRNA. When co-expressed, the two components form a ribobnucleoprotein (RNP) complex that is recruited to a target DNA sequence comprising PAM and a seeding region near PAM. The crRNA can be combined to form a chimeric guide RNA (gRNA) to guide Cpf1 to target selected sequences. These two components can then be delivered to mammalian cells via transfection or transduction.

MAD7 is an engineered Cas12a variant originating from the bacterium Eubacterium rectale that has a preference for 5′-TTTN-3′ and 5′-CTTN-3′ PAM sites and does not require a tracrRNA. See, for example, PCT Publication No. 2018/236548, the disclosure of which is incorporated herein by reference.

DICE mediated insertion uses a pair of recombinases, for example, phiC31 and Bxbl, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, U.S. Application Publication No. 2015/0140665, the disclosure of which is incorporated herein by reference.

One aspect of the present application provides a construct comprising one or more exogenous polynucleotides for targeted genome integration. In one embodiment, the construct further comprises a pair of homologous arm specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cpf1 expression cassette, and a gRNA comprising a guide sequence specific to a desired integration site to the cell to enable a Cpf1-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Sites for targeted integration include, but are not limited to, genomic safe harbors, which are intragenic or extragenic regions of the human genome that, theoretically, are able to accommodate predictable expression of newly integrated DNA without adverse effects on the host cell or organism. In certain embodiments, the genome safe harbor for the targeted integration is one or more loci of genes selected from the group consisting of AAVS1, CCR5, ROSA26, collagen, HTRP, Hll, GAPDH, TCR and RUNX1 genes.

In other embodiments, the site for targeted integration is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for target deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MEW complex comprising a MEW class I protein heterodimer and/or a MEW class II heterodimer, such that the diminished or reduced level is less than the level naturally detectable by other cells or by synthetic methods. MHC class I deficiency can be achieved by functional deletion of any region of the MHC class I locus (chromosome 6p21), or deletion or reducing the expression level of one or more MEW class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are integrated at one or more loci of genes selected from the group consisting of B2M, TAP 1, TAP 2, Tapasin, RFXANK, CIITA, RFX5 and RFXAP genes to thereby delete or reduce the expression of the gene(s) with the integration.

In certain embodiments, the exogenous polynucleotides are integrated at one or more loci on the chromosome of the cell, preferably the one or more loci are of genes selected from the group consisting of AAVS1, CCR5, ROSA26, collagen, HTRP, H11, GAPDH, RUNX1, B2M, TAPI, TAP2, Tapasin, NLRC5, CIITA, RFXANK, CIITA, RFX5, RFXAP, TCR a orb constant region, NKG2A, NKG2D, CD38, CIS, CBL-B, SOCS2, PD1, CTLA4, LAG3, TIM3, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. Preferably, the one or more exogenous polynucleotides are integrated at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, collagen, HTRP, Hll, GAPDH, TCR and RUNX1 genes. More preferably, the one or more of the exogenous polynucleotides are integrated at the loci of CIITA, AAVS1 and B2M genes.

In certain embodiments, (i) the first exogenous polynucleotide is integrated at a locus of AAVS1 gene; (ii) the second exogenous polypeptide is integrated at a locus of CIITA gene; and (iii) the third exogenous polypeptide is integrated at a locus of B2M gene; wherein integrations of the exogenous polynucleotides delete or reduce expression of CIITA and B2M genes.

In certain embodiments, (i) the first exogenous polynucleotide comprises the polynucleotide sequence having at least 90%, 91%, 92%, 93%, 94%, 95%, 96%, 97%, 98%, 99% or 100% sequence identity to SEQ ID NO: 62; (ii) the second exogenous polynucleotide comprises the polynucleotide sequence having at least 90%, 91%, 92%, 93%, 94%, 95%, 96%, 97%, 98%, 99% or 100% sequence identity to SEQ ID NO: 75; and (iii) the third exogenous polynucleotide comprises the polynucleotide sequence having at least 90%, 91%, 92%, 93%, 94%, 95%, 96%, 97%, 98%, 99% or 100% sequence identity to SEQ ID NO: 67.

In certain embodiments, (i) the first exogenous polynucleotide comprises the polynucleotide sequence of SEQ ID NO: 62; (ii) the second exogenous polynucleotide comprises the polynucleotide sequence of SEQ ID NO: 75; and (iii) the third exogenous polynucleotide comprises the polynucleotide sequence of SEQ ID NO: 67.

### Derivative Cells

In another aspect, the invention relates to a cell derived from differentiation of an iPSC, a derivative cell. As described above, the genomic edits introduced into the iPSC cell are retained in the derivative cell. In certain embodiments of the derivative cell obtained from iPSC differentiation, the derivative cell is a hematopoietic cell, including, but not limited to, HSCs (hematopoietic stem and progenitor cells), hematopoietic multipotent progenitor cells, T cell progenitors, NK cell progenitors, T cells, NKT cells, NK cells, B cells, antigen presenting cells (APC), monocytes and macrophages. In certain embodiments, the derivative cell is an immune effector cell, such as a NK cell or a T cell.

In certain embodiments, the application provides a natural killer (NK) cell or a T cell comprising: (i) a first exogenous polynucleotide encoding a chimeric antigen receptor (CAR); (ii) a second exogenous polynucleotide encoding a truncated epithelial growth factor (tEGFR) variant and an interleukin 15 (IL-15), wherein the tEGFR variant and IL-15 are operably linked by an autoprotease peptide sequence, such as autoprotease peptide sequence of porcine tesehovirus-1 2A (P2A); and (iii) a deletion or reduced expression of an MHC class I associated gene and an MHC class II associated gene, such as an MHC class-I associated gene selected from the group consisting of a B2M gene, TAP 1 gene, TAP 2 gene and Tapasin gene, and an MHC-II associated gene selected from the group consisting of a RFXANK gene, CIITA gene, RFX5 gene, RFXAP gene, and CIITA gene, preferably the B2M gene and CIITA gene.

In certain embodiments, the NK cell or T cell further comprises a third exogenous polynucleotide encoding at least one of a human leukocyte antigen E (HLA-E) and a human leukocyte antigen G (HLA-G).

Also provided is a NK cell or a T cell comprising: (i) a first exogenous polynucleotide encoding a chimeric antigen receptor (CAR) having the amino acid sequence of SEQ ID NO: 61; (ii) a second exogenous polynucleotide encoding a truncated epithelial growth factor (tEGFR) variant having the amino acid sequence of SEQ ID NO: 71, an autoprotease peptide having the amino acid sequence of SEQ ID NO: 73, and interleukin 15 (IL-15) having the amino acid sequence of SEQ ID NO: 72; and (iii) a third exogenous polynucleotide encoding a human leukocyte antigen E (HLA-E) having the amino acid sequence of SEQ ID NO: 66;

wherein the first, second and third exogenous polynucleotides are integrated at loci of AAVS1, CIITA and B2M genes, respectively, to thereby delete or reduce expression of CIITA and B2M.

In certain embodiments, the first exogenous polynucleotide comprises the polynucleotide sequence of SEQ ID NO: 62; the second exogenous polynucleotide comprises the polynucleotide sequence of SEQ ID NO: 75; and the third exogenous polynucleotide comprises the polynucleotide sequence of SEQ ID NO: 67.

Also provided is a CD34+ hematopoietic progenitor cell (HPC) derived from an induced pluripotent stem cell (iPSC) comprising: (i) a first exogenous polynucleotide encoding a chimeric antigen receptor (CAR); (ii) a second exogenous polynucleotide encoding an inactivated cell surface receptor that comprises a monoclonal antibody-specific epitope and an interleukin 15 (IL-15), wherein the inactivated cell surface receptor and IL-15 are operably linked by an autoprotease peptide sequence; and (iii) a deletion or reduced expression of one or more of B2M, TAP 1, TAP 2, Tapasin, RFXANK, CIITA, RFX5 and RFXAP genes.

In certain embodiments, the CD34+ HPC further comprises a third exogenous polynucleotide encoding a human leukocyte antigen E (HLA-E) and/or human leukocyte antigen G (HLA-G).

In certain embodiments, the CAR comprises (i) a signal peptide; (ii) an extracellular domain comprising a binding domain that specifically binds the CD19 antigen; (iii) a hinge region; (iv) a transmembrane domain; (v) an intracellular signaling domain; and (vi) a co-stimulatory domain, such as a co-stimulatory domain comprising a CD28 signaling domain.

Also provided is a method of manufacturing the derivative cell. The method comprises differentiating the iPSC under conditions for cell differentiation to thereby obtain the derivative cell.

An iPSC of the application can be differentiated by any method known in the art. Exemplary methods are described in U.S. Pat. Nos. 8,846,395, 8,945,922, 8,318,491, WO2010/099539, WO2012/109208, WO2017/070333, WO2017/179720, WO2016/010148, WO2018/048828 and WO2019/157597, each of which are herein incorporated by reference in its entirety. The differentiation protocol may use feeder cells or may be feeder-free. As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type.

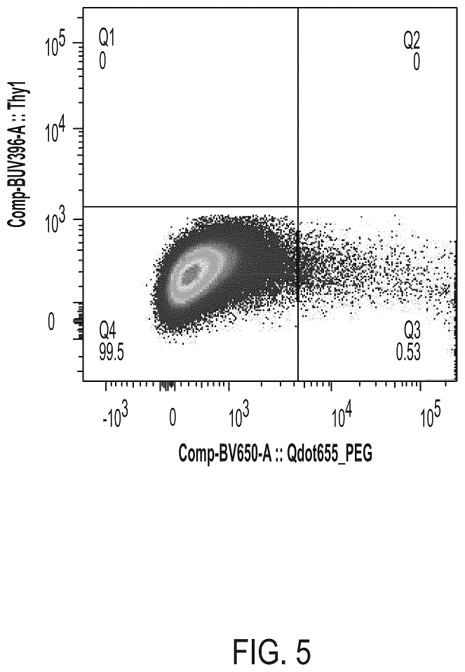

In another embodiment of the invention, the iPSC derivative cells of the invention are NK cells which are prepared by a method of differentiating an iPSC cell into an NK cell by subjecting the cells to a differentiation protocol including the addition of recombinant human IL-12p70 for the final 24 hours of culture. By including the IL-12 in the differentiation protocol, cells that are primed with IL-12 demonstrate more rapid cell killing compared to those that are differentiated in the absence of IL-12 (FIG. 5A). In addition, the cells differentiated using the IL-12 conditions demonstrate improved cancer cell growth inhibition (FIG. 5B).

### Polynucleotides, Vectors, and Host Cells

(1) Nucleic Acids Encoding a CAR

In another general aspect, the invention relates to an isolated nucleic acid encoding a chimeric antigen receptor (CAR) useful for an invention according to embodiments of the application. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARs of the application can be altered without changing the amino acid sequences of the proteins.

In certain embodiments, the isolated nucleic acid encodes a CAR targeting CD19. In a particular embodiment, the isolated nucleic acid encoding the CAR comprises a polynucleotide sequence at least 90%, such as at least 90%, 91%, 92%, 93%, 94%, 95%, 96%, 97%, 98% or 100%, identical to SEQ ID NO: 62, preferably the polynucleotide sequence of SEQ ID NO: 62.

In another general aspect, the application provides a vector comprising a polynucleotide sequence encoding a CAR useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the application.

In a particular aspect, the application provides vectors for targeted integration of a CAR useful for an invention according to embodiments of the application. In certain embodiments, the vector comprises an exogenous polynucleotide having, in the 5′ to 3′ order, (a) a promoter; (b) a polynucleotide sequence encoding a CAR according to an embodiment of the application; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to, BGH, hGH, and PGK.

In certain embodiments, the polynucleotide sequence encoding a CAR comprises the polynucleotide sequence at least 90%, such as at least 90%, 91%, 92%, 93%, 94%, 95%, 96%, 97%, 98% or 100%, identical to SEQ ID NO: 62.

In some embodiment, the vector further comprises a left homology arm and a right homology arm flanking the exogenous polynucleotide. As used herein, “left homology arm” and “right homology arm” refers to a pair of nucleic acid sequences that flank an exogenous polynucleotide and facilitate the integration of the exogenous polynucleotide into a specified chromosomal locus. Sequences of the left and right arm homology arms can be designed based on the integration site of interest. In some embodiment, the left or right arm homology arm is homologous to the left or right side sequence of the integration site.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 90. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 91.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 92, preferably the polynucleotide sequence of SEQ ID NO: 92.

(2) Nucleic Acids Encoding an Inactivated Cell Surface Receptor

In another general aspect, the invention relates to an isolated nucleic acid encoding an inactivated cell surface receptor useful for an invention according to embodiments of the application. It will be appreciated by those skilled in the art that the coding sequence of an inactivated cell surface receptor can be changed (e.g., replaced, deleted, inserted, etc.) without changing the amino acid sequence of the protein. Accordingly, it will be understood by those skilled in the art that nucleic acid sequences encoding an inactivated cell surface receptor of the application can be altered without changing the amino acid sequences of the proteins.

In certain embodiments, an isolated nucleic acid encodes any inactivated cell surface receptor described herein, such as that comprises a monoclonal antibody-specific epitope, and a cytokine, such as an IL-15 or IL-2, wherein the monoclonal antibody-specific epitope and the cytokine are operably linked by an autoprotease peptide sequence.

In some embodiments, the isolated nucleic acid encodes an inactivated cell surface receptor comprising an epitope specifically recognized by an antibody, such as ibritumomab, tiuxetan, muromonab-CD3, tositumomab, abciximab, basiliximab, brentuximab vedotin, cetuximab, infliximab, rituximab, alemtuzumab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, or ustekinumab.

In certain embodiments, the isolated nucleic acid encodes an inactivated cell surface receptor having a truncated epithelial growth factor (tEGFR) variant. Preferably, the inactivated cell surface receptor comprises an epitope specifically recognized by cetuximab, matuzumab, necitumumab or panitumumab, preferably cetuximab.

In certain embodiments, the isolated nucleic acid encodes an inactivated cell surface receptor having one or more epitopes of CD79b, such as an epitope specifically recognized by polatuzumab vedotin.

In certain embodiments, the isolated nucleic acid encodes an inactivated cell surface receptor having one or more epitopes of CD20, such as an epitope specifically recognized by rituximab.

In certain embodiments, the isolated nucleic acid encodes an inactivated cell surface receptor having one or more epitopes of Her 2 receptor, such as an epitope specifically recognized by trastuzumab

In certain embodiments, the autoprotease peptide sequence is porcine tesehovirus-1 2A (P2A).

In certain embodiments, the truncated epithelial growth factor (tEGFR) variant consists of an amino acid sequence having at least 90%, 91%, 92%, 93%, 94%, 95%, 96%, 97%, 98%, 99% or 100% sequence identity to the amino acid sequence of SEQ ID NO: 71.

In certain embodiments, the monoclonal antibody-specific epitope specifically recognized by polatuzumab vedotin consists of an amino acid sequence at least 90%, such as at least 90%, 91%, 92%, 93%, 94%, 95%, 96%, 97%, 98%, 99% or 100%, identical to SEQ ID NO: 78.

In certain embodiments, the monoclonal antibody-specific epitope specifically recognized by rituximab consists of an amino acid sequence at least 90%, such as at least 90%, 91%, 92%, 93%, 94%, 95%, 96%, 97%, 98%, 99% or 100%, identical to SEQ ID NO: 80.

In certain embodiments, the monoclonal antibody-specific epitope specifically recognized by trastuzumab consists of an amino acid sequence at least 90%, such as at least 90%, 91%, 92%, 93%, 94%, 95%, 96%, 97%, 98%, 99% or 100%, identical to SEQ ID NO: 82.

In certain embodiments, the IL-15 comprises an amino acid sequence having at least 90%, 91%, 92%, 93%, 94%, 95%, 96%, 97%, 98%, 99% or 100% sequence identity to the amino acid sequence of SEQ ID NO: 72.

In certain embodiments, the autoprotease peptide has an amino acid sequence having at least 90%, 91%, 92%, 93%, 94%, 95%, 96%, 97%, 98%, 99% or 100% sequence identity to the amino acid sequence of SEQ ID NO: 73.

In certain embodiments, the polynucleotide sequence encodes a polypeptide comprising an amino acid sequence having at least 90%, 91%, 92%, 93%, 94%, 95%, 96%, 97%, 98%, 99% or 100% sequence identity to the amino acid sequence of SEQ ID NO: 74.

In a particular embodiment, the isolated nucleic acid encoding the inactivated cell surface receptor comprises a polynucleotide sequence at least 90%, such as at least 90%, 91%, 92%, 93%, 94%, 95%, 96%, 97%, 98% or 100%, identical to SEQ ID NO: 75, preferably the polynucleotide sequence of SEQ ID NO: 75.

In certain embodiments, the polynucleotide sequence encodes a polypeptide comprising an amino acid sequence having at least 90%, 91%, 92%, 93%, 94%, 95%, 96%, 97%, 98%, 99% or 100% sequence identity to the amino acid sequence of SEQ ID NO: 79.

In another general aspect, the application provides a vector comprising a polynucleotide sequence encoding an inactivated cell surface receptor useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a inactivated cell surface receptor in the cell. Conventional cloning techniques or artificial gene synthesis can be used to generate a recombinant expression vector according to embodiments of the application.

In a particular aspect, the application provides a vector for targeted integration of an inactivated cell surface receptor useful for an invention according to embodiments of the application. In certain embodiments, the vector comprises an exogenous polynucleotide having, in the 5′ to 3′ order, (a) a promoter; (b) a polynucleotide sequence encoding an inactivated cell surface receptor, such as an inactivated cell surface receptor comprising a truncated epithelial growth factor (tEGFR) variant and an interleukin 15 (IL-15), wherein the tEGFR variant and IL-15 are operably linked by an autoprotease peptide sequence, such as porcine tesehovirus-1 2A (P2A), and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, the polynucleotide sequence encoding an inactivated cell surface receptor comprises the polynucleotide sequence at least 90%, such as at least 90%, 91%, 92%, 93%, 94%, 95%, 96%, 97%, 98% or 100%, identical to SEQ ID NO: In some embodiment, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 84. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 85

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 86, preferably the polynucleotide sequence of SEQ ID NO: 86.

(3) Nucleic Acids Encoding an HLA Construct

In another general aspect, the invention relates to an isolated nucleic acid encoding an HLA construct useful for an invention according to embodiments of the application. It will be appreciated by those skilled in the art that the coding sequence of an HLA construct can be changed (e.g., replaced, deleted, inserted, etc.) without changing the amino acid sequence of the protein. Accordingly, it will be understood by those skilled in the art that nucleic acid sequences encoding an HLA construct of the application can be altered without changing the amino acid sequences of the proteins.

In certain embodiments, the isolated nucleic acid encodes an HLA construct comprising a signal peptide, such as an HLA-G signal peptide, operably linked to an HLA coding sequence, such as a coding sequence of a mature B2M, and/or a mature HLA-E. In some embodiments, the HLA coding sequence encodes the HLA-G and B2M, which are operably linked by a 4× GGGGS linker, and/or the B2M and HLA-E, which are operably linked by a 3× GGGGS linker. In a particular embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In another general aspect, the application provides a vector comprising a polynucleotide sequence encoding a HLA construct useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a HLA construct in the cell. Conventional cloning techniques or artificial gene synthesis can be used to generate a recombinant expression vector according to embodiments of the application.

In a particular aspect, the application provides vectors for targeted integration of a HLA construct useful for an invention according to embodiments of the application. In certain embodiments, the vector comprises an exogenous polynucleotide having, in the 5′ to 3′ order, (a) a promoter; (b) a polynucleotide sequence encoding an HLA construct; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EF1a, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, a polynucleotide sequence encoding a HLA construct comprises a signal peptide, such as a HLA-G signal peptide, a mature B2M, and a mature HLA-E, wherein the HLA-G and B2M are operably linked by a 4× GGGGS linker (SEQ ID NO: 31) and the B2M transgene and HLA-E are operably linked by a 3× GGGGS linker (SEQ ID NO: 25). In particular embodiments, the HLA construct comprises the polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the HLA construct comprises the polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In some embodiment, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 87. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 88.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 89, preferably the polynucleotide sequence of SEQ ID NO: 89.

(4) Host Cells

In another general aspect, the application provides a host cell comprising a vector of the application and/or an isolated nucleic acid encoding a construct of the application. Any host cell known to those skilled in the art in view of the present disclosure can be used for recombinant expression of exogenous polynucleotides of the application. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Examples of host cells include, for example, recombinant cells containing a vector or isolated nucleic acid of the application useful for the production of a vector or construct of interest; or an engineered iPSC or derivative cell thereof containing one or more isolated nucleic acids of the application, preferably integrated at one or more chromosomal loci. A host cell of an isolated nucleic acid of the application can also be an immune effector cell, such as a T cell or NK cell, comprising the one or more isolated nucleic acids of the application. The immune effector cell can be obtained by differentiation of an engineered iPSC of the application. Any suitable method in the art can be used for the differentiation in view of the present disclosure. The immune effector cell can also be obtained transfecting an immune effector cell with one or more isolated nucleic acids of the application.

### Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (WED).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g., 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the application.

### Methods of Use

Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumour, a “clinically detectable” tumour is one that is detectable on the basis of tumour mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient.

Cancer conditions may be characterized by the abnormal proliferation of malignant cancer cells and may include leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreatic cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumour may be immunogenic). For example, the cancer cells may be capable of eliciting a systemic immune response in the individual against one or more antigens expressed by the cancer cells. The tumour antigens that elicit the immune response may be specific to cancer cells or may be shared by one or more normal cells in the individual.

The cancer cells of an individual suitable for treatment as described herein may express the antigen and/or may be of correct HLA type to bind the antigen receptor expressed by the T cells.

An individual suitable for treatment as described above may be a mammal. In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

In some embodiments, the individual may have minimal residual disease (MRD) after an initial cancer treatment. In some embodiments, the individual may have no minimal residual disease after one or more cancer treatments or repeated dosing.

An individual with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in an individual may include identification of a particular cell type (e.g. a cancer cell) in a sample of a body fluid or tissue obtained from the individual.

An anti-tumor effect is a biological effect which can be manifested by a reduction in the rate of tumor growth, decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies, also T cells which may be obtained according to the methods of the present invention, as described herein in prevention of the occurrence of tumors in the first place.

In another general aspect, the application provides a method of treating a disease or a condition in a subject in need thereof. The methods comprise administering to the subject in need thereof a therapeutically effective amount of cells of the application and/or a composition of the application. In certain embodiments, the disease or condition is cancer. The cancer can, for example, be a solid or a liquid cancer. The cancer, can, for example, be selected from the group consisting of a lung cancer, a gastric cancer, a colon cancer, a liver cancer, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. In a preferred embodiment, the cancer is a non-Hodgkin's lymphoma (NHL).

Treatment may be any treatment and/or therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment may also be prophylactic (i.e. prophylaxis). For example, an individual susceptible to or at risk of the occurrence or re-occurrence of cancer may be treated as described herein. Such treatment may prevent or delay the occurrence or re-occurrence of cancer in the individual.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of T cells, and a decrease in levels of tumor-specific antigens. Administration of T cells modified as described herein may improve the capacity of the individual to resist cancer growth, in particular growth of a cancer already present the subject and/or decrease the propensity for cancer growth in the individual.

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered transarterially, subcutaneously, intradermaly, intratumorally, intranodally, intramedullary, intramuscularly, inrapleurally, by intravenous (i.v.) injection, or intraperitoneally. In certain embodiments, the cells of the application can be administered with or without lymphodepletion of the subject.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

Abbreviations

| | |
|---|---|
| PEG | Polyethylene glycol |
| CAR | Chimeric Antigen Receptor |
| Qdot | Quantum Dot |
| Fc | Fragment Crystallizable |
| Ig | Immunoglobulin |
| uM | Micromolar |
| nM | Nanomolar |
| UTD | untransduced |
| VHH | Single variable domain on a |
| | heavy chain |
| KO | Knockout |
| scFv | Single chain variable fragment |

### EXAMPLES

### Example 1. Nur77 Reporter Jurkat Transduction Using Anti-PEG CARs and Qdot Staining

The objective of this experiment was to express anti-PEG CARs in Jurkats and characterize binding to soluble PEG. Jurkat cells were transduced with lentivirus encoding anti-PEG CARs. These anti-PEG CAR constructs also included a murine Thy1.1 marker that served as a proxy for the assessment of the level of CAR expression in a transduced cell.

### Reagents

ViaStain™ AOPI Staining Solution in PBS (Cat #: CS2-0106-5 mL)
Nexcelom Cellometer Cell Counter and slides
Sterile polystyrene 5 mL FACS tubes
24-well TC treated plate; 6-well TC treated plate; 24 well GREX plate
BioLegend Cell Staining Buffer (CSB) (Cat #: 420201)
BioLegend Fixation Buffer (Cat #: 420801)
Steriflip Vacuum Filtration system with Millipore Express PLUS membrane (0.22 um) 50 mL (Cat #: SCGP00525)
R10 Cell line media
   RPMI 1640 (1×)+L-Glutamine (Cat #: 11875-093)
   Cytiva Fetal Bovine Serum Defined (Cat #: SH30070.03)

### Nur77 Jurkat Cell Transduction

Cells were transferred to either a 50 mL conical. Cells were pelleted by centrifugation at 1600 RPM for 4 mins for 50 mL tubes and 1200 RPM for 5 mins for 15 mL tubes. The supernatant was aspirated. Cells were counted and resuspend at 1×10⁶ cells/well in R10 media and plated at a density of 2.5×10⁵ cells per well in a 24 well plate.

Cells were transduced with one of the anti-PEG chimeric receptors shown in Table 12 using lentivirus. Briefly, lentivirus was thawed on ice, and added to each well of the 24 well plate, and the plate was gently swirled to mix. The plate was centrifuged at 32 degrees C. at 1300 g for 90 minutes. 750 uL of R10 media was added to each well, and the plate was placed in the incubator overnight at 37 degrees C./5% CO2.

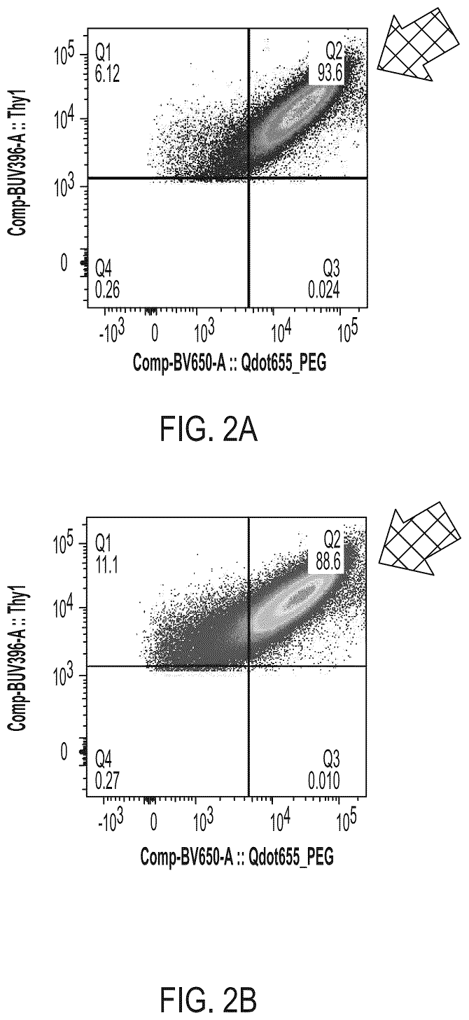

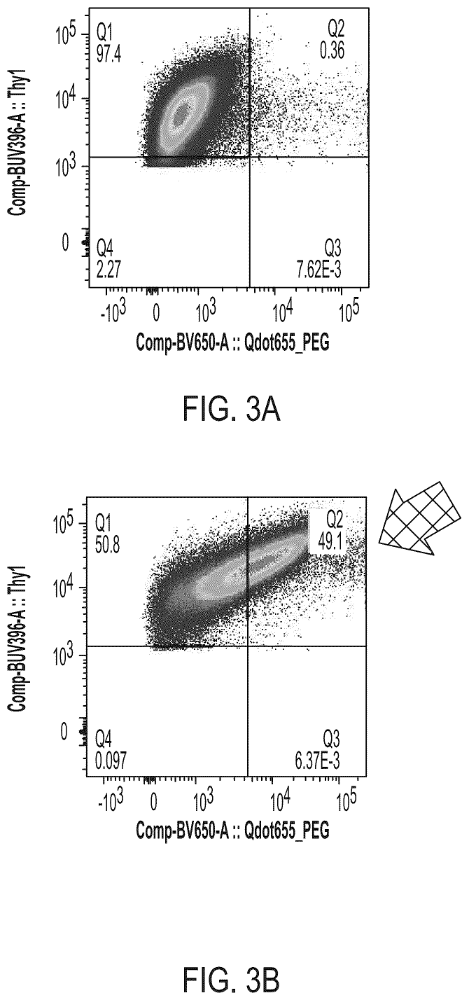

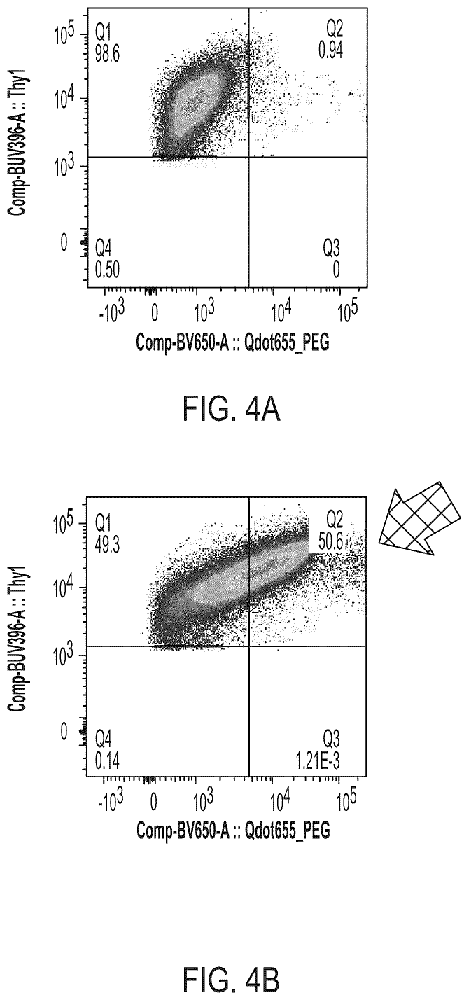

A minimum of three days later, cells were stained using fluorescently labeled antibodies specific for Thy1.1, Qdot655-PEG2K (ThermoFisher Cat #Q21521MP) and a viability dye for 20-30 mins at 4 degrees C. Using a flow cytometer, levels of Thy1.1 staining, Qdot655 staining, viability, and cell size/complexity were measured. As shown in FIGS. 2-5, data was analyzed using FlowJo software.

### Example 2. Activation of Nur77 Jurkat Cells Transduced with Anti-PEG CARs

The objective of this experiment was to determine whether PEG-Qdots could bind to and activate Nur77 Jurkat cells transduced with anti-PEG CARs. Jurkat cells that express green fluorescent protein under the control of the Nur77 promoter (e.g., where Nur77 expression leads to expression of GFP) were transduced with lentivirus encoding anti-PEG CARs as described in Example 1. These anti-PEG CAR constructs also included a murine Thy1.1 marker that allowed for the assessment of the level of CAR expression in a transduced cell.

### Reagents

R10 Cell line media
   RPMI 1640 (1×)+L-Glutamine (Cat #: 11875-093)
   Cytiva Fetal Bovine Serum Defined (Cat #: SH30070.03)
Cells
   Nur77-GFP Jurkats transduced with one of 4 different PEG constructs (Table 12; SEQ ID NOs: 178-181) or untransduced
Qdot655PEG2K (ThermoFisher; Cat #Q21521MP)
Flow cytometry
   dPBS+10% FBS, polypropylene tubes
   Stain with Thy1.1 (1:150 dilution) PE

### Cell Preparation & Activation

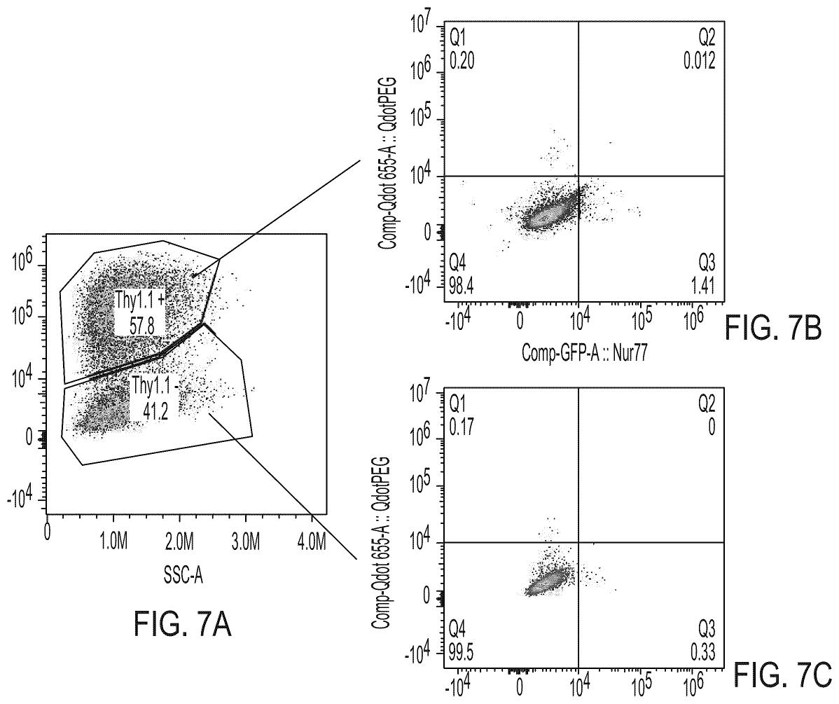

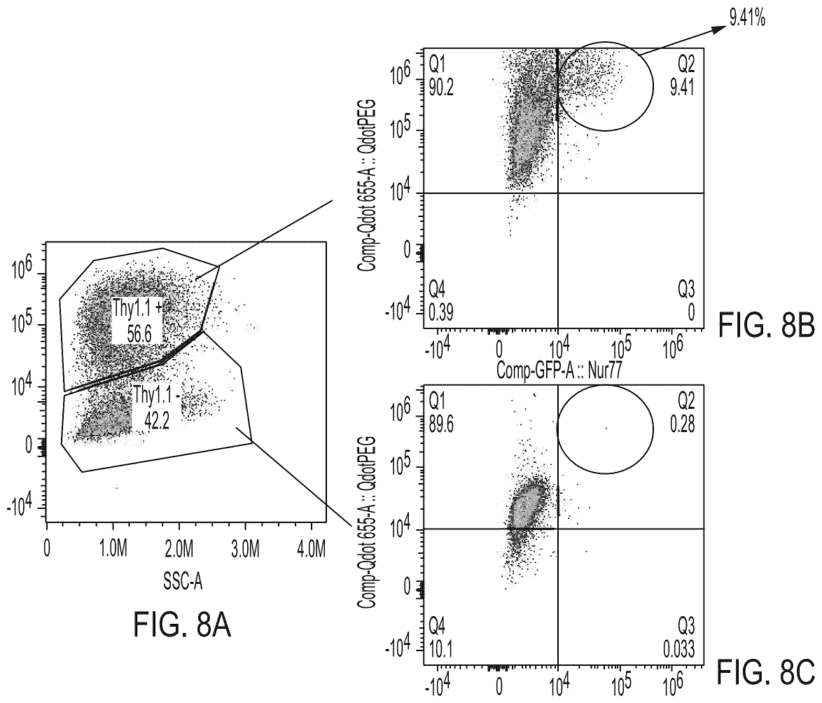

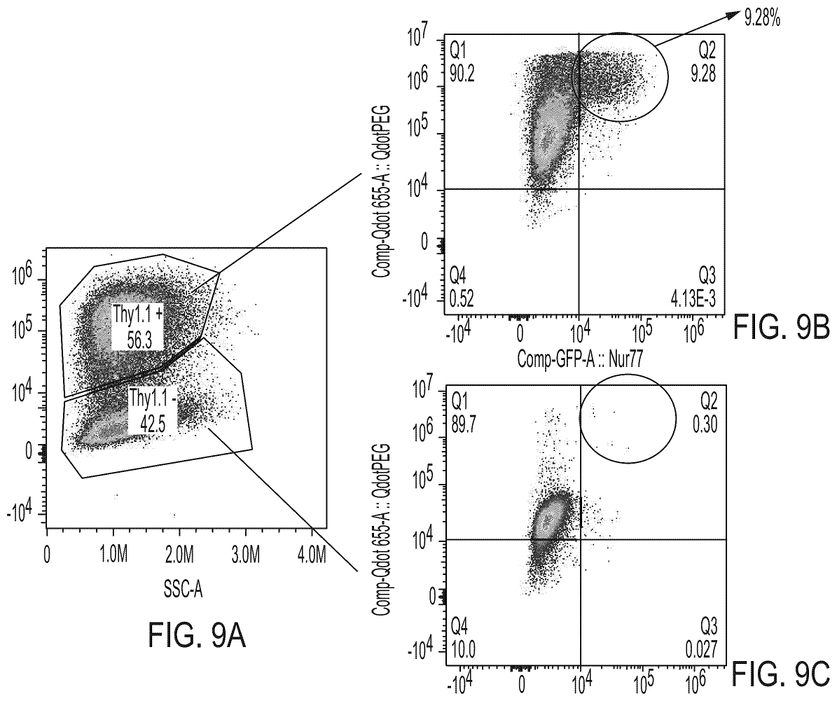

Transduced cells were suspended in R10 media and plated in a 96 well U-bottom plate at a density of about 4×10⁵ per well. Cells were subsequently incubated with Qdots. Briefly, the 96 well plate was spun down at 2000 g for 2 min. After aspirating the supernatant, transduced Jurkat cells from each of the different anti-PEG CAR groups were then co-cultured with Qdot655-PEG2K (ThermoFisher Cat #Q21521MP) that was diluted to 0 nM, 5 nM, or 10 nM concentration using sterile cell culturing media. Transduced Jurkat cells co-cultured with cell culture media alone served as negative controls. Transduced Jurkat cells co-cultured in Immunocult activation reagent diluted in cell culturing media served as positive controls. All cells were then incubated at 37 degrees C./5% CO2 for either 3.5 hrs or 22.5 hrs. After incubation, cells were stained using fluorescently labeled antibodies specific for Thy1.1 and a viability dye. Using a flow cytometer, levels of GFP expression, Thy1.1 staining, viability and cell size/complexity were measured. As shown in FIGS. 6-10, data was analyzed using FlowJo software.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

It is claimed:

1. An induced pluripotent stem cell (iPSC) or a derivative cell thereof comprising:
one or more first exogenous polynucleotides encoding:
an extracellular polyethylene glycol (PEG) recognition element operably linked to an intracellular signaling domain, and
a chimeric antigen receptor (CAR) or T-cell receptor (TCR) targeting a cancer antigen.

2. The iPSC or the derivative cell according to claim 1, further comprising at least one of:
(i) one or more second exogenous polynucleotides encoding an inactivated cell surface receptor that comprises a monoclonal antibody-specific epitope and an interleukin (IL-15), wherein the inactivated cell surface receptor and the IL-15 are operably linked by an autoprotease peptide; and
(ii) a deletion or reduced expression of one or more of B2M, TAP 1, TAP 2, Tapasin, RFXANK, CIITA, RFX5 and RFXAP genes.

3. The iPSC or the derivative cell according to claim 1, wherein the cancer antigen comprises CD19.

4. The iPSC or the derivative cell according to claim 3, wherein the one or more first exogenous polynucleotides encode an additional CAR or TCR targeting CD22 or CD79b.

5. The iPSC or the derivative cell according to claim 3, wherein the CAR comprises a bispecific CAR targeting the CD19 antigen and an additional antigen selected from the group consisting of CD22 and CD79b.

6. The iPSC or the derivative cell according to claim 1, wherein the CAR comprises a bispecific CAR targeting a CD133 antigen and an EGFR antigen.

7. The iPSC or the derivative cell according to claim 1, wherein the CAR comprises an antigen binding domain, and the antigen binding domain comprises an scFv or a VHH domain.

8. The iPSC or the derivative cell according to claim 1, wherein the extracellular PEG recognition element comprises an anti-PEG scFv or an anti-PEG VHH domain.

9. The iPSC or the derivative cell according to claim 8, wherein the anti-PEG scFV or the anti-PEG VHH domain is operably linked to (i) the intracellular signaling domain, and (ii) one or more of a signal peptide, a hinge, a spacer, a transmembrane domain, and a costimulatory domain, thereby forming a functional anti-PEG CAR.

10. The iPSC or the derivative cell according to claim 8, wherein the intracellular signaling domain comprises a cytoplasmic domain of a cytokine receptor, and wherein the anti-PEG scFV or the anti-PEG VHH domain is operably linked to a transmembrane domain and the cytoplasmic domain, thereby forming a chimeric cytokine receptor (CCR).

11. The iPSC or the derivative cell according to claim 10, wherein (i) the transmembrane domain comprises an IL-7Ra (CD127) transmembrane domain, or (ii) the cytoplasmic domain comprises an IL-7Ra (CD127) cytoplasmic domain.

12. (canceled)

13. The iPSC or the derivative cell according to claim 1, further comprising one or more third exogenous polynucleotides encoding a human leukocyte antigen E (HLA-E) and/or human leukocyte antigen G (HLA-G).

14. The iPSC or the derivative cell according to claim 9, wherein the PEG recognition element comprises one or more of:
(i) the signal peptide comprising an amino acid sequence having at least 90%, 91%, 92%, 93%, 94%, 95%, 96%, 97%, 98%, 99% or 100% sequence identity to SEQ ID NO: 103 or 145;
(ii) the anti-PEG scFV or the anti-PEG VHH comprising an amino acid sequence having at least 90%, 91%, 92%, 93%, 94%, 95%, 96%, 97%, 98%, 99% or 100% sequence identity to SEQ ID NO: 147, 149, 151, 153, 155, or 157;
(iii) the spacer comprising an amino acid sequence having at least 90%, 91%, 92%, 93%, 94%, 95%, 96%, 97%, 98%, 99% or 100% sequence identity to SEQ ID NO: 159 or 161;
(iv) the transmembrane domain comprising an amino acid sequence having at least 90%, 91%, 92%, 93%, 94%, 95%, 96%, 97%, 98%, 99% or 100% sequence identity to SEQ ID NO: 24 or 164;
(v) the co-stimulatory domain comprising an amino acid sequence having at least 90%, 91%, 92%, 93%, 94%, 95%, 96%, 97%, 98%, 99% or 100% sequence identity to SEQ ID NO: 8;
(vi) the activation domain comprising an amino acid sequence having at least 90%, 91%, 92%, 93%, 94%, 95%, 96%, 97%, 98%, 99% or 100% sequence identity to SEQ ID NO: 6;
(vii) the cytoplasmic domain comprising an amino acid sequence having at least 90%, 91%, 92%, 93%, 94%, 95%, 96%, 97%, 98%, 99% or 100% sequence identity to SEQ ID NO: 168;
(viii) a 2A peptide sequence comprising an amino acid sequence having at least 90%, 91%, 92%, 93%, 94%, 95%, 96%, 97%, 98%, 99% or 100% sequence identity to SEQ ID NO: 170, 172, or 173; and
(ix) a staining handle/reporter comprising an amino acid sequence having at least 90%, 91%, 92%, 93%, 94%, 95%, 96%, 97%, 98%, 99% or 100% sequence identity to SEQ ID NO: 174 or 176.

15. The iPSC or the derivative cell according to claim 9, wherein the PEG recognition element comprises one or more of:
(i) the signal peptide encoded by a nucleic acid sequence having at least 90%, 91%, 92%, 93%, 94%, 95%, 96%, 97%, 98%, 99% or 100% sequence identity to SEQ ID NO: 144 or 146;
(ii) the anti-PEG scFV or the anti-PEG VHH encoded by a nucleic acid sequence having at least 90%, 91%, 92%, 93%, 94%, 95%, 96%, 97%, 98%, 99% or 100% sequence identity to SEQ ID NO: 148, 150, 152, 154, 156, or 158;
(iii) the spacer encoded by a nucleic acid sequence having at least 90%, 91%, 92%, 93%, 94%, 95%, 96%, 97%, 98%, 99% or 100% sequence identity to SEQ ID NO: 160 or 162;
(iv) the transmembrane domain encoded by a nucleic acid sequence having at least 90%, 91%, 92%, 93%, 94%, 95%, 96%, 97%, 98%, 99% or 100% sequence identity to SEQ ID NO: 163 or 165;
(v) the co-stimulatory domain encoded by a nucleic acid sequence having at least 90%, 91%, 92%, 93%, 94%, 95%, 96%, 97%, 98%, 99% or 100% sequence identity to SEQ ID NO: 166;
(vi) the activation domain encoded by a nucleic acid sequence having at least 90%, 91%, 92%, 93%, 94%, 95%, 96%, 97%, 98%, 99% or 100% sequence identity to SEQ ID NO: 167;
(vii) the cytoplasmic domain encoded by a nucleic acid sequence having at least 90%, 91%, 92%, 93%, 94%, 95%, 96%, 97%, 98%, 99% or 100% sequence identity to SEQ ID NO: 169;
(viii) a 2A peptide sequence encoded by a nucleic acid sequence having at least 90%, 91%, 92%, 93%, 94%, 95%, 96%, 97%, 98%, 99% or 100% sequence identity to SEQ ID NO: 171; and
(ix) a staining handle/reporter encoded by a nucleic acid sequence having at least 90%, 91%, 92%, 93%, 94%, 95%, 96%, 97%, 98%, 99% or 100% sequence identity to SEQ ID NO: 175 or 177.

16. The iPSC or the derivative cell according to claim 13, wherein one or more of the first, the second, and/or the third exogenous polynucleotides are integrated at one or more loci on the chromosome of the cell selected from the group consisting of AAVS1, CCR5, ROSA26, collagen, HTRP, HI 1, GAPDH, RUNX1, B2M, TAPI, TAP2, Tapasin, NLRC5, RFXANK, CIITA, RFX5, RFXAP, TCR a orb constant region, NKG2A, NKG2D, CD38, CIS, CBL-B, SOCS2, PD1, CTLA4, LAG3, TIM3, and TIGIT genes, provided at least one of the exogenous polynucleotides is integrated at a locus of a gene selected from the group consisting of B2M, TAP 1, TAP 2, Tapasin, RFXANK, CIITA, RFX5 and RFXAP genes to thereby result in a deletion or reduced expression of the gene.

17. The iPSC or the derivative cell according to claim 13, wherein one or more of the first, the second, and/or the third exogenous polynucleotides are integrated at the loci of the CIITA, AAVS1 and B2M genes.

18. The iPSC or the derivative cell according to claim 1, comprising a deletion or reduced expression of one or more of B2M or CIITA genes.

19. (canceled)

20. (canceled)

21. The iPSC or the derivative cell according to claim 2, wherein the CAR comprises:
(i) a signal peptide;
(ii) an extracellular domain comprising a binding domain that specifically binds the CD19 antigen;
(iii) a hinge region;
(iv) a transmembrane domain,
(v) an intracellular signaling domain; and
(vi) a co-stimulatory domain.

22. The iPSC or the derivative cell according to claim 21, wherein the extracellular domain comprises an scFv derived from an antibody that specifically binds the CD19 antigen.

23. The iPSC or the derivative cell according to claim 21 or 22, wherein the extracellular domain comprising an amino acid sequence having at least 90%, 91%, 92%, 93%, 94%, 95%, 96%, 97%, 98%, 99% or 100% sequence identity to SEQ ID NO: 7.

24.-28. (canceled)

29. The iPSC or the derivative cell according to claim 21, wherein the CAR comprises:
(i) the signal peptide comprising an amino acid sequence having at least 90%, 91%, 92%, 93%, 94%, 95%, 96%, 97%, 98%, 99% or 100% sequence identity to SEQ ID NO: 1;
(ii) the extracellular domain comprising an amino acid sequence having at least 90%, 91%, 92%, 93%, 94%, 95%, 96%, 97%, 98%, 99% or 100% sequence identity to SEQ ID NO: 7;
(iii) the hinge region comprising an amino acid sequence having at least 90%, 91%, 92%, 93%, 94%, 95%, 96%, 97%, 98%, 99% or 100% sequence identity to SEQ ID NO: 22;
(iv) the transmembrane domain comprising an amino acid sequence having at least 90%, 91%, 92%, 93%, 94%, 95%, 96%, 97%, 98%, 99% or 100% sequence identity to SEQ ID NO: 24;
(v) the intracellular signaling domain comprising an amino acid sequence having at least 90%, 91%, 92%, 93%, 94%, 95%, 96%, 97%, 98%, 99% or 100% sequence identity to SEQ ID NO: 6; and
(vi) the co-stimulatory domain comprising an amino acid sequence having at least 90%, 91%, 92%, 93%, 94%, 95%, 96%, 97%, 98%, 99% or 100% sequence identity to SEQ ID NO: 20.

30. The iPSC or the derivative cell according to claim 21, wherein the CAR comprises:
(i) the signal peptide comprising the amino acid sequence of SEQ ID NO: 1;
(ii) the extracellular domain comprising the amino acid sequence of SEQ ID NO: 7;
(iii) the hinge region comprising the amino acid sequence of SEQ ID NO: 22;
(iv) the transmembrane domain comprising the amino acid sequence of SEQ ID NO: 24;
(v) the intracellular signaling domain comprising the amino acid sequence of SEQ ID NO: 6; and
(vi) the co-stimulatory domain comprising the amino acid sequence of SEQ ID NO: 20.

31.-37. (canceled)

38. The iPSC or the derivative cell according to claim 13, wherein:
(i) the one or more first exogenous polynucleotides comprise the polynucleotide sequence having at least 90%, 91%, 92%, 93%, 94%, 95%, 96%, 97%, 98%, 99% or 100% sequence identity to one or more polynucleotide sequences selected from the group consisting of SEQ ID NOs: 62, 99-101, 112-119, and 132-143;
(ii) the one or more second exogenous polynucleotides comprise the polynucleotide sequence having at least 90%, 91%, 92%, 93%, 94%, 95%, 96%, 97%, 98%, 99% or 100% sequence identity to SEQ ID NO: 75; and
(iii) the one or more third exogenous polynucleotides comprise the polynucleotide sequence having at least 90%, 91%, 92%, 93%, 94%, 95%, 96%, 97%, 98%, 99% or 100% sequence identity to SEQ ID NO: 67.

39. The iPSC or the derivative cell according to claim 38, wherein:
(i) the one or more first exogenous polynucleotides is integrated at a locus of AAVS1 gene;
(ii) the one or more second exogenous polynucleotides is integrated at a locus of CIITA gene; and
(iii) the one or more third exogenous polynucleotides is integrated at a locus of B2M gene;
wherein integration of the exogenous polynucleotides deletes or reduces expression of CIITA and B2M, preferably, the one or more first exogenous polynucleotides comprises one or more of the polynucleotide sequences of SEQ ID NOs:62, 99-101, 112-119, and 132-143, the second exogenous polynucleotide comprises the polynucleotide sequence of SEQ ID NO: 75, and the third exogenous polynucleotide comprises the polynucleotide sequence of SEQ ID NO: 67.

40. The iPSC or the derivative cell according to claim 5 comprising the bispecific CAR, wherein the bispecific CAR comprises one or more amino acid sequences having at least 90%, 91%, 92%, 93%, 94%, 95%, 96%, 97%, 98%, 99% or 100% sequence identity to a sequence selected from the group consisting of SEQ ID NOs: 61, 96-98, 104-111, and 120-131.

41. The iPSC or the derivative cell according to claim 5 comprising the bispecific CAR, wherein the bispecific CAR comprises one or more polynucleotide sequences having at least 90%, 91%, 92%, 93%, 94%, 95%, 96%, 97%, 98%, 99% or 100% sequence identity to a sequence selected from the group consisting of SEQ ID NOs: 62, 99-101, 112-119, and 132-143.

42. The derivative cell of claim 1, wherein the derivative cell is a natural killer (NK) cell or a T cell.

43. (canceled)

44. An induced pluripotent stem cell (iPSC), a natural killer (NK) cell or a T cell comprising:
(i) one or more first exogenous polynucleotides encoding:
an extracellular polyethylene glycol (PEG) recognition element operably linked to an intracellular signaling domain having one or more amino acid sequences selected from the group consisting of SEQ ID NOs: 178-186, and
a chimeric antigen receptor (CAR) or T-cell receptor (TCR);
(ii) a second exogenous polynucleotide encoding a truncated epithelial growth factor (tEGFR) variant having the amino acid sequence of SEQ ID NO: 71, an autoprotease peptide having the amino acid sequence of SEQ ID NO: 73, and interleukin 15 (IL-15) having the amino acid sequence of SEQ ID NO: 72; and
(iii) optionally, a third exogenous polynucleotide encoding a human leukocyte antigen E (HLA-E) having the amino acid sequence of SEQ ID NO: 66;
wherein the first, second and third exogenous polynucleotides are integrated at loci of AAVS1, CIITA and B2M genes, to thereby delete or reduce expression of CIITA and B2M.

45. The iPSC, NK cell or T cell according to claim 44, wherein:
(i) the one or more first exogenous polynucleotide comprises one or more polynucleotide sequences selected from the group consisting of SEQ ID NOs: 148, 150, 152, 154, 156, and 158;
(ii) the second exogenous polynucleotide comprises the polynucleotide sequence of SEQ ID NO: 75; and
(iii) the third exogenous polynucleotide comprises the polynucleotide sequence of SEQ ID NO: 67, and
the first, second and third exogenous polynucleotides are integrated at loci of AAVS1, CIITA and B2M genes, respectively.

46.-53. (canceled)

54. A method of expanding and/or activating the iPSC or the derivative cell according to claim 1, comprising contacting the iPSC or the derivative cell with a predetermined amount of PEG.

55. A method of manufacturing the derivative cell according to claim 1, comprising differentiating the iPSC cell under conditions for cell differentiation to thereby obtain the derivative cell.

56. (canceled)

57. (canceled)

58. A method of differentiating an induced pluripotent stem cell (iPSC) of claim 1 into an NK cell, comprising subjecting the iPSCs to a differentiation protocol including culturing the cells in a medium containing a recombinant human IL-12 for the final 24 hours of culturing under the differentiation protocol.

59. (canceled)

60. A polypeptide comprising an extracellular polyethylene glycol (PEG) recognition element operably linked to an intracellular signaling domain, said polypeptide having at least 90%, 91%, 92%, 93%, 94%, 95%, 96%, 97%, 98%, 99% or 100% sequence identity to SEQ ID NOs: 178-186.


SEQUENCE LISTING (186 sequences)

SEQ ID NO: 1 - protein, 22 aa, Homo sapiens
MLLLVTSLLLCELPHPAFLLIP

SEQ ID NO: 2 - protein, 120 aa, synthetic construct
  REGION 1..120: note = FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, synthetic construct
  REGION 1..18: note = Whitlow Linker
GSTSGSGKPGSGEGSTKG

SEQ ID NO: 4 - protein, 107 aa, synthetic construct
  REGION 1..107: note = FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, synthetic construct
  REGION 1..245: note = FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, synthetic construct
  REGION 1..15: note = (GGGGS)3
GGGGSGGGGSGGGGS

SEQ ID NO: 26 - protein, 20 aa, synthetic construct
  REGION 1..20: note = Linker 3
GGSEGKSSGSGSESKSTGGS

SEQ ID NO: 27 - protein, 8 aa, synthetic construct
  REGION 1..8: note = Linker 4
GGGSGGGS

SEQ ID NO: 28 - protein, 12 aa, synthetic construct
  REGION 1..12: note = Linker 5
GGGSGGGSGGGS

SEQ ID NO: 29 - protein, 16 aa, synthetic construct
  REGION 1..16: note = Linker 6
GGGSGGGSGGGSGGGS

SEQ ID NO: 30 - protein, 20 aa, synthetic construct
  REGION 1..20: note = Linker 7
GGGSGGGSGGGSGGGSGGGS

SEQ ID NO: 31 - protein, 20 aa, synthetic construct
  REGION 1..20: note = Linker 8
GGGGSGGGGSGGGGSGGGGS

SEQ ID NO: 32 - protein, 25 aa, synthetic construct
  REGION 1..25: note = Linker 9
GGGGSGGGGSGGGGSGGGGSGGGGS

SEQ ID NO: 33 - protein, 14 aa, synthetic construct
  REGION 1..14: note = Linker 10
IRPRAIGGSKPRVA

SEQ ID NO: 34 - protein, 15 aa, synthetic construct
  REGION 1..15: note = Linker 11
GKGGSGKGGSGKGGS

SEQ ID NO: 35 - protein, 15 aa, synthetic construct
  REGION 1..15: note = Linker 12
GGKGSGGKGSGGKGS

SEQ ID NO: 36 - protein, 15 aa, synthetic construct
  REGION 1..15: note = Linker 13
GGGKSGGGKSGGGKS

SEQ ID NO: 37 - protein, 15 aa, synthetic construct
  REGION 1..15: note = Linker 14
GKGKSGKGKSGKGKS

SEQ ID NO: 38 - protein, 15 aa, synthetic construct
  REGION 1..15: note = Linker 15
GGGKSGGKGSGKGGS

SEQ ID NO: 39 - protein, 15 aa, synthetic construct
  REGION 1..15: note = Linker 16
GKPGSGKPGSGKPGS

SEQ ID NO: 40 - protein, 20 aa, synthetic construct
  REGION 1..20: note = Linker 17
GKPGSGKPGSGKPGSGKPGS

SEQ ID NO: 41 - protein, 20 aa, synthetic construct
  REGION 1..20: note = Linker 18
GKGKSGKGKSGKGKSGKGKS

SEQ ID NO: 42 - protein, 14 aa, synthetic construct
  REGION 1..14: note = Linker 19
STAGDTHLGGEDFD

SEQ ID NO: 43 - protein, 15 aa, synthetic construct
  REGION 1..15: note = Linker 20
GEGGSGEGGSGEGGS

SEQ ID NO: 44 - protein, 15 aa, synthetic construct
  REGION 1..15: note = Linker 21
GGEGSGGEGSGGEGS

SEQ ID NO: 45 - protein, 15 aa, synthetic construct
  REGION 1..15: note = Linker 22
GEGESGEGESGEGES

SEQ ID NO: 46 - protein, 15 aa, synthetic construct
  REGION 1..15: note = Linker 23
GGGESGGEGSGEGGS

SEQ ID NO: 47 - protein, 20 aa, synthetic construct
  REGION 1..20: note = Linker 24
GEGESGEGESGEGESGEGES

SEQ ID NO: 48 - protein, 18 aa, synthetic construct
  REGION 1..18: note = Linker 25
GSTSGSGKPGSGEGSTKG

SEQ ID NO: 49 - protein, 19 aa, synthetic construct
  REGION 1..19: note = Linker 26
PRGASKSGSASQTGSAPGS

SEQ ID NO: 50 - protein, 19 aa, synthetic construct
  REGION 1..19: note = Linker 27
GTAAAGAGAAGGAAAGAAG

SEQ ID NO: 51 - protein, 19 aa, synthetic construct
  REGION 1..19: note = Linker 28
GTSGSSGSGSGGSGSGGGG

SEQ ID NO: 52 - protein, 20 aa, synthetic construct
  REGION 1..20: note = Linker 29
GKPGSGKPGSGKPGSGKPGS

SEQ ID NO: 53 - protein, 4 aa, synthetic construct
  REGION 1..4: note = Linker 30
GSGS

SEQ ID NO: 54 - protein, 10 aa, synthetic construct
  REGION 1..10: note = Linker 31
APAPAPAPAP

SEQ ID NO: 55 - protein, 20 aa, synthetic construct
  REGION 1..20: note = Linker 32
APAPAPAPAPAPAPAPAPAP

SEQ ID NO: 56 - protein, 32 aa, synthetic construct
  REGION 1..32: note = Linker 33
AEAAAKEAAAKEAAAAKEAAAAKEAAAAKAAA

SEQ ID NO: 57 - protein, 15 aa, Homo sapiens
EPKSCDKTHTCPPCP

SEQ ID NO: 58 - protein, 12 aa, Homo sapiens
ERKCCVECPPCP

SEQ ID NO: 59 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 60 - protein, 12 aa, Homo sapiens
ESKYGPPCPSCP

SEQ ID NO: 61 - protein, 486 aa, synthetic construct
  REGION 1..486: note = anti-CD19 scFv CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1461 bp, synthetic construct
  misc_feature 1..1461: note = anti-CD19 scFv CAR
  source 1..1461: mol_type = other DNA
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagatga

SEQ ID NO: 63 - DNA, 1724 bp, synthetic construct
  misc_feature 1..1724: note = CAG Promoter
  source 1..1724: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 64 - DNA, 121 bp, synthetic construct
  misc_feature 1..121: note = SV40 terminator/polyadenylation
  source 1..121: mol_type = other DNA
aacttgtttattgcagcttataatggttacaaataaagcaatagcatcacaaatttcacaaataaagcatttttttcactgcattctagttgtggtttgtccaaactcatcaatgtatctt

SEQ ID NO: 65 - protein, 335 aa, Homo sapiens
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 66 - protein, 504 aa, synthetic construct
  REGION 1..504: note = HLA-G signal-Peptide-B2M-HLA-E
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 67 - DNA, 1515 bp, synthetic construct
  misc_feature 1..1515: note = HLA-G signal-Peptide-B2M-HLA-E
  source 1..1515: mol_type = other DNA
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 68 - protein, 312 aa, Homo sapiens
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 69 - protein, 471 aa, synthetic construct
  REGION 1..471: note = HLA-G signal-Peptide-B2M-HLA-G
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 70 - DNA, 1422 bp, synthetic construct
  misc_feature 1..1422: note = HLA-G signal-Peptide-B2M-HLA-G
  source 1..1422: mol_type = other DNA
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 71 - protein, 371 aa, synthetic construct
  REGION 1..371: note = truncated EGFR
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 72 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 73 - protein, 19 aa, synthetic construct
  REGION 1..19: note = P2A
ATNFSLLKQAGDVEENPGP

SEQ ID NO: 74 - protein, 556 aa, synthetic construct
  REGION 1..556: note = tEGFR-P2A-IL15
MRPSGTAGAALLALLAALCPASRAGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMSGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - DNA, 1668 bp, synthetic construct
  misc_feature 1..1668: note = tEGFR-P2A-IL15
  source 1..1668: mol_type = other DNA
atgaggccctcaggcactgccggggccgccctcctggccctgttagccgctttgtgtccagcaagccgcgccggagtgcggaaatgtaagaaatgcgaaggaccctgccggaaggtatgcaacggcattgggattggcgaattcaaggacagcctgagcattaatgctacaaacatcaagcactttaagaattgcaccagcattagcggcgatctgcatatactgccagtggctttccgaggcgactcttttactcatacccctccgctggaccctcaagagctggacattctcaagactgtgaaggaaattacggggtttctgctcattcaggcctggcctgaaaaccgcacggatttgcatgcctttgagaatctggaaataatcagaggccggacgaaacagcatggccagttcagcctcgcggtcgtctctttgaatattacgtcactcggcctcaggtccctcaaagagatttctgatggcgatgtcatcatctctggtaataagaatctgtgttacgcaaataccatcaattggaagaagctctttgggacctcaggtcaaaagactaaaattatctccaaccgcggcgagaacagctgtaaggctacaggccaggtttgccacgcgctctgctccccagagggttgctgggggcctgagccaagggattgcgtttcatgtcgcaacgtgtctcggggcagagaatgcgtggataaatgtaacctcttagagggcgaacctcgcgagtttgttgagaactcagaatgtatacagtgccaccccgaatgtcttcctcaggccatgaatatcacatgcaccggacgcggaccagacaactgtatccaatgtgctcactacattgacggacctcattgtgtgaaaacatgccccgcaggagttatgggagaaaacaacaccctcgtttggaaatatgccgatgcaggtcacgtatgtcacctgtgccacccaaactgcacttatgggtgcaccgggccgggcctggaggggtgccctacgaatggaccaaaaattcccagtattgcaactgggatggtcggggcactgttgttgctgcttgtggttgccctcgggataggcctgtttatgtctggctccggcgccaccaatttcagcctgctgaaacaggcaggcgacgtcgaagaaaatccaggaccaatgcgaatatcaaaaccacacttgcgcagcatttctatacagtgctatttgtgcttgttgctgaactctcacttcctcacagaggctgggatacacgttttcatacttggatgtttttcagctgggctgccgaagacagaggcgaattgggtgaatgtaatttcagacctcaagaagatcgaggatctcatccagtccatgcacatcgacgctactctgtacacagagagcgatgtccacccttcttgtaaggttaccgccatgaaatgcttccttttggaactccaagtcatctcattggaatcaggggatgcgtccattcatgacaccgtggaaaacctgataatactggctaacaacagcttgtcaagtaatgggaatgttactgagtccggttgtaaagaatgtgaagagctggaggagaagaacattaaggaatttttgcaatcttttgtacatattgttcagatgtttattaacacaagc

SEQ ID NO: 76 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 77 - protein, 19 aa, synthetic construct
  REGION 1..19: note = Signal Sequence
MEFGLSWVFLVALFRGVQC

SEQ ID NO: 78 - protein, 21 aa, synthetic construct
  REGION 1..21: note = Polivy epitope
ARSEDRYRNPKGSACSRIWQS

SEQ ID NO: 79 - protein, 287 aa, synthetic construct
  REGION 1..287: note = CD79b-P2A-IL15
MEFGLSWVFLVALFRGVQCARSEDRYRNPKGSACSRIWQSTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 80 - protein, 10 aa, synthetic construct
  REGION 1..10: note = CD20 mimitope
ACPYANPSLC

SEQ ID NO: 81 - protein, 296 aa, synthetic construct
  REGION 1..296: note = CD20 mimitope-P2A-IL15
MEFGLSWVFLVALFRGVQCACPYANPSLCGGGGSGGGGSACPYANPSLCTTTPAPRPPTPAPTIASQPLSLRPEACRPAAGGAVHTRGLDFACDIYIWAPLAGTCGVLLLSLVITATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 82 - protein, 176 aa, synthetic construct
  REGION 1..176: note = ErbB epitope
EGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGG

SEQ ID NO: 83 - protein, 376 aa, synthetic construct
  REGION 1..376: note = ErbB epitope-P2A-IL15
MEFGLSWVFLVALFRGVQCEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIGGGGSGGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 84 - DNA, 1000 bp, synthetic construct
  misc_feature 1..1000: note = CIITA targeting left homology arm
  source 1..1000: mol_type = other DNA
acaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcag

SEQ ID NO: 85 - DNA, 1000 bp, synthetic construct
  misc_feature 1..1000: note = CIITA targeting right homology arm
  source 1..1000: mol_type = other DNA
agccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaaggga

SEQ ID NO: 86 - DNA, 6604 bp, synthetic construct
  misc_feature 1..6604: note = CIITA targeting plasmid
  source 1..6604: mol_type = other DNA
cgcctgacctctcgaacaggaggtaaccatttaacaagaaagcagagtgatgttagattatagcaagatactgttgactgtagaaggctctgaggctagagagctgctttctataaaacagagtgatcatatattagaagaggtgttaaagacatgttcacaccaagctgagacttcctccttgataccaccaggaggatgggcagagactggaaaagacactaactttctccctatgggagtcagtattatttagcatcactttggcgggtcaccccaaaccatctgactacaagggtaccatatttgggttaacactcttttggtataatttatgttttagtccaatgtcttgggatgaaaatgacaggtgggccacttatgatctccagagaaattcagggcaatttggtgtgggagtaggcatggtagaggagagcagcatctaagaagtccccagcagaggctctcagcttgtcttgaggcatctgggcggagggctatgatactggccccatcctgcagaaggtggcagatattggcagctggcaccagtgcggttccattgtgatcatcatttctgaacgtcagactgttgaaggttcccccaacagactttctgtgcaactttctgtcttcaccaaattcagtccacagtaaggaagtgaaattaatttcagaggtgtggggagggcttaagggagtgtggtaaaattagagggtgttcagaaacagaaatctgaccgcttggggccaccttgcagggagagtttttttgatgatccctcacttgtttctttgcatgttggcttagcttggcgggctcccaactggtgactggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagtcgagaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgccgccaccatgaggccctcaggcactgccggggccgccctcctggccctgttagccgctttgtgtccagcaagccgcgccggagtgcggaaatgtaagaaatgcgaaggaccctgccggaaggtatgcaacggcattgggattggcgaattcaaggacagcctgagcattaatgctacaaacatcaagcactttaagaattgcaccagcattagcggcgatctgcatatactgccagtggctttccgaggcgactcttttactcatacccctccgctggaccctcaagagctggacattctcaagactgtgaaggaaattacggggtttctgctcattcaggcctggcctgaaaaccgcacggatttgcatgcctttgagaatctggaaataatcagaggccggacgaaacagcatggccagttcagcctcgcggtcgtctctttgaatattacgtcactcggcctcaggtccctcaaagagatttctgatggcgatgtcatcatctctggtaataagaatctgtgttacgcaaataccatcaattggaagaagctctttgggacctcaggtcaaaagactaaaattatctccaaccgcggcgagaacagctgtaaggctacaggccaggtttgccacgcgctctgctccccagagggttgctgggggcctgagccaagggattgcgtttcatgtcgcaacgtgtctcggggcagagaatgcgtggataaatgtaacctcttagagggcgaacctcgcgagtttgttgagaactcagaatgtatacagtgccaccccgaatgtcttcctcaggccatgaatatcacatgcaccggacgcggaccagacaactgtatccaatgtgctcactacattgacggacctcattgtgtgaaaacatgccccgcaggagttatgggagaaaacaacaccctcgtttggaaatatgccgatgcaggtcacgtatgtcacctgtgccacccaaactgcacttatgggtgcaccgggccgggcctggaggggtgccctacgaatggaccaaaaattcccagtattgcaactgggatggtcggggcactgttgttgctgcttgtggttgccctcgggataggcctgtttatgtctggctccggcgccaccaatttcagcctgctgaaacaggcaggcgacgtcgaagaaaatccaggaccaatgcgaatatcaaaaccacacttgcgcagcatttctatacagtgctatttgtgcttgttgctgaactctcacttcctcacagaggctgggatacacgttttcatacttggatgtttttcagctgggctgccgaagacagaggcgaattgggtgaatgtaatttcagacctcaagaagatcgaggatctcatccagtccatgcacatcgacgctactctgtacacagagagcgatgtccacccttcttgtaaggttaccgccatgaaatgcttccttttggaactccaagtcatctcattggaatcaggggatgcgtccattcatgacaccgtggaaaacctgataatactggctaacaacagcttgtcaagtaatgggaatgttactgagtccggttgtaaagaatgtgaagagctggaggagaagaacattaaggaatttttgcaatcttttgtacatattgttcagatgtttattaacacaagctgataaaacttgtttattgcagcttataatggttacaaataaagcaatagcatcacaaatttcacaaataaagcatttttttcactgcattctagttgtggtttgtccaaactcatcaatgtatcttatcatgtctgtaaggatcagccccaaggtaaaaaggccgggaaagcatcttaatttagcgtgcagtctcagctggtcctgccattccagataaacagagaaaccattctgaattggggatgggggtgaggatgggaacaggagtctgtgtcctgctggggcaggccattggaagatgtgaaagagttgtctatttccttccaccggagggagacttcaggtcagccaggtgtctggagtatgaaccatgtatcagcaccgaaaggttctagaagtcagactttcgggcagtgtgtcactaactctcagcatgctggcctggctcggcccacagcaaggtcttctcgcctccctttgggtaaatactgaggggtgcctctgcaggacgggacctctgccagactccactccatacccagagaagcagggaaaccaaaattggagtcagccttgaggtgtagctgttgagccctcagcagctggggagagctggcggatgctgccctccccccagtttcctaatggtgttgtttaaaaagggtcaggggacgggggaacagatggtgggaagagcacagtgcagacacctggcaccggctctgaaggcagcatggcagctacaccgttggctgggaagggtgtgcccctgaagaagtcgtttacattctcgagtcaattttcctggagtgtacaatggacctgtgggaaagcctgtatgaaagggtaatgatgagggacctagcacagtgtccaatattttataggaactggaattgagctcataggagctcaattttattggcattgctgttgttggatggttaaaggggtggtatcccttttctcagactcccctgaaatgtatggtttgctttgaacccagagactgatgacaggtctgccggtgtggttgggtgcagccttaagttgctacgggaaagtgttggagggggagaagtcagaggtaaccttgccccctccctcaattccagatgaggaaattcaggcctgaaaagggagatccaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattc

SEQ ID NO: 87 - DNA, 1042 bp, synthetic construct
  misc_feature 1..1042: note = B2M targeting left homology arm
  source 1..1042: mol_type = other DNA
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 88 - DNA, 1023 bp, synthetic construct
  misc_feature 1..1023: note = B2M targeting right homology arm
  source 1..1023: mol_type = other DNA
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 89 - DNA, 7813 bp, synthetic construct
  misc_feature 1..7813: note = B2M Targeting Plasmid
  source 1..7813: mol_type = other DNA
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcggcctccaacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 90 - DNA, 1205 bp, synthetic construct
  misc_feature 1..1205: note = AAVS1 targeting left homology arm
  source 1..1205: mol_type = other DNA
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 91 - DNA, 1200 bp, synthetic construct
  misc_feature 1..1200: note = AAVS1 targeting right homology arm
  source 1..1200: mol_type = other DNA
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 92 - DNA, 7971 bp, synthetic construct
  misc_feature 1..7971: note = AAVS1 Targeting plasmid
  source 1..7971: mol_type = other DNA
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctgcggccgcacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcacgacgactcctgctccaaggcctcctacacctgcaccaaccattgcaagtcagccgttgagcctccggccagaagcatgtcgcccagccgcaggcggggctgtacacacgagaggcttggatttcgcatgtgacatctatatctgggccccactggccggcacctgcggcgtgctgctgctgagcctggtgatcaccaagcgaggccgcaaaaaactcctttatatattcaagcaaccttttatgaggcccgtccagaccacgcaagaggaagatgggtgctcttgccgctttccagaggaagaggaggggggctgcgaacttagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataataaaacttgtttattgcagcttataatggttacaaataaagcaatagcatcacaaatttcacaaataaagcatttttttcactgcattctagttgtggtttgtccaaactcatcaatgtatcttaccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 93 - DNA, 24 bp, synthetic construct
  misc_feature 1..24: note = B2M target gRNA
  source 1..24: mol_type = other DNA
tttactcacgtcatccagcagaga

SEQ ID NO: 94 - DNA, 24 bp, synthetic construct
  misc_feature 1..24: note = AAVS1 target gRNA
  source 1..24: mol_type = other DNA
tttatctgtcccctccaccccaca

SEQ ID NO: 95 - DNA, 24 bp, synthetic construct
  misc_feature 1..24: note = CIITA target gRNA
  source 1..24: mol_type = other DNA
tttaccttggggctctgacaggta

SEQ ID NO: 96 - protein, 128 aa, synthetic construct
  REGION 1..128: note = CD22_D04
QVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTVIRYETWGQGTQVTVSS

SEQ ID NO: 97 - protein, 122 aa, synthetic construct
  REGION 1..122: note = CD22_CNTY_VHH1_A01
EVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSS

SEQ ID NO: 98 - protein, 120 aa, synthetic construct
  REGION 1..120: note = CD22_CNTY_VHH1_E04
EVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSS

SEQ ID NO: 99 - DNA, 384 bp, synthetic construct
  misc_feature 1..384: note = CD22_D04
  source 1..384: mol_type = other DNA
caggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagc

SEQ ID NO: 100 - DNA, 366 bp, synthetic construct
  misc_feature 1..366: note = CD22_CNTY_VHH1_A01
  source 1..366: mol_type = other DNA
gaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagc

SEQ ID NO: 101 - DNA, 360 bp, synthetic construct
  misc_feature 1..360: note = CD22_CNTY_VHH1_E04
  source 1..360: mol_type = other DNA
gaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagc

SEQ ID NO: 102 - protein, 119 aa, synthetic construct
  REGION 1..119: note = IgG4 CH3 Hinge
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 21 aa, synthetic construct
  REGION 1..21: note = IgK Signal Peptide Variant
MARSPAQLLGLLLLWLSGARC

SEQ ID NO: 104 - protein, 486 aa, synthetic construct; identical to SEQ ID NO: 61
  REGION 1..486: note = FMC63_CD28_CD28_CD28_CD3Z (P1209)

SEQ ID NO: 105 - protein, 443 aa, synthetic construct
  REGION 1..443: note = A01_Ig4 CH3_CD28_41BB_CD3Z (P1362)
MARSPAQLLGLLLLWLSGARCEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 502 aa, synthetic construct
  REGION 1..502: note = A01-A01_CD28_CD3Z (P1631)
MARSPAQLLGLLLLWLSGARCEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSGSTSGSGKPGSGEGSTKGEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 375 aa, synthetic construct
  REGION 1..375: note = D04AMD11_CD8_CD28_41BB_CD3Z (P1729)
MARSPAQLLGLLLLWLSGARCQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 498 aa, synthetic construct
  REGION 1..498: note = E04-E04_CD28_CD28_CD28_CD3Z (P1633)
MARSPAQLLGLLLLWLSGARCEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - protein, 506 aa, synthetic construct
  REGION 1..506: note = D04AMD11-E04_CD28_CD28_CD28_CD3Z (P1702)
MARSPAQLLGLLLLWLSGARCQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 110 - protein, 336 aa, synthetic construct
  REGION 1..336: note = E04_Ig4 _CD28_41BB_CD3Z (P1356)
MARSPAQLLGLLLLWLSGARCEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSESKYGPPCPPCPGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - protein, 515 aa, synthetic construct
  REGION 1..515: note = D04AMD11-A01_CD8_CD28_41BB_CD3Z (P1734)
MARSPAQLLGLLLLWLSGARCQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGSTSGSGKPGSGEGSTKGEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - DNA, 1458 bp, synthetic construct
  misc_feature 1..1458: note = FMC63_CD28_CD28_CD28_CD3Z (P1209)
  source 1..1458: mol_type = other DNA
atgctgctgctggttacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 113 - DNA, 1329 bp, synthetic construct
  misc_feature 1..1329: note = A01_Ig4 CH3_CD28_41BB_CD3Z (P1362)
  source 1..1329: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcgagtccaaatacggtccgccatgcccaccatgcccagggcagccccgagagccacaggtgtacaccctgcccccatcccaagaggagatgaccaagaaccaagtcagcctgacctgcctggtcaaaggcttctaccccagcgacatcgccgtggagtgggagagcaatgggcagccggagaacaactacaagaccacgcctcccgtgctggactccgacggctccttcttcctctactcccggctcaccgtggacaagagcaggtggcaggaggggaatgtgttctcatgctccgtgatgcatgaggctctgcacaaccactacacacagaagagcctctccctgtctctgggaaagttttgggtgctggtggtggttggtggagtcctggcttgctattccttgctagtaacagtggcctttattattttctgggtgaaacgcggccgcaagaaactcctgtatatattcaaacaaccatttatgaggccagtacaaactactcaagaggaagatggctgtagctgccgatttccagaagaagaagaaggaggatgtgagctcagagtgaagttcagcaggagcgcagacgcccccgcgtaccagcagggccagaaccagctctataacgaactcaatctaggacgaagagaggagtacgatgttttggacaagcggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccaggggctcagtacagccaccaaggacacctacgacgcccttcacatgcaggccctgccccctcgc

SEQ ID NO: 114 - DNA, 1509 bp, synthetic construct
  misc_feature 1..1509: note = A01-A01_CD28_CD3Z (P1631)
  source 1..1509: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcggctctacaagcggcagcggcaaacctggatctggcgagggatctaccaagggcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataa

SEQ ID NO: 115 - DNA, 1125 bp, synthetic construct
  misc_feature 1..1125: note = D04AMD11_CD8_CD28_41BB_CD3Z (P1729)
  source 1..1125: mol_type = other DNA
atggccagatctcctgctcaactgctgggactgctgctgctgtggcttagcggagccagatgccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 116 - DNA, 1494 bp, synthetic construct
  misc_feature 1..1494: note = E04-E04_CD28_CD28_CD28_CD3Z (P1633)
  source 1..1494: mol_type = other DNA
atggccagatctcctgctcaactgctgggactgctgctgctgtggcttagcggagccagatgcgaggtgcagctgttggagagcggcgggggacttgttcaacccggaggctctcttaggttatcttgcgcagctagtggatttacgagctccagttacgtgatgggatggtatcgacaggctcctggaaaagaaagagagttcgtgagctctattagcaccggcggcgatgcgtattacgcagattcagtgaaaggccgattcaccatttccagggataactccaaaaacactctctacctgcaaatgaacagcctgagagccgaagacaccgctgtttattattgcgccgccgacgtttggtattaccacggaggcgcttatgattattggggccaggggactcaggtgacggtctcatctggctctacaagcggcagcggcaaacctggatctggcgagggatctaccaagggcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 117 - DNA, 1518 bp, synthetic construct
  misc_feature 1..1518: note = D04AMD11-E04_CD28_CD28_CD28_CD3Z (P1702)
  source 1..1518: mol_type = other DNA
atggccagatctcctgctcaactgctgggactgctgctgctgtggcttagcggagccagatgccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcgggtctacctcagggtcagggaaacccggaagcggcgaagggtctacaaaaggtgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 118 - DNA, 1008 bp, synthetic construct
  misc_feature 1..1008: note = E04_Ig4 _CD28_41BB_CD3Z (P1356)
  source 1..1008: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcgagtccaaatacggtccgccatgcccaccatgcccaggaaagttttgggtgctggtggtggttggtggagtcctggcttgctattccttgctagtaacagtggcctttattattttctgggtgaaacgcggccgcaagaaactcctgtatatattcaaacaaccatttatgaggccagtacaaactactcaagaggaagatggctgtagctgccgatttccagaagaagaagaaggaggatgtgagctcagagtgaagttcagcaggagcgcagacgcccccgcgtaccagcagggccagaaccagctctataacgaactcaatctaggacgaagagaggagtacgatgttttggacaagcggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccaggggctcagtacagccaccaaggacacctacgacgcccttcacatgcaggccctgccccctcgc

SEQ ID NO: 119 - DNA, 1545 bp, synthetic construct
  misc_feature 1..1545: note = D04AMD11-A01_CD8_CD28_41BB_CD3Z (P1734)
  source 1..1545: mol_type = other DNA
atggccagatctcctgctcaactgctgggactgctgctgctgtggcttagcggagccagatgccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcgggtctacctcagggtcagggaaacccggaagcggcgaagggtctacaaaaggtgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcacaacaactccagccccaagaccacctacgcctgcacctactatcgcatctcaaccactgtccctgcgccctgaggcatgccgaccagcagccggtggcgcggtgcatacccgcggactggactttgcctgcgatttttgggtgctggtggtggttggtggagtcctggcttgctatagcttgctagtaacagtggcctttattattttctgggtgaaacggggcagaaagaaactcctgtatatattcaaacaaccatttatgcgaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 120 - protein, 623 aa, synthetic construct
  REGION 1..623: note = FMC63LC_E04_FMC63HC_CD28_CD28_CD28_CD3Z (P1973)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 625 aa, synthetic construct
  REGION 1..625: note = FMC63LC_A01_FMC63HC_CD28_CD28_CD28_CD3Z (P1988)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 748 aa, synthetic construct
  REGION 1..748: note = FMC63LC_E04_E04_FMC63HC_CD28_CD28_CD28_CD3Z (P1974)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGGGGSEVQLLESGGGLVQPGGSLRLSCAASGFTSSCYVMGWYRQAPGKEREFVCTISTGGDAYYADSVKGRFTITRDNSKNTLYLQMNSLRAEDTAVYYCAADVWYYHGGAYDYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 750 aa, synthetic construct
  REGION 1..750: note = FMC63LC_E04_A01_FMC63HC_CD28_CD28_CD28_CD3Z (P2012)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGGGGSEVQLLESGGGLVQPGGTLRLSCAASGLTCYSYAMGWYRQAPGKEREFVSAISSGGSAYYADSVKGRFTICRDNSKNTLYLQMNSLRAEDTAVYYCAVGPYYGFRAVTEADYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 756 aa, synthetic construct
  REGION 1..756: note = FMC63LC_E04_D04AMD11_FMC63HC_CD28_CD28_CD28_CD3Z (P2013)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGGGGSQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 623 aa, synthetic construct
  REGION 1..623: note = E04_FMC63_CD28_CD28_CD28_CD3Z (P1972)
MARSPAQLLGLLLLWLSGARCEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 625 aa, synthetic construct
  REGION 1..625: note = A01_FMC63_CD28_CD28_CD28_CD3Z (P2014)
MARSPAQLLGLLLLWLSGARCEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 756 aa, synthetic construct
  REGION 1..756: note = D04AMD11_E04_FMC63_CD28_CD28_CD28_CD3Z (P2015)
MARSPAQLLGLLLLWLSGARCQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGGGGSEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 1016 aa, synthetic construct
  REGION 1..1016: note = D04_E04_A01_D04_FMC63_CD28_CD28_CD28_CD3Z (P2016)
MARSPAQLLGLLLLWLSGARCQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGGGGSEVQLLESGGGLVQPGGSLRLSCAASGFTSSSYVMGWYRQAPGKEREFVSSISTGGDAYYADSVKGRFTISRDNSKNTLYLQMNSLRAEDTAVYYCAADVWYYHGGAYDYWGQGTQVTVSSGGGGSEVQLLESGGGLVQPGGTLRLSCAASGLTCYSYAMGWYRQAPGKEREFVSAISSGGSAYYADSVKGRFTICRDNSKNTLYLQMNSLRAEDTAVYYCAVGPYYGFRAVTEADYWGQGTQVTVSSGGGGSQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 752 aa, synthetic construct
  REGION 1..752: note = FMC63LC_A01_A01_FMC63HC_CD28_CD28_CD28_CD3Z (P2193)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSGGGGSEVQLLESGGGLVQPGGTLRLSCAASGLTCYSYAMGWYRQAPGKEREFVSAISSGGSAYYADSVKGRFTICRDNSKNTLYLQMNSLRAEDTAVYYCAVGPYYGFRAVTEADYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 758 aa, synthetic construct
  REGION 1..758: note = FMC63LC_D04AMD11_A01_FMC63HC_CD28_CD28_CD28_CD3Z (P2191)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGGGGSEVQLLESGGGLVQPGGTLRLSCAASGLTCYSYAMGWYRQAPGKEREFVSAISSGGSAYYADSVKGRFTICRDNSKNTLYLQMNSLRAEDTAVYYCAVGPYYGFRAVTEADYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 885 aa, synthetic construct
  REGION 1..885: note = FMC63LC_A01_D04AMD11__A01_FMC63HC_CD28_CD28_CD28_CD 3Z (P2195)
MARSPAQLLGLLLLWLSGARCDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVQLLESGGGLVQPGGSLRLSCAASGLTSYSYAMGWYRQAPGKEREFVSAISSGGSAYYADSVKGRFTISRDNSKNTLYLQMNSLRAEDTAVYYCAVGPYYGFRAVTEADYWGQGTQVTVSSGGGGSQVQLVESGGGLVQAGGSLRLSCAASGSEFTGYPMGWFRQAPGKEREFVAGSVGIGGSTNYADSVKGRFTISRDNAKNTVYLQMNSLKPEDTAVYYCAADKDYYKPYSRYRTAIRYDTWGQGTQVTVSSGGGGSEVQLLESGGGLVQPGGTLRLSCAASGLTCYSYAMGWYRQAPGKEREFVSAISSGGSAYYADSVKGRFTICRDNSKNTLYLQMNSLRAEDTAVYYCAVGPYYGFRAVTEADYWGQGTQVTVSSGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - DNA, 1869 bp, synthetic construct
  misc_feature 1..1869: note = FMC63LC_E04_FMC63HC_CD28_CD28_CD28_CD3Z (P1973)
  source 1..1869: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 133 - DNA, 1875 bp, synthetic construct
  misc_feature 1..1875: note = FMC63LC_A01_FMC63HC_CD28_CD28_CD28_CD3Z (P1988)
  source 1..1875: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 134 - DNA, 2244 bp, synthetic construct
  misc_feature 1..2244: note = FMC63LC_E04_E04_FMC63HC_CD28_CD28_CD28_CD3Z (P1974)
  source 1..2244: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcggtggcggatcagaagtccagctgctggaaagcggtggcggtctggtccagcccggcggcagcctgcgcctgtcctgtgccgctagcggtttcacttccagctgctatgtcatgggttggtaccgccaggcccccggtaaggagcgcgaattcgtgtgcaccatttccactggcggcgacgcttattatgctgatagcgtgaagggtcgcttcactattacccgcgacaacagcaaaaacactctgtatctgcagatgaactccctgcgcgctgaggataccgccgtctactactgcgctgccgatgtgtggtattatcatggtggtgcctatgactactggggtcagggcactcaggtcaccgtcagctccggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 135 - DNA, 2250 bp, synthetic construct
  misc_feature 1..2250: note = FMC63LC_E04_A01_FMC63HC_CD28_CD28_CD28_CD3Z (P2012)
  source 1..2250: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcggtggcggatcagaagtccagctgctggaaagcggtggcggtctggtccagcctggcggcaccctgcgcctgtcctgtgccgctagcggcctgacctgctatagctatgccatgggttggtaccgccaggcccctggtaaggagcgcgaattcgtgtccgctatttccagcggcggctccgcctattatgctgatagcgtcaagggtcgcttcaccatttgccgcgacaacagcaaaaacactctgtatctgcagatgaactccctgcgcgctgaggataccgccgtctactactgcgctgtgggcccttattatggcttccgcgctgtgactgaggctgactactggggtcagggcactcaggtgactgtgagcagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 136 - DNA, 2268 bp, synthetic construct
  misc_feature 1..2268: note = FMC63LC_E04_D04AMD11_FMC63HC_CD28_CD28_CD28_CD3Z (P2013)
  source 1..2268: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcggtggcggatcacaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 137 - DNA, 1869 bp, synthetic construct
  misc_feature 1..1869: note = E04_FMC63_CD28_CD28_CD28_CD3Z (P1972)
  source 1..1869: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcagtacttctggtagcggaaaacccggtagcggcgaggggtcaactaaaggagacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcaggagcgcagacgcccccgcgtaccagcagggccagaaccagctctataacgagctcaatctaggacgaagagaggagtacgatgttttggacaagcgacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggactcagtacagccaccaaggacacctacgacgcccttcacatgcaggccctgccccctcgc

SEQ ID NO: 138 - DNA, 1875 bp, synthetic construct
  misc_feature 1..1875: note = A01_FMC63_CD28_CD28_CD28_CD3Z (P2014)
  source 1..1875: mol_type = other DNA
atggccagatctcctgctcaactgctgggactgctgctgctgtggcttagcggagccagatgcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcggcagtacttctggtagcggaaaacccggtagcggcgaggggtcaactaaaggagacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 139 - DNA, 2268 bp, synthetic construct
  misc_feature 1..2268: note = D04AMD11_E04_FMC63_CD28_CD28_CD28_CD3Z (P2015)
  source 1..2268: mol_type = other DNA
atggccagatctcctgctcaactgctgggactgctgctgctgtggcttagcggagccagatgccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcggcggtggcggatcagaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggcagtacttctggtagcggaaaacccggtagcggcgaggggtcaactaaaggagacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 140 - DNA, 3048 bp, synthetic construct
  misc_feature 1..3048: note = D04_E04_A01_D04_FMC63_CD28_CD28_CD28_CD3Z (P2016)
  source 1..3048: mol_type = other DNA
atggccagatctcctgctcaactgctgggactgctgctgctgtggcttagcggagccagatgccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagtggcggtggcggatcagaggtacaacttttggagtcaggcggtggactggtacaaccgggtggttcattgcgtttgagctgcgctgcctctggttttaccagctcctcctacgtgatgggctggtatcgccaagcgccgggcaaagaacgcgagtttgtcagctcgatcagcaccggtggtgatgcctactacgcggactcggtaaaaggccgttttacgatcagtcgtgataattccaagaataccttgtacctgcaaatgaatagccttcgcgcagaagacacagcggtgtattattgtgccgctgacgtttggtactaccacggcggcgcgtacgattattggggccagggtacccaggtgacggtctcgagcggtggcggtggttctgaagtccagctgctggaaagcggtggcggtctggtccagcctggcggcaccctgcgcctgtcctgtgccgctagcggcctgacctgctatagctatgccatgggttggtaccgccaggcccctggtaaggagcgcgaattcgtgtccgctatttccagcggcggctccgcctattatgctgatagcgtcaagggtcgcttcaccatttgccgcgacaacagcaaaaacactctgtatctgcagatgaactccctgcgcgctgaggataccgccgtctactactgcgctgtgggcccttattatggcttccgcgctgtgactgaggctgactactggggtcagggcactcaggtgactgtgagcagcggtggtggcggatctcaggtccagctggtggaaagcggcggtggtctggtgcaggctggcggtagcctgcgcctgagctgcgctgccagcggttccgagtttactggctaccctatgggttggttccgccaggcccccggtaaagagcgcgaattcgtggccggtagcgtcggcattggcggctccactaattacgctgatagcgtcaaaggtcgctttactattagccgcgataacgccaaaaatactgtgtacctgcagatgaattccctgaaacccgaagataccgccgtctactattgcgccgctgataaggattattataagccctactcccgctaccgcactgccattcgctatgacacttggggtcagggcactcaggtgactgtgagctccggcagtacttctggtagcggaaaacccggtagcggcgaggggtcaactaaaggagacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 141 - DNA, 2256 bp, synthetic construct
  misc_feature 1..2256: note = FMC63LC_A01_A01_FMC63HC_CD28_CD28_CD28_CD3Z (P2193)
  source 1..2256: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcggcggtggcggatcagaagtccagctgctggaaagcggtggcggtctggtccagcctggcggcaccctgcgcctgtcctgtgccgctagcggcctgacctgctatagctatgccatgggttggtaccgccaggcccctggtaaggagcgcgaattcgtgtccgctatttccagcggcggctccgcctattatgctgatagcgtcaagggtcgcttcaccatttgccgcgacaacagcaaaaacactctgtatctgcagatgaactccctgcgcgctgaggataccgccgtctactactgcgctgtgggcccttattatggcttccgcgctgtgactgaggctgactactggggtcagggcactcaggtgactgtgagcagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 142 - DNA, 2274 bp, synthetic construct
  misc_feature 1..2274: note = FMC63LC_D04AMD11_A01_FMC63HC_CD28_CD28_CD28_CD3Z (P2191)
  source 1..2274: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggccaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcggcggtggcggatcagaagtccagctgctggaaagcggtggcggtctggtccagcctggcggcaccctgcgcctgtcctgtgccgctagcggcctgacctgctatagctatgccatgggttggtaccgccaggcccctggtaaggagcgcgaattcgtgtccgctatttccagcggcggctccgcctattatgctgatagcgtcaagggtcgcttcaccatttgccgcgacaacagcaaaaacactctgtatctgcagatgaactccctgcgcgctgaggataccgccgtctactactgcgctgtgggcccttattatggcttccgcgctgtgactgaggctgactactggggtcagggcactcaggtgactgtgagcagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 143 - DNA, 2655 bp, synthetic construct
  misc_feature 1..2655: note = FMC63LC_A01_D04AMD11__A01_FMC63HC_CD28_CD28_CD28_CD 3Z (P2195)
  source 1..2655: mol_type = other DNA
atggccagatctcctgctcaactgctgggactgctgctgctgtggcttagcggagccagatgcgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaggtacaacttttggagtcaggcggtggactggtacaaccgggtggttcattgcgtttgagctgcgctgcctctggtttgacctcttattcctacgcgatgggctggtatcgccaagcgccgggcaaagaacgcgagtttgtcagcgcaatcagctcgggtggtagcgcgtactacgcggactcggtaaaaggccgttttacgatcagtcgtgataattccaagaataccttgtacctgcaaatgaatagccttcgcgcagaagacacagcggtgtattattgtgccgttggaccgtactacggatttagagcggttaccgaagcagattattggggccagggtacccaggtgacggtctcgagcggcggtggcggatcacaggtgcagctggttgagtctgggggaggccttgtccaggctggggggtccctgagactctcctgtgcagcgtctggaagcgaattcaccggttatcccatgggctggtttcgccaggctccaggcaaggaaagggagtttgtcgctggctccgtaggtatcggtggtagtacaaactatgcagactccgtgaagggccgattcaccatctccagagacaatgcgaagaacacggtctatctgcaaatgaacagcctgaagccagaggacacggctgtgtattactgtgcggccgacaaagactactacaaaccttatagtcgatataggaccgctatcaggtacgatacctggggccaagggacccaggtcaccgtctcgagcggtggcggtggttctgaagtccagctgctggaaagcggtggcggtctggtccagcctggcggcaccctgcgcctgtcctgtgccgctagcggcctgacctgctatagctatgccatgggttggtaccgccaggcccctggtaaggagcgcgaattcgtgtccgctatttccagcggcggctccgcctattatgctgatagcgtcaagggtcgcttcaccatttgccgcgacaacagcaaaaacactctgtatctgcagatgaactccctgcgcgctgaggataccgccgtctactactgcgctgtgggcccttattatggcttccgcgctgtgactgaggctgactactggggtcagggcactcaggtgactgtgagcagcggcagtacttctggtagcggaaaacccggtagcggcgaggggtcaactaaaggagaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttatagcctgcttgtgaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 144 - DNA, 63 bp, synthetic construct
  misc_feature 1..63: note = IgK Signal Peptide Variant
  source 1..63: mol_type = other DNA
atggccagatctcctgctcaactgctgggactgctgctgctgtggcttagcggagccagatgc

SEQ ID NO: 145 - protein, 16 aa, synthetic construct
  REGION 1..16: note = CD33 Signal Peptide
MPLLLLLPLLWAGALA

SEQ ID NO: 146 - DNA, 48 bp, synthetic construct
  misc_feature 1..48: note = CD33 Signal Peptide
  source 1..48: mol_type = other DNA
atgcctttgctgcttcttctgcccctgctttgggctggcgccctggca

SEQ ID NO: 147 - protein, 248 aa, synthetic construct
  REGION 1..248: note = Anti-PEG scFv
EVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAGSTSGSGKPGSGEGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELK

SEQ ID NO: 148 - DNA, 744 bp, synthetic construct
  misc_feature 1..744: note = Anti-PEG scFv
  source 1..744: mol_type = other DNA
gaagtgaagctggttgagagcggcggaggactggtgcagcctggcggaagcctgaaactgtcttgcgccaccagcggcttcacctttagcgactactacatctactgggtgcggcagacccctgagaagcggctggaatgggtcgcctctatcagcaacggaggcggcagcacatactatcctgataccctgaaaggcagatttaccatcagccgggacagcgccaagaacacactgtacctgcagatgagcagactgaaaagcgaggatacagccatgtactactgcgccagacagcacgacagcagctacctggcctggttcgcctactggggccagggcaccctggtgaccgtgtctgccggcagcaccagcggatctggcaagcccggctctggagagggctctgatgtgctgatgacccagacacctctgagcctgcctgtgtccctgggcgaccaggccagcattagctgcagatccagccagagcatcgtgcacagcaatggcaacacctacctggaatggtacctgcaaaagcctggccaatctccaaagctgcttatctacaaggtgtccaaccggttcagcggcgtgcccgacagattcagcggctccggctccggcacagacttcaccctgaagatcagtagagtggaagccgaggacctgggagtgtactattgcttccagggctctcacgtgccacctaccttcggtgctggcacaaagctcgagctgaag

SEQ ID NO: 149 - protein, 248 aa, synthetic construct
  REGION 1..248: note = Anti-PEG scFv
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKGSTSGSGKPGSGEGSEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSA

SEQ ID NO: 150 - DNA, 744 bp, synthetic construct
  misc_feature 1..744: note = Anti-PEG scFv
  source 1..744: mol_type = other DNA
gacgtgctgatgacccagacacctctgagcctgcctgtgtccctgggcgaccaggccagcatcagctgtagaagcagccagagcatcgtgcacagcaacggcaacacctacctggaatggtacctgcaaaagcctggccaaagccctaagcttctgatctacaaggtgtccaacagattcagcggagttccagacagatttagcggcagcggttccggcaccgacttcaccctgaaaatctctagagtggaagccgaggatctgggcgtgtactactgcttccagggcagccacgtgccccccaccttcggcgctggcacaaagctcgagctgaaaggctctacatccggctccggcaagcccggcagcggcgagggctctgaggtgaagctggtggaaagcggcggcggcctggtccagcctggaggatctctgaagctgtcctgcgctacaagcggattcacctttagcgactactacatctactgggtgcggcagacccctgagaagcggctggaatgggtcgcctctatttctaatggcggcggaagcacatactatcctgataccctgaagggcagattcaccatcagccgcgacagcgccaagaacacactgtacctgcagatgagccggctgaaaagcgaggacaccgccatgtactattgcgccagacagcacgattctagctacctggcctggttcgcctactggggccagggcaccctggtgaccgtgtctgct

SEQ ID NO: 151 - protein, 241 aa, synthetic construct
  REGION 1..241: note = Anti-PEG scFv
QIVLTQSPAIMSAFPGERVTLTCSASSSVRSSYLCWYQQKPGSSPKLWIYSTSNLASGVPARFSGSGSGTSYSLTISSMEAEDAASYFCHQWSSYPRTFGGGTKLEIKGSTSGSGKPGSGEGSEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSS

SEQ ID NO: 152 - DNA, 723 bp, synthetic construct
  misc_feature 1..723: note = Anti-PEG scFv
  source 1..723: mol_type = other DNA
cagatcgtgctgacccagagcccagcaatcatgtccgccttccctggcgaacgggtgacactgacatgcagcgccagctctagcgtgcggagcagctatctgtgttggtaccaacagaaacctggcagcagccctaagctgtggatctacagtacctccaatctggcctctggagtgcccgctagattcagcggatctggctccggcaccagctacagcctgaccattagcagcatggaagccgaggatgccgccagctacttttgccaccagtggagctcttaccccagaacattcggcggcggcacaaagctggaaatcaagggcagcacaagcggctcaggcaagcccggcagcggcgagggcagcgaggtgaagctggaggaaagcggcggcggcctggtgcaacctggaggaagcatgaaactgagctgtgccgctagcggatttatcttctctgatgcttggatggactgggttcgccagtcccctgagagaggcctcgaatgggtggccgagatcagatccaaggccaacggcctggccccttactacgccgagagcgtgaagggtagattcaccatcagccgggacgacagcaagtcttctgtctacctgcaaatgaacaacctgagaagcgaggacaccggcatctactactgcaccagcaccctgtactacttcgactattggggacagggcaccaccctgacagtgtcctcc

SEQ ID NO: 153 - protein, 241 aa, synthetic construct
  REGION 1..241: note = Anti-PEG scFv
QIVLTQSPAIMSAFPGERVTLTCSASSSVRSSYLAWYQQKPGSSPKLWIYSTSNLASGVPARFSGSGSGTSYSLTISSMEAEDAASYFCHQWSSYPRTFGGGTKLEIKGSTSGSGKPGSGEGSEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSS

SEQ ID NO: 154 - DNA, 723 bp, synthetic construct
  misc_feature 1..723: note = Anti-PEG scFv
  source 1..723: mol_type = other DNA
cagatcgttctgacacagtccccagctattatgagcgccttccccggagagcgggtgacactgacctgtagcgcctcttccagcgtgcggagcagctatctggcctggtaccagcagaagcctggtagcagtcccaagctgtggatctacagcaccagcaacctggcctccggagtgcccgccaggttcagcggctccggcagcggcacaagctatagcctgacaatcagctccatggaagccgaggacgctgcctcttacttctgccaccagtggagctcttaccctagaaccttcggcggcggcaccaagctggaaatcaagggctctacaagcggcagcggaaaacctggcagcggcgagggaagcgaggtgaagctggaagagagcggaggaggccttgtgcagcctggcggcagcatgaagctcagctgcgccgcttcaggcttcatcttttctgatgcctggatggactgggtcagacagtcccctgagagaggcctggaatgggtggccgagatcagaagcaaggccaatggcctggctccatactacgccgaatctgtgaaaggcagatttaccatctctcgggacgacagcaagagcagcgtgtacctgcaaatgaacaacctgagatctgaggatacaggcatctactactgcaccagcaccctgtactacttcgactactggggccaaggcaccaccctgaccgtgtcctct

SEQ ID NO: 155 - protein, 241 aa, synthetic construct
  REGION 1..241: note = Anti-PEG scFv
EVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSGSTSGSGKPGSGEGSQIVLTQSPAIMSAFPGERVTLTCSASSSVRSSYLCWYQQKPGSSPKLWIYSTSNLASGVPARFSGSGSGTSYSLTISSMEAEDAASYFCHQWSSYPRTFGGGTKLEIK

SEQ ID NO: 156 - DNA, 723 bp, synthetic construct
  misc_feature 1..723: note = Anti-PEG scFv
  source 1..723: mol_type = other DNA
gaggtgaagctggaagagagcggcggcggcctggtgcaacctggcggcagcatgaagctgtcatgcgccgcttctggatttatcttcagcgacgcctggatggactgggtgcggcagagccctgagcggggcctggaatgggtcgccgagattagaagcaaggccaatggcctcgccccttactacgccgaaagcgtgaaaggcagattcacaatctcaagagatgacagcaagagcagcgtgtacctgcagatgaacaacctgcggagcgaggataccggcatctactattgtacctctacactgtactacttcgactactggggccagggcacaaccctgaccgtgtcctctggatccaccagcggcagcggaaaacctggcagcggagagggcagccagatcgtgctgacacagtcccccgctatcatgagcgccttccccggcgagagagtgaccctgacctgtagcgcctcttctagtgttagaagcagttacctgtgctggtaccagcaaaagcctggctcttctccaaagctgtggatctacagcaccagcaacctggctagcggcgtgcctgctaggtttagcggatccggcagcggcaccagctacagcctgaccatcagcagcatggaagccgaggacgccgccagctatttctgccaccagtggtccagctaccccagaacattcggcggcggaaccaagctggaaatcaag

SEQ ID NO: 157 - protein, 241 aa, synthetic construct
  REGION 1..241: note = Anti-PEG scFv
EVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSGSTSGSGKPGSGEGSQIVLTQSPAIMSAFPGERVTLTCSASSSVRSSYLAWYQQKPGSSPKLWIYSTSNLASGVPARFSGSGSGTSYSLTISSMEAEDAASYFCHQWSSYPRTFGGGTKLEIK

SEQ ID NO: 158 - DNA, 723 bp, synthetic construct
  misc_feature 1..723: note = Anti-PEG scFv
  source 1..723: mol_type = other DNA
gaagtgaagctggaagagagcggaggcggcctggtgcagcctggcggaagcatgaaactgtcatgcgccgccagcggcttcatcttcagcgacgcctggatggactgggtgcggcaaagccccgagagaggcctggaatgggtcgccgagatcagaagcaaggccaacggcctggccccttactacgccgagagcgttaagggcagattcaccatcagccgggacgactctaaaagcagcgtgtacctgcaaatgaacaacctgagatccgaggacaccggcatctactactgcaccagcaccctgtactactttgattactggggccagggcacaacactgacagtgtcctccggttctacctccggcagcggcaagcccggcagcggcgagggctctcagatcgtgctgacacagtccccagccatcatgagcgcctttcctggagaaagagtgaccctgacctgcagcgcctcttctagcgtgcggtccagctatctggcttggtaccagcaaaagccaggctctagccctaagctgtggatctacagcacatctaatctggccagcggcgtgcctgctcggttcagcggcagcggcagcggaacaagctacagcctgaccatttcttccatggaagccgaggatgccgctagctacttctgccaccagtggtcctcttatcctcgtaccttcggcggaggcaccaagctcgagatcaag

SEQ ID NO: 159 - protein, 228 aa, synthetic construct
  REGION 1..228: note = Spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 160 - DNA, 684 bp, synthetic construct
  misc_feature 1..684: note = Spacer
  source 1..684: mol_type = other DNA
gagtccaaatacggtccgccatgcccaccatgcccagcacctcccgtggccggaccatcagtgttcctgttccccccaaaacccaaggacactctcatgatctcccggacccctgaggtcacctgcgtggtggtggacgtgagccaggaagatcccgaggtccagttcaactggtatgtggatggcgtggaagtgcataatgccaagacaaagccgcgggaggagcagttccagagcacgtaccgtgtggtcagcgtcctcaccgtcctgcaccaagactggctgaacggcaaggagtacaagtgcaaggtgtccaacaaaggcctcccgtcctccatcgagaaaaccatctccaaagccaaagggcagccccgagagccacaggtgtacaccctgcccccatcccaagaggagatgaccaagaaccaagtcagcctgacctgcctggtcaaaggcttctaccccagcgacatcgccgtggagtgggagagcaatgggcagccggagaacaactacaagaccacgcctcccgtgctggactccgacggctccttcttcctctactcccggctcaccgtggacaagagcaggtggcaggaggggaatgtgttctcatgctccgtgatgcatgaggctctgcacaaccactacacacagaagagcctctccctgtctctgggaaag

SEQ ID NO: 161 - protein, 11 aa, synthetic construct
  REGION 1..11: note = Spacer
SKYGPPCPPCP

SEQ ID NO: 162 - DNA, 33 bp, synthetic construct
  misc_feature 1..33: note = Spacer
  source 1..33: mol_type = other DNA
tccaaatacggtccgccatgcccaccatgccca

SEQ ID NO: 163 - DNA, 81 bp, synthetic construct
  misc_feature 1..81: note = Transmembrane domain
  source 1..81: mol_type = other DNA
ttttgggtgctggtggtggttggtggagtcctggcttgctattccttgctagtaacagtggcctttattattttctgggtg

SEQ ID NO: 164 - protein, 25 aa, synthetic construct
  REGION 1..25: note = Transmembrane domain
PILLTISILSFFSVALLVILACVLW

SEQ ID NO: 165 - DNA, 75 bp, synthetic construct
  misc_feature 1..75: note = Transmembrane domain
  source 1..75: mol_type = other DNA
cccatcctgctcaccatcagtatcctgtcctttttttccgtggctcttctcgtgattctggcttgcgtcctgtgg

SEQ ID NO: 166 - DNA, 126 bp, synthetic construct
  misc_feature 1..126: note = Costimulatory domain
  source 1..126: mol_type = other DNA
aaacgcggccgcaagaaactcctgtatatattcaaacaaccatttatgaggccagtacaaactactcaagaggaagatggctgtagctgccgatttccagaagaagaagaaggaggatgtgagctc

SEQ ID NO: 167 - DNA, 336 bp, synthetic construct
  misc_feature 1..336: note = Activation / Signaling domain
  source 1..336: mol_type = other DNA
agagtgaagttcagcaggagcgcagacgcccccgcgtaccagcagggccagaaccagctctataacgaactcaatctaggacgaagagaggagtacgatgttttggacaagcggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccaggggctcagtacagccaccaaggacacctacgacgcccttcacatgcaggccctgccccctcgc

SEQ ID NO: 168 - protein, 195 aa, synthetic construct
  REGION 1..195: note = IL7Ra Cytoplasmic domain
KKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 169 - DNA, 585 bp, synthetic construct
  misc_feature 1..585: note = IL7Ra Cytoplasmic domain
  source 1..585: mol_type = other DNA
aagaagcgcatcaagcccatcgtctggccaagcctgcccgaccacaagaagaccctcgagcacctgtgcaagaaaccgcgaaagaacctgaacgtgtcgttcaacccggagagcttcctggactgtcaaattcacagagttgatgacatccaggcacgcgacgaggtggagggcttccttcaggatacgttccctcagcagctggaggagagcgagaagcagcggctcgggggtgatgtgcagagccccaactgcccatccgaggacgtggtcatcactccggaatctttcggacgggacagctctctgacctgtctggccggcaacgtgtccgcgtgcgacgctcccatactgagctcctcccgctcgctcgactgccgggaaagtgggaagaatggccctcatgtatatcaggacctgctgttgtcgctagggacgaccaactccaccctgcctcccccattttcactgcaatccggcatcttgacactcaacccggtggcgcagggacagccgattcttacatcgctgggctccaaccaggaggaggcatacgtgaccatgtctagtttctaccagaaccaa

SEQ ID NO: 170 - protein, 22 aa, synthetic construct
  REGION 1..22: note = P2A peptide sequence
GSGATNFSLLKQAGDVEENPGP

SEQ ID NO: 171 - DNA, 66 bp, synthetic construct
  misc_feature 1..66: note = P2A peptide sequence
  source 1..66: mol_type = other DNA
ggatccggcgccacaaacttcagcctgctgaaacaggccggcgacgtggaggaaaacccaggccca

SEQ ID NO: 172 - protein, 21 aa, synthetic construct
  REGION 1..21: note = T2A peptide sequence
GSGEGRGSLLTCGDVEENPGP

SEQ ID NO: 173 - protein, 23 aa, synthetic construct
  REGION 1..23: note = E2A peptide sequence
GSGQCTNYALLKLAGDVESNPGP

SEQ ID NO: 174 - protein, 161 aa, synthetic construct
  REGION 1..161: note = Murine Thy1.1 staining marker
NPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 175 - DNA, 483 bp, synthetic construct
  misc_feature 1..483: note = Murine Thy1.1 staining marker
  source 1..483: mol_type = other DNA
aacccagccatcagcgtcgctctcctgctctcagtcttgcaagtgtcccgagggcagaaagtgaccagcctgacagcctgcctggtcaaccagaacctgagactggactgccggcacgagaacaacaccaaggacaacagcatccagcacgagttcagcctgaccagagaaaagcggaaacacgtgctgagcggcaccctgggaatccccgagcacacctatagaagcagagtgaccctgagcaaccagccttacatcaaagtgctgaccctggccaacttcaccaccaaggatgagggcgactacttctgcgagctgagagtgtctggcgccaatcctatgagcagcaacaagagcatcagcgtgtaccgggacaagctggtcaagtgtggcggcatctctctgctggtgcagaacacctcttggatgctgctgctcctgctgagcctgagtctgctgcaagccctggatttcatcagcctg

SEQ ID NO: 176 - protein, 239 aa, synthetic construct
  REGION 1..239: note = GFP Reporter
MVSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTITFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 177 - DNA, 717 bp, synthetic construct
  misc_feature 1..717: note = GFP Reporter
  source 1..717: mol_type = other DNA
atggtgtccaagggcgaagaactgttcaccggcgtggtgcccattctggtggaactggacggggatgtgaacggccacaagttcagcgttagaggcgaaggcgaaggggatgccacaaacggcaagctgaccctgaagttcatctgcaccaccggaaagctgcccgtgccttggcctacactggtcaccacactgacatacggcgtgcagtgcttcagcagataccccgaccatatgaagcagcacgacttcttcaagagcgccatgcctgagggctacgtgcaagagagaaccatcaccttcaaggacgacggcacctacaagaccagagccgaagtgaagttcgagggcgacaccctggtcaaccggatcgagctgaagggcatcgacttcaaagaggacggcaacatcctgggccacaaacttgagtacaacttcaacagccacaacgtgtatatcaccgccgacaagcagaagaacggcatcaaggccaacttcaagatccggcacaacgtggaagatggcagcgtgcagctggccgatcactaccagcagaacacacccatcggagatggccctgtgctgctgcccgataaccactacctgagcacccagagcaagctgagcaaggaccccaacgagaagcgggaccacatggtgctgctggaatttgtgacagccgccggaatcacccacggcatggatgagctgtacaag

SEQ ID NO: 178 - protein, 700 aa, synthetic construct
  REGION 1..700: note = Anti-PEG CAR 1
MARSPAQLLGLLLLWLSGARCEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAGSTSGSGKPGSGEGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 179 - protein, 700 aa, synthetic construct
  REGION 1..700: note = Anti-PEG CAR 2
MARSPAQLLGLLLLWLSGARCDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKGSTSGSGKPGSGEGSEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 180 - protein, 693 aa, synthetic construct
  REGION 1..693: note = Anti-PEG CAR 3
MARSPAQLLGLLLLWLSGARCQIVLTQSPAIMSAFPGERVTLTCSASSSVRSSYLCWYQQKPGSSPKLWIYSTSNLASGVPARFSGSGSGTSYSLTISSMEAEDAASYFCHQWSSYPRTFGGGTKLEIKGSTSGSGKPGSGEGSEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 181 - protein, 693 aa, synthetic construct
  REGION 1..693: note = Anti-PEG CAR 4
MARSPAQLLGLLLLWLSGARCQIVLTQSPAIMSAFPGERVTLTCSASSSVRSSYLAWYQQKPGSSPKLWIYSTSNLASGVPARFSGSGSGTSYSLTISSMEAEDAASYFCHQWSSYPRTFGGGTKLEIKGSTSGSGKPGSGEGSEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 182 - protein, 693 aa, synthetic construct
  REGION 1..693: note = Anti-PEG CAR 5
MARSPAQLLGLLLLWLSGARCEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSGSTSGSGKPGSGEGSQIVLTQSPAIMSAFPGERVTLTCSASSSVRSSYLCWYQQKPGSSPKLWIYSTSNLASGVPARFSGSGSGTSYSLTISSMEAEDAASYFCHQWSSYPRTFGGGTKLEIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 183 - protein, 693 aa, synthetic construct
  REGION 1..693: note = Anti-PEG CAR 6
MARSPAQLLGLLLLWLSGARCEVKLEESGGGLVQPGGSMKLSCAASGFIFSDAWMDWVRQSPERGLEWVAEIRSKANGLAPYYAESVKGRFTISRDDSKSSVYLQMNNLRSEDTGIYYCTSTLYYFDYWGQGTTLTVSSGSTSGSGKPGSGEGSQIVLTQSPAIMSAFPGERVTLTCSASSSVRSSYLAWYQQKPGSSPKLWIYSTSNLASGVPARFSGSGSGTSYSLTISSMEAEDAASYFCHQWSSYPRTFGGGTKLEIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 184 - protein, 483 aa, synthetic construct
  REGION 1..483: note = Anti-PEG CAR 7
MARSPAQLLGLLLLWLSGARCEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAGSTSGSGKPGSGEGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKSKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGP

SEQ ID NO: 185 - protein, 517 aa, synthetic construct
  REGION 1..517: note = Anti-PEG CCR 1
MPLLLLLPLLWAGALAEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAGSTSGSGKPGSGEGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKSKYGPPCPPCPPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGATNFSLLKQAGDVEENPGP

SEQ ID NO: 186 - protein, 734 aa, synthetic construct
  REGION 1..734: note = Anti-PEG CCR 2
MPLLLLLPLLWAGALAEVKLVESGGGLVQPGGSLKLSCATSGFTFSDYYIYWVRQTPEKRLEWVASISNGGGSTYYPDTLKGRFTISRDSAKNTLYLQMSRLKSEDTAMYYCARQHDSSYLAWFAYWGQGTLVTVSAGSTSGSGKPGSGEGSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPPTFGAGTKLELKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQGSGATNFSLLKQAGDVEENPGP